SEQUENCE LISTING (9 sequences)

SEQ ID NO: 1 - protein, 337 aa, Yarrowia lipolytica
MRSARRYLLVLLPVVALLYVLSTYFRGTPVEKPVARNMAGNHIVKYNMNKLQASSQAASHSEKVLILTPLARFYDEYWENLVALSYPHDLIELGFIVPRSKEGQQALEKLDRAVAKTQKGKDKFSKVTILRQEAEDALASHSEKDRHAMSVQKARRSAMSLARNSLFLSTIGPDTAWILWLDSDVVETPHTLIQDMTKHDKDVLVANCYQRYTNDQGKPDVRPYDYNSWVESQQGIKMAESMSPDDIILEGYGEMATYRLLMAKIYNADGDIHEEIALDGVGGTALLVKSEVHRDGAMFPPFPFYNLIETEGFAKMAKRLGYKPYGLPNYLVYHYNE

SEQ ID NO: 2 - DNA, 1014 bp, Yarrowia lipolytica
  source 1..1014: mol_type = genomic DNA
atgcgttcggctcgacggtatctgcttgtgcttctaccggtagtcgctctgctgtatgtgctgagcacctacttccgcggcacgcccgttgaaaaacccgtggctcgaaacatggccggaaatcacattgtcaagtacaatatgaacaagctgcaggcgtcgtcgcaggcggctagccacagcgagaaggtgctgattctcactcctctggctcgtttctacgacgagtactgggagaaccttgtggctctgagctaccctcacgacctcatcgagctgggcttcattgttcctcgaagcaaggagggccaacaggcgctggagaagctcgatcgagctgtcgcgaagacccagaagggcaaggacaagttttctaaggtgaccattctgcgacaggaggctgaggacgctttggcttcacattcggaaaaggaccgacacgcgatgtctgtgcagaaggcccggcgaagtgccatgtctctggctcgaaactcactgttcctgtccaccattggccccgacactgcatggattctgtggctggactcggacgtggtggagaccccccataccctcatccaggacatgaccaaacatgacaaggacgttctggtggctaattgctatcagcgatacaccaacgaccagggcaagcccgacgtgcggccctacgactacaactcgtgggtcgagtcgcaacagggcatcaagatggccgagtccatgtcccctgacgacatcattctcgagggctacggagaaatggccacctaccgactgctcatggccaagatctacaacgccgatggagacattcacgaggaaattgccctcgacggtgtcggaggaaccgctctgctggttaagtccgaggtgcaccgtgatggagccatgtttcctcctttccccttttacaacctcatcgagacagagggttttgccaagatggccaagcgacttggctacaagccttacggtcttcccaactatctcgtctaccattacaatgagtag

SEQ ID NO: 3 - protein, 1222 aa, Yarrowia lipolytica
MDSYNKFGFNSETGEPNTGPQDTSGSVPSEHRGDGSFSPDIGRKKSLIRPDRARLDPDSRTYHYSQLAAQQQSRINVLPSSTGNDPILEEHYMRETGEIQPGQGYDQPYDNAVNQEVLEEENEESYREGEEDPYAYSGRQVYGLNDEVQTEAYVRKSDEISSSEEQAADTQPPIIDPPVNEKVQLSRGGGTLKRHNSVKRRMGTQPKQISLWMTYCQIVTFWAPAPVLKMFGMPQKARQDAWREKIGLITVILYIAAFVAYLTFGFTITVCSSSITRVRNGEVNSGNLIINGKQYDFTSMNHPPTVEVPTGGNFMYPPVNAGSMDGSLLFQNVNNHCKGLITPKPDCKIPYNGDSVAWYMPCQLLNQDGSSQPNTTEQFYPGYACHTSTTARNVYYHQLVDAGEVYFTWDDIKNSTRNLAVFNGAVIDLNMVNFILKDDLNYPEIFDTLRDDDSIKGTDISRNMANGPDRQAARCLQEIAKVGYIDTEQIGCLASQVVLYVSLVFIISIVAVKFLFAIYYKWAISAKQGAFPIDNKQMNQRENEIEDWSENIYSQAPIKRVDRDQPKSSATPFRGLIKHRFNQHDNKNKRMSTMVTQVGESGTAPSVYSRNSAAIDSNTDVGGVVYDYDSGSQPYEAPTVNVVPQPPIDYQPFNYPLAHTMCLVTAYSESIDGLRTTLDSITTTDYPNSHKLIVIIADGIIKGSGNDMSTPDICLSMMCDMPIPEDQVRPFSYVSTSSGTKRHNMAKVYSGFYAYDDATVPPEKQQRVPVVTIVKCGTPAEANEAKPGNRGKRDSQIILMSFLQRVMFDERMTELEHEMFNGIWRVTGILPDFYEIILMVDADTKVFPDSLTHMVAEMVKDPEIMGLCGETKIANKRDSWVTAIQVFEYYISHHQAKAFESVFGGVTCLPGCFSMYRIKAPKGGDGYWVPILANPDIVERYADNVIDTLHKKNLLLLGEDRYLTSLMLKTFPTRKQVFVSKAVCKTIVPDKFSVLLSQRRRWINSTVHNLMELVLVKDLCGVFCISMQFVVFIELVGTLILPAAISFTIYVVIIAIVRKPTPVMSLVLLALILGLPGLLIIITASRWSYVVWMFIYLLSLPIWNFVLPVYAYWKFDDFSWGDTRQVAGEKKGSGGHDEEDGEFDSSQIVMKRWRDFEMDKRRYVPLPPQNAWMSDPHMPSYNHQGFSANSSPALRPQGSSHVYSDSVAGASDYNVAQGHEVL

SEQ ID NO: 4 - DNA, 3669 bp, Yarrowia lipolytica
  source 1..3669: mol_type = genomic DNA
atggattcgtacaacaaattcggattcaactcggagacgggtgagcccaacactggcccccaggacacatctggcagtgtccccagtgaacacagaggcgacggctcgttctcccccgacattggacgaaagaagtctctcattcgacctgaccgagctcgactcgaccccgactcgcgaacttatcactactcgcagctggccgcccagcagcaaagcagaatcaacgtgcttcccagttccaccggaaatgaccctattctcgaggagcactacatgcgcgagactggcgagatccagcctggtcagggatacgatcagccctacgacaacgccgtgaaccaggaggtgctagaggaggaaaacgaggaatcatacagagagggagaggaggacccttacgcctactcgggccgtcaggtctatggtctcaatgacgaggtccaaaccgaggcctacgtgcgaaagtcggacgaaatctcctcatcagaagaacaggctgccgacacccaaccgcccatcatcgacccacctgtcaacgaaaaggttcagctcagccgtggaggaggaacactgaagcgacacaactccgtcaagcgacgaatgggcacccaacccaagcagatttcgctgtggatgacctactgtcagattgttaccttctgggcccccgctcctgtgctcaagatgtttggcatgccccaaaaagcccgacaggacgcctggcgagaaaagattggcctcatcactgttattctttacattgccgcctttgttgcatacctgactttcggcttcaccatcacagtttgttccagttccatcacccgagtgcgaaacggagaagtcaactctggaaacctcatcattaatggtaagcaatacgactttacctctatgaaccaccctcccacagtcgaggttcctactggcggtaacttcatgtaccctcctgttaatgctggatccatggacggttctttgctgttccagaacgtcaacaaccactgcaagggtctaatcacccccaagcccgactgtaaaattccctacaacggcgacagcgttgcctggtatatgccctgccagctgctcaaccaggacggatcttcccaacccaacactactgagcagttctaccccggctacgcatgccacacttccaccaccgctcgaaacgtctactaccaccagctggttgatgccggtgaggtctacttcacctgggacgatatcaagaactctactcgaaacctggctgtgttcaacggagctgtcattgacctgaacatggtcaacttcattcttaaggacgacctcaactaccccgagatctttgacactctgcgagatgacgattccatcaagggtactgatatctctcgtaatatggccaacggtcctgatcgacaggctgcccgatgtctgcaagagattgccaaggttggatacattgataccgagcagattggatgtctggcttcccaggttgttctgtatgtatctctggttttcattatctccattgtcgccgtcaagttcctatttgctatctactacaagtgggccatttctgccaagcagggagctttccccattgacaacaagcagatgaaccagcgagagaacgagattgaggactggtccgagaacatttacagccaggctcccatcaagcgagtggaccgagaccagcctaagtccagtgccactcccttccgtggcctcatcaagcaccgattcaaccagcacgacaataagaacaagcgaatgagcacaatggtgacccaggttggtgagagtggaactgctccttccgtctactctcgaaactccgctgcgatcgactccaacactgatgttggcggtgttgtctacgattacgactctggatctcagccctacgaggcgcccaccgtcaacgttgtgccccagccccccatcgactaccaaccgttcaactaccctctcgcccacaccatgtgtctggtgactgcatactccgagtcgattgacggtctgagaaccacccttgactctatcaccacaaccgactaccctaactcccataaactcattgttatcattgccgatggtattattaagggttccggtaacgacatgtctacccctgacatttgtctaagtatgatgtgtgatatgcctatccccgaagaccaggtgagacccttctcttacgtatctacttcttctggtacaaagcgtcataacatggccaaggtctactctggtttctacgcctacgatgatgccacagttccacctgagaagcagcagcgagtccccgttgtgactattgtcaagtgcggtacgcctgctgaggccaacgaggccaagcctggtaaccgaggtaagcgagactcccagatcattctcatgtcgttcttgcagcgtgtcatgttcgacgagcgaatgaccgagctggagcacgagatgttcaacggtatctggcgagtgactggtattcttcccgacttttatgaaatcattctcatggtggatgccgatactaaggtgttccctgactctctgacccacatggtggctgagatggtcaaggatcccgagattatgggactatgtggagaaaccaagattgccaacaagcgagactcttgggtcactgcgatccaggtgtttgaatactacatttctcatcaccaggctaaggcctttgagtccgtctttggtggtgtcacttgtctgcctggttgtttctccatgtaccgaatcaaggctcccaagggcggagacggctactgggtgcccattcttgccaaccctgatatcgtcgagcgatacgctgacaacgtgattgacactctgcataagaagaacctgttgcttctcggagaagatcgatacctgacctccctcatgctcaagaccttccccacgcgaaaacaggtgtttgtgtccaaggctgtctgcaagaccattgttcccgacaagttttctgttttgctgtctcagcgaagacgatggattaactccactgtgcataacctgatggagctggtgctggtcaaggatctatgtggtgtcttctgtatttccatgcagtttgttgtcttcatcgagcttgttggtaccctgattcttcccgccgccatctcgttcaccatctacgttgtcattattgccattgtgagaaaacctacgcctgtcatgtcgctggttctgctggctctgattttgggtctgccgggtcttctcattatcatcacagcatctcgatggtcatacgtcgtgtggatgttcatctacctgctatctcttcccatttggaactttgtgctccccgtctacgcatactggaagtttgatgatttctcctggggtgacactcgacaggttgctggtgaaaagaagggctctggaggccacgacgaggaggatggtgaatttgactcgtctcagattgtcatgaagcgatggagagactttgaaatggacaagcgacgctacgtgccactgcctccccagaacgcgtggatgagcgatcctcacatgccttcttacaaccaccaaggattttctgctaactcttcccctgctcttagaccccagggctcttctcatgtctactcggactcggttgccggagccagtgactacaacgttgctcaaggccacgaagtactttaa

SEQ ID NO: 5 - protein, 552 aa, Yarrowia lipolytica
MKFSAVSIAAALASLVAAADVPSIDIVGNKFFYSNNGSQFYIRGVAYQQDSANETAKETFSDPLSDGDACKRDLPYLVQLKTNVLRVYAIDTTKDHDTCMQLFNDAGIYVISDLSQPGESINRNSPSWDVDLYQRYTSVVDTMQKYPNMLGFFAGNEVTNNNTNTDASPYVKAAIRDMKSYMKEKSYRSIPVGYATNDDEKTRDDLATYFQCGNASEAADFYGINIYEWCGDATFESSGYQQRTEEYKNFTIPIFFSEYGCNRVKPRQFSDVPVLFGPNMTDVWSGGIVYMYFEEQNQYGLVSVSGSSVSTLDDFNNLSSQMAKISPSSANTNDYKPSQTAGGVSCPDFNANWKGSPTLPPTPKVGTCQCINDGSSCVVNSDTNAKNYGDLFGELCGNLDVDCSEIQSNGTSGKYGDLSFCDAKQKLNFLMGKYYDSQNGNSQACDFSSQASIVQSPKTGSNCQNAQATAVSSGSSGSSGSSGSGSSGSGSSGSSGSGSGSAKGSAAAAASGSAAASSSKGGNKKSAGASLAAEKVVASVVFGAIAIMAVIV

SEQ ID NO: 6 - DNA, 1659 bp, Yarrowia lipolytica
  source 1..1659: mol_type = genomic DNA
atgaagttctcagcggtctcaatcgctgctgccctggcctcgctggtggcagcagcagacgtgccctctattgacattgtgggcaacaagttcttctactccaacaacggctctcaattctacatccgaggagtggcctaccagcaggactcggccaacgagacagccaaggagaccttttcggaccctctgtcggacggcgacgcctgtaagcgagacctgccctacctggtgcagctcaagaccaacgtgcttcgagtgtacgccatcgacaccaccaaggaccacgacacatgcatgcagctcttcaacgacgccggcatctacgtcatttccgatctctcacagcccggcgagtccatcaaccgaaactcgccctcgtgggacgtggatctgtaccagcgatacaccagcgtagtggacaccatgcaaaagtaccccaacatgctgggtttctttgccggaaacgaggtcactaacaacaacaccaacaccgatgcgtctccctacgtcaaggctgccatccgagacatgaagtcgtacatgaaggagaagtcgtaccgatccattcccgtcggatacgccaccaacgatgacgagaagacccgagacgacctggccacctatttccagtgcggcaacgcctccgaggcagccgatttctacggaatcaacatctacgagtggtgtggagacgccacctttgagtcctccggttaccagcagcgaaccgaggagtacaagaacttcaccatccccatcttcttctccgagtacggctgcaaccgagtcaagccgcgacagttctccgacgtgcccgttctgtttggccccaacatgaccgacgtctggtccggaggaatcgtctacatgtactttgaggagcagaaccagtacggtctggtgtctgtttccggaagctccgtctccactctcgacgacttcaacaacctgtcttcgcagatggctaagatttctccttcctccgccaacaccaacgactacaagccctctcagaccgctggtggcgtctcgtgccccgacttcaacgccaactggaaaggctctcccaccctgcctcccacccccaaggtcggaacctgtcagtgcatcaacgatggctcttcttgtgtcgtcaactcggacactaacgccaagaactacggcgatctctttggagagctgtgcggtaacctcgatgtggactgctccgagatccagtccaacggaacctccggcaagtacggagacctgtccttctgtgacgccaagcagaagctcaacttcctcatgggcaagtactacgactcccagaacggcaactctcaggcctgcgacttttcttcccaggcctccattgtccagagccccaagaccggatccaactgccagaacgcccaggccactgctgtctcttccggctcgtccggctcttccggctcgtccggctctggatcttcgggctctggatcgtccggatcctcgggctctggctcgggctctgctaagggctctgctgctgccgctgcctctggctcggccgccgcctcgtcctccaagggcggaaacaagaagagcgccggcgcctcgctggctgccgagaaggtggttgcctccgtcgtgtttggcgccatcgccatcatggccgttattgtttaa

SEQ ID NO: 7 - DNA, 12810 bp, synthetic construct
  source 1..12810: mol_type = other DNA
ggttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctgcaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaacacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctggcctaggaagcgacttccaatcgctttgcatatccagtaccacacccacaggcgtttgtgctactctactgatagcaatagatgcgtcataattggttggcccgctgagcctccacaggatactattgcacataccctggtcatgtgcagatcagctcatttgtggagactctggagtaacttagacgacgcctggttcaattgccgcaatgtgcgcccacgcagataatgtattgaggggtggagcgcctcttggggacttgctgtacttgtacgggatattaaacgcactcagcaagaccatgacgtaaaacacacctactgtacgatacgtactgtaggtattgtactcgtacccggtactacaaatagtacgatactatacggagtgtatttgtaccttgatatacgactggcggagtgaagagaaggagttgaacaagaccagatggggatatcagccccagtgctttgtattacaagtacgagtacttaatagatactgtaaggctattgatacggatggcagtaagtcattgagtaagcaattgtggcccagcatctcccctacgtacttgtaccataccccatggagacaccaatggtctttcacgcacactgtcgtgtgctgtatcgcagaatcgggtgtccaaccaaatgccgttacccccacgtcacagccgatagacagatacaccatcaataccagcaggttgtatcatgcggttggctgaaggtaagctgattggtctaaaaactgtagctgtcctaattcaacgagcgctatttggggccaaccacctcggccaagcggcctttaatctgcgtgccccagaggcgtctaatgaggctctggccgccactgtaggagtgtttctctgtgcgcacacgcagttttgagtttgggcgactttccctttttcccaattgcgtacacacacagctccgagctaagcgctgtccttgaaccttctccctcttttccctctttttctcttccccttcccctcctccacattaaggccaaatcctgaattgcaccaactagtacaacgacaacaatggacaagaagtactccatcggtttggacattggtactaactctgtcggctgggccgtcatcaccgacgagtacaaggttccctccaagaagttcaaggtccttggcaacaccgaccgacactctatcaagaagaacctgatcggtgctctgctgttcgactctggcgagactgccgaggccacccgactgaagcgaaccgctcgacgccgatacacccgacgaaagaaccgaatctgttacctccaggagatcttcagcaacgagatggctaaggtcgacgactccttcttccaccgactcgaggagtctttcctggtcgaagaggataagaagcacgagcgacaccccatcttcggcaacattgttgatgaggttgcctaccatgagaagtaccccaccatctaccacctccgaaagaagctcgtcgactccactgacaaggctgacctccgactcatctaccttgctctcgcccacatgatcaagttccgaggtcacttcctcattgagggtgatctcaaccccgacaactccgacgttgacaagctgttcatccagctcgtccagacctacaaccagctctttgaggagaaccctatcaacgcttctggtgttgacgccaaggccattctctccgcccgactctctaagtcccgacgactcgagaacctcattgcccagctgcccggcgagaagaagaacggcctcttcggtaacctgattgctctctctcttggtctgacccccaacttcaagtccaactttgacctcgccgaggacgccaagctccagctgtccaaggacacctacgatgacgatctggacaacctcctggcccagatcggtgaccagtacgccgatctcttccttgccgccaagaacctctccgacgccatcctgctctccgacatcctccgagtcaacaccgagattaccaaggctcctctgtctgcctctatgatcaagcgatacgacgagcaccaccaggatctcactcttctcaaggctctcgtccgacagcagctccccgagaagtacaaggagattttctttgaccagtccaagaacggttacgctggctacattgacggtggtgcttcccaggaagagttttacaagttcatcaagcctattctggagaagatggacggtaccgaggagctgctcgtcaagctcaaccgagaggacctccttcgaaagcagcgaaccttcgataacggctccatcccccaccagatccacctgggtgagctccacgccattctccgaagacaagaggacttctaccccttcctaaaggataaccgagagaagatcgagaagattctcaccttccgaatcccctactacgtcggtcccctcgctcgaggtaactcccgatttgcttggatgacccgaaagtccgaggagactatcaccccctggaactttgaagaggtagtcgacaagggtgcctccgcccagtctttcattgagcggatgaccaacttcgataagaacctccccaacgagaaggtccttcccaagcactctctcctctacgagtacttcaccgtctacaacgagctgaccaaggtcaagtacgttaccgagggcatgcgaaagcccgctttcctctctggtgagcagaagaaggccattgtcgacctcctgttcaagactaaccgaaaagtcaccgtcaagcagctcaaggaagactacttcaagaagattgagtgcttcgactccgtcgagatttccggtgtcgaggaccgattcaacgcctccctcggcacctaccacgatcttctgaagatcatcaaggacaaggactttcttgataacgaggagaacgaggacattctcgaggacatcgtcctcaccctcacccttttcgaggatcgagagatgatcgaggagcgactcaagacctacgcccatctcttcgacgacaaggtcatgaagcaactcaagcgacgacgatacactggctggggccgactttcccgaaagctcatcaacggcatccgagacaagcagtctggcaagaccatcctggacttcctgaagtccgacggtttcgccaaccgaaacttcatgcagctcatccacgacgactctcttaccttcaaagaggatatccagaaggcccaggtttctggccagggcgactccctccacgagcacattgccaacctcgccggatcccccgccatcaaaaagggtatcctccagaccgtcaaggttgtcgacgaactcgtgaaggtcatgggccgacacaagcccgagaacatcgttatcgagatggcccgagagaaccagaccacccagaagggtcagaagaactcccgagagcgaatgaagcgaatcgaagagggtatcaaggagctcggttcccagattctcaaggagcaccccgtcgagaacacccagctccagaacgagaaactctacctgtactacctccagaatggccgagacatgtacgttgaccaggagctcgacatcaaccgactctccgactacgacgtcgaccacattgttcctcagtccttcctcaaggacgactccatcgacaacaaggttctgacccgatctgacaagaaccgaggtaagtccgacaacgttccctccgaagaggtcgttaagaagatgaagaactactggcgacagcttctcaacgccaaactgatcacccagcgaaagtttgacaacctcaccaaggccgagcgaggtggtctgtccgagctggacaaggccggcttcattaagcgacagctggtcgagactcgacagatcaccaagcacgtcgcccagatcctcgactcccgaatgaacaccaagtacgacgagaacgacaagctcatccgggaggtcaaggtcatcaccctgaagtctaagcttgtctccgacttccgaaaggacttccagttctacaaggtccgagagatcaacaactaccaccacgcccacgacgcctacctcaacgccgttgttggtaccgccctcatcaagaagtatcccaagctcgagtccgagttcgtttacggcgactacaaggtttacgatgtccgaaagatgattgccaagtccgagcaggagatcggtaaggccaccgccaagtactttttctactccaacatcatgaatttcttcaagaccgagatcactctcgccaacggtgagattcgaaagcgacccctgattgagactaatggtgagactggtgagatcgtctgggataagggccgagacttcgccaccgtccgaaaggtcctgtccatgccccaggtcaacattgtcaagaagaccgaggtccagaccggtggcttctccaaggagtccattctccccaagcgaaactccgacaaactcatcgcccgtaagaaggactgggatccgaagaagtacggtggtttcgattctcccaccgttgcctactccgtcctcgttgttgctaaagtcgagaagggtaagtctaagaaactcaagtccgtgaaggagctactcggtatcaccatcatggagcgatcttcttttgagaagaaccccattgacttcctcgaggccaagggttacaaagaggtcaagaaggacctgattatcaagctgcccaagtactccctctttgagctcgagaacggccgaaagcgaatgctggcttccgctggtgagctgcagaagggcaacgagctcgctctgccctccaagtacgtcaacttcctctacctggcctcccactacgagaagctcaagggctcccccgaggacaacgagcagaagcagctgttcgttgagcagcacaagcactacctcgacgagatcatcgagcagatctccgagttctccaagcgagtcatcctcgctgacgccaaccttgataaggttctctctgcttacaacaagcaccgggacaagcccatccgagagcaggccgagaatatcatccacctcttcactctcaccaacctcggcgctcctgctgccttcaagtacttcgacaccaccattgaccgaaagaggtacacctccaccaaggaagtcctcgacgccaccctgatccaccagtccatcaccggcctctacgaaacccgaatcgacctctcccagctcggcggtgactctcgagccgaccccaagaagaagcgaaaagtctaaatatccgaagatcaagagcgaagcaagttgtaagtccaggacatgtttcccgcccacgcgagtgatttataacacctctcttttttgacacccgctcgccttgaaattcatgtcacataaattatagtcaacgacgtttgaataacttgtcttgtagttcgatgatgatcatatgattacattaatagtaattactgtatttgatatatatactaattacaatagtacatattagaacatacaatagttagtgccgtgaagtggcttaaaataccgcgagtcgattacgtaatattattacctcttgcccatcgaacgtacaagtactcctctgttctctccttcctttgctttgtgcacgaagaactgcggtcaggtgacacaactttttccatctcagggtgtgtcgcgtgtgcttcatccaaactttagttggggttcgggttcgcgcgagatgatcacgtgccctgatttggtgtcgtcccccgtcgcgctgcgcacgtgatttatttatttccggtggctgctgtctacgcggggccttctctgcccttctgtttcaaccttcgggcggttctcgtaaccagcagtagcaatccatttcgaaactcaaagagctaaaaacgttaaacctcagcagtcgctcgacgaatgggctgcggttgggaagcccacgaggcctatagccagagcctcgagttgacaggagcccagacgccttttccaacggcaacttttatataaaatggcaatgtattcatgcaattgcggccgtgtcaggttggagacactggaccacactctccattgcttcctgaggagatggatcattgctagtgcatctacgcgcagcaatcccgcaagctcgacaaccgtagatgggctttggtgggccaatcaattacgcaacccgcacgttaaattgtatgaggaaggaaggccacggtacaaagtgggtggtcttcacccagtggttgttggtggcgtcatgcagaccatgcattggggatagcacagggttggggtgtcttgtggactcaatgggtgaaaggagatggaaaagggcggtgaaaagtggtagaatcgaaatccctgacgtcaatttataaagtaaaatgcgtttctgccattttgctcccctccttctttcgcaatcgcctccccaaaagttgtcgtggcagtacacatgcttgcatacaatgaagctaatccggcttgctcagtagttgctatatccaggcatggtgtgaaacccctcaaagtatatataggagcggtgagccccagtctggggtcttttctctccatctcaaaactactttctcacaatgaagaagctgatgagtccgtgaggacgaaacgagtaagctcgtccttctttcgtccaatgtcgggttttagagctagaaatagcaagttaaaataaggctagtccgttatcaacttgaaaaagtggcaccgagtcggtgcttttggccggcatggtcccagcctcctcgctggcgccggctgggcaacatgcttcggcatggcgaatgggactaaacttcgagctaatccagtagcttacgttacccaggggcaggtcaactggctagccacgagtctgtcccaggtcgcaatttagtgtaataaacaatatatatattgagtctaaagggaattgtagctattgtgattgtgtgattttcgtcttgctggttcttattgtgtcccattcgtttcatcctgatgaggacccctggaaccggtgttttcttagtctctgcaatcgctagtcttgttgctatgacagttgcgtcgacactattcaggtcatctatcggttattctgatattataatacctccggatcgatgtacctgatttatacttgcagcaatgtttacttcttatcgcgatacacgaatgtgatacggatcaaagtaagcaggactacgataagataacgaatgcggtgcagtccatgtcgattaggtatagatacatttattttgtgttatgttacattttggggggatactgtcctacttgtagtacctacttgtagtggcgcgtctattcctttgccctcggacgagtgctggggcgtcggtttccactatcggcgagtacttctacacagccatcggtccagacggccgcgcttctgcgggcgatttgtgtacgcccgacagtcccggctccggatcggacgattgcgtcgcatcgaccctgcgcccaagctgcatcatcgaaattgccgtcaaccaagctctgatagagttggtcaagaccaatgcggagcatatacgcccggagccgcggcgatcctgcaagctccggatgcctccgctcgaagtagcgcgtctgctgctccatacaagccaaccacggcctccagaagaagatgttggcgacctcgtattgggaatccccgaacatcgcctcgctccagtcaatgaccgctgttatgcggccattgtccgtcaggacattgttggagccgaaatccgcgtgcacgaggtgccggacttcggggcagtcctcggcccaaagcatcagctcatcgagagcctgcgcgacggacgcactgacggtgtcgtccatcacagtttgccagtgatacacatggggatcagcaatcgcgcatatgaaatcacgccatgtagtgtattgaccgattccttgcggtccgaatgggccgaacccgctcgtctggctaagatcggccgcagcgatcgcatccatggcctccgcgaccggctgcagaacagcgggcagttcggtttcaggcaggtcttgcaacgtgacaccctgtgcacggcgggagatgcaataggtcaggctctcgctgaattccccaatgtcaagcacttccggaatcgggagcgcggccgatgcaaagtgccgataaacataacgatctttgtagaaaccatcggcgcagctatttacccgcaggacatatccacgccctcctacatcgaagctgaaagcacgagattcttcgccctccgagagctgcatcaggtcggagacgctgtcgaacttttcgatcagaaacttctcgacagacgtcgcggtgagttcaggctttttcatatgggtacctgagaacatttttgtgtctaggtgtttgtgtttggactgcgatcagtgaagaaaagaagaggaaaaattgtgcaagaaattttgctttcaagacttggctgatgcagcagggtaactctgggacacagacctatgtttgtggttaaactcaatgcacgtggtacgtgcgtggagcgcttacccatccaagggtgtggacatggaaccgacggtccgtggagttgtgtaatgtcattttggcgactcttgaagcaaggctataaaaaaattgtgtggcttgagtcttatcgagctcggtcactacaagagttaatcttcctgtctcaggcagacaggtcaggcagggttacttttgggtgtgctgtaactcactgtatggccgttagtgcgcatagacgttgtacatactggaccgaattgtagcgtgctcaatagggccaataaagctattgtagggatccgaattttcagaacctaatttatctgttacccggcctgtggctcgcacagcttaaaaatggtcaaactttccccttcttgtctttttttcctcacattcatcaggttcttgtcttgatctttcaagtgagtattaattaccgaccttggttcttcattgggagagcattggaagccgtggtgcagcaaccacaaaacggttcttccccttcgataccttcttgcctgcctttcaatacaagtcggctcgattagcggtggtcgcccccgccagcggagaacatggaactaacccagaatgagagctaagtggagaaagaagagagtcagacgactcaagcgaaagcgccgcaaggtccgagctcgatccaaataagcggtttttaacggagatttaacactaaatcgaagaacttttcccgtttcatttgcgaatgagctcgttaacaaaatcccccagtttttttatccagctgtaaggattgacattagtaatgaattattgtttggtatatttaaatctgtagttcctttctgtccgtgtcggcaactgtcgtactcgtgatttacttgtattgacgaatacttactgtagcgcactctgctgctactggtcgtaaggatgtgctatttcggtgtatggtgggttttttgggggtcggaaccgaagactgttacacgggcacggctcgttgtgtacacgcacagagctcttgcgagtcatgttgtagctagctcgtcgtgttcaggaactgttcgatggttcggagagagtcgccgcccagaacatacgcgcaccgatgtcagcagacagccttattacaagtatattcaagcaagtatatccgtagggtgcgggtgatttggatctaaggttcgtactcaacactcacgagcagcttgcctatgttacatccttttatcagacataacataattggagtttacttacacacggggtgtacctgtatgagcaccacctacaattgtagcactggtacttgtacaaagaatttattcgtacgaatcacagggacggccgccctcaccgaaccagcgaatacctcagcggtcccctgcagtgactcaacaaagcgatatgaacatcttgcgatggtatcctgctgatagtttttactgtacaaacacctgtgtagctccttctagcatttttaagttattcacacctcaaggggagggataaattaaataaattccaaaagcgaagatcgagaaactaaattaaaattccaaaaacgaagttggaacacaaccccccgaaaaaaaacaacaaacaaaaaacccaacaaaataaacaaaaacaaaataaatatataactaccagtatctgactaaaagttcaaatactcgtacttacaacaaatagaaatgagccggccaaaattctgcagaaaaaaatttcaaacaagtactggtataattaaattaaaaaacacatcaaagtatcataacgttagttattttattttatttaataaaagaaaacaacaagatgggctcaaaactttcaacttatacgatacataccaaataacaatttagtatttatctaagtgcttttcgtagataatggaatacaaatggatatccagagtatacacatggatagtatacactgacacgacaattctgtatctctttatgttaactactgtgaggcattaaatagagcttgatatataaaatgttacatttcacagtctgaacttttgcagattacctaatttggtaagatattaattatgaactgaaagttgatggcatccctaaatttgatgaaagatgaaattgtaaatgaggtggtaaaagagctacagtcgttttgttttgagataccatcatctctaacgaaatatctattaaaaatctcagtgtgatcatgagtcattgccatcctggaaaatgtcatcatggctgatatttctaactgtttacttgagataaatatatatttacaagaacttcccttgaaattaatttagatataaaatgtttgcgggcaagttactacgaggaataaattatatctaga

SEQ ID NO: 8 - DNA, 12810 bp, synthetic construct
  source 1..12810: mol_type = other DNA
ggttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctgcaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaacacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctggcctaggaagcgacttccaatcgctttgcatatccagtaccacacccacaggcgtttgtgctactctactgatagcaatagatgcgtcataattggttggcccgctgagcctccacaggatactattgcacataccctggtcatgtgcagatcagctcatttgtggagactctggagtaacttagacgacgcctggttcaattgccgcaatgtgcgcccacgcagataatgtattgaggggtggagcgcctcttggggacttgctgtacttgtacgggatattaaacgcactcagcaagaccatgacgtaaaacacacctactgtacgatacgtactgtaggtattgtactcgtacccggtactacaaatagtacgatactatacggagtgtatttgtaccttgatatacgactggcggagtgaagagaaggagttgaacaagaccagatggggatatcagccccagtgctttgtattacaagtacgagtacttaatagatactgtaaggctattgatacggatggcagtaagtcattgagtaagcaattgtggcccagcatctcccctacgtacttgtaccataccccatggagacaccaatggtctttcacgcacactgtcgtgtgctgtatcgcagaatcgggtgtccaaccaaatgccgttacccccacgtcacagccgatagacagatacaccatcaataccagcaggttgtatcatgcggttggctgaaggtaagctgattggtctaaaaactgtagctgtcctaattcaacgagcgctatttggggccaaccacctcggccaagcggcctttaatctgcgtgccccagaggcgtctaatgaggctctggccgccactgtaggagtgtttctctgtgcgcacacgcagttttgagtttgggcgactttccctttttcccaattgcgtacacacacagctccgagctaagcgctgtccttgaaccttctccctcttttccctctttttctcttccccttcccctcctccacattaaggccaaatcctgaattgcaccaactagtacaacgacaacaatggacaagaagtactccatcggtttggacattggtactaactctgtcggctgggccgtcatcaccgacgagtacaaggttccctccaagaagttcaaggtccttggcaacaccgaccgacactctatcaagaagaacctgatcggtgctctgctgttcgactctggcgagactgccgaggccacccgactgaagcgaaccgctcgacgccgatacacccgacgaaagaaccgaatctgttacctccaggagatcttcagcaacgagatggctaaggtcgacgactccttcttccaccgactcgaggagtctttcctggtcgaagaggataagaagcacgagcgacaccccatcttcggcaacattgttgatgaggttgcctaccatgagaagtaccccaccatctaccacctccgaaagaagctcgtcgactccactgacaaggctgacctccgactcatctaccttgctctcgcccacatgatcaagttccgaggtcacttcctcattgagggtgatctcaaccccgacaactccgacgttgacaagctgttcatccagctcgtccagacctacaaccagctctttgaggagaaccctatcaacgcttctggtgttgacgccaaggccattctctccgcccgactctctaagtcccgacgactcgagaacctcattgcccagctgcccggcgagaagaagaacggcctcttcggtaacctgattgctctctctcttggtctgacccccaacttcaagtccaactttgacctcgccgaggacgccaagctccagctgtccaaggacacctacgatgacgatctggacaacctcctggcccagatcggtgaccagtacgccgatctcttccttgccgccaagaacctctccgacgccatcctgctctccgacatcctccgagtcaacaccgagattaccaaggctcctctgtctgcctctatgatcaagcgatacgacgagcaccaccaggatctcactcttctcaaggctctcgtccgacagcagctccccgagaagtacaaggagattttctttgaccagtccaagaacggttacgctggctacattgacggtggtgcttcccaggaagagttttacaagttcatcaagcctattctggagaagatggacggtaccgaggagctgctcgtcaagctcaaccgagaggacctccttcgaaagcagcgaaccttcgataacggctccatcccccaccagatccacctgggtgagctccacgccattctccgaagacaagaggacttctaccccttcctaaaggataaccgagagaagatcgagaagattctcaccttccgaatcccctactacgtcggtcccctcgctcgaggtaactcccgatttgcttggatgacccgaaagtccgaggagactatcaccccctggaactttgaagaggtagtcgacaagggtgcctccgcccagtctttcattgagcggatgaccaacttcgataagaacctccccaacgagaaggtccttcccaagcactctctcctctacgagtacttcaccgtctacaacgagctgaccaaggtcaagtacgttaccgagggcatgcgaaagcccgctttcctctctggtgagcagaagaaggccattgtcgacctcctgttcaagactaaccgaaaagtcaccgtcaagcagctcaaggaagactacttcaagaagattgagtgcttcgactccgtcgagatttccggtgtcgaggaccgattcaacgcctccctcggcacctaccacgatcttctgaagatcatcaaggacaaggactttcttgataacgaggagaacgaggacattctcgaggacatcgtcctcaccctcacccttttcgaggatcgagagatgatcgaggagcgactcaagacctacgcccatctcttcgacgacaaggtcatgaagcaactcaagcgacgacgatacactggctggggccgactttcccgaaagctcatcaacggcatccgagacaagcagtctggcaagaccatcctggacttcctgaagtccgacggtttcgccaaccgaaacttcatgcagctcatccacgacgactctcttaccttcaaagaggatatccagaaggcccaggtttctggccagggcgactccctccacgagcacattgccaacctcgccggatcccccgccatcaaaaagggtatcctccagaccgtcaaggttgtcgacgaactcgtgaaggtcatgggccgacacaagcccgagaacatcgttatcgagatggcccgagagaaccagaccacccagaagggtcagaagaactcccgagagcgaatgaagcgaatcgaagagggtatcaaggagctcggttcccagattctcaaggagcaccccgtcgagaacacccagctccagaacgagaaactctacctgtactacctccagaatggccgagacatgtacgttgaccaggagctcgacatcaaccgactctccgactacgacgtcgaccacattgttcctcagtccttcctcaaggacgactccatcgacaacaaggttctgacccgatctgacaagaaccgaggtaagtccgacaacgttccctccgaagaggtcgttaagaagatgaagaactactggcgacagcttctcaacgccaaactgatcacccagcgaaagtttgacaacctcaccaaggccgagcgaggtggtctgtccgagctggacaaggccggcttcattaagcgacagctggtcgagactcgacagatcaccaagcacgtcgcccagatcctcgactcccgaatgaacaccaagtacgacgagaacgacaagctcatccgggaggtcaaggtcatcaccctgaagtctaagcttgtctccgacttccgaaaggacttccagttctacaaggtccgagagatcaacaactaccaccacgcccacgacgcctacctcaacgccgttgttggtaccgccctcatcaagaagtatcccaagctcgagtccgagttcgtttacggcgactacaaggtttacgatgtccgaaagatgattgccaagtccgagcaggagatcggtaaggccaccgccaagtactttttctactccaacatcatgaatttcttcaagaccgagatcactctcgccaacggtgagattcgaaagcgacccctgattgagactaatggtgagactggtgagatcgtctgggataagggccgagacttcgccaccgtccgaaaggtcctgtccatgccccaggtcaacattgtcaagaagaccgaggtccagaccggtggcttctccaaggagtccattctccccaagcgaaactccgacaaactcatcgcccgtaagaaggactgggatccgaagaagtacggtggtttcgattctcccaccgttgcctactccgtcctcgttgttgctaaagtcgagaagggtaagtctaagaaactcaagtccgtgaaggagctactcggtatcaccatcatggagcgatcttcttttgagaagaaccccattgacttcctcgaggccaagggttacaaagaggtcaagaaggacctgattatcaagctgcccaagtactccctctttgagctcgagaacggccgaaagcgaatgctggcttccgctggtgagctgcagaagggcaacgagctcgctctgccctccaagtacgtcaacttcctctacctggcctcccactacgagaagctcaagggctcccccgaggacaacgagcagaagcagctgttcgttgagcagcacaagcactacctcgacgagatcatcgagcagatctccgagttctccaagcgagtcatcctcgctgacgccaaccttgataaggttctctctgcttacaacaagcaccgggacaagcccatccgagagcaggccgagaatatcatccacctcttcactctcaccaacctcggcgctcctgctgccttcaagtacttcgacaccaccattgaccgaaagaggtacacctccaccaaggaagtcctcgacgccaccctgatccaccagtccatcaccggcctctacgaaacccgaatcgacctctcccagctcggcggtgactctcgagccgaccccaagaagaagcgaaaagtctaaatatccgaagatcaagagcgaagcaagttgtaagtccaggacatgtttcccgcccacgcgagtgatttataacacctctcttttttgacacccgctcgccttgaaattcatgtcacataaattatagtcaacgacgtttgaataacttgtcttgtagttcgatgatgatcatatgattacattaatagtaattactgtatttgatatatatactaattacaatagtacatattagaacatacaatagttagtgccgtgaagtggcttaaaataccgcgagtcgattacgtaatattattacctcttgcccatcgaacgtacaagtactcctctgttctctccttcctttgctttgtgcacgaagaactgcggtcaggtgacacaactttttccatctcagggtgtgtcgcgtgtgcttcatccaaactttagttggggttcgggttcgcgcgagatgatcacgtgccctgatttggtgtcgtcccccgtcgcgctgcgcacgtgatttatttatttccggtggctgctgtctacgcggggccttctctgcccttctgtttcaaccttcgggcggttctcgtaaccagcagtagcaatccatttcgaaactcaaagagctaaaaacgttaaacctcagcagtcgctcgacgaatgggctgcggttgggaagcccacgaggcctatagccagagcctcgagttgacaggagcccagacgccttttccaacggcaacttttatataaaatggcaatgtattcatgcaattgcggccgtgtcaggttggagacactggaccacactctccattgcttcctgaggagatggatcattgctagtgcatctacgcgcagcaatcccgcaagctcgacaaccgtagatgggctttggtgggccaatcaattacgcaacccgcacgttaaattgtatgaggaaggaaggccacggtacaaagtgggtggtcttcacccagtggttgttggtggcgtcatgcagaccatgcattggggatagcacagggttggggtgtcttgtggactcaatgggtgaaaggagatggaaaagggcggtgaaaagtggtagaatcgaaatccctgacgtcaatttataaagtaaaatgcgtttctgccattttgctcccctccttctttcgcaatcgcctccccaaaagttgtcgtggcagtacacatgcttgcatacaatgaagctaatccggcttgctcagtagttgctatatccaggcatggtgtgaaacccctcaaagtatatataggagcggtgagccccagtctggggtcttttctctccatctcaaaactactttctcacaatggccgccctgatgagtccgtgaggacgaaacgagtaagctcgtcggcggctagccacagcgagagttttagagctagaaatagcaagttaaaataaggctagtccgttatcaacttgaaaaagtggcaccgagtcggtgcttttggccggcatggtcccagcctcctcgctggcgccggctgggcaacatgcttcggcatggcgaatgggactaaacttcgagctaatccagtagcttacgttacccaggggcaggtcaactggctagccacgagtctgtcccaggtcgcaatttagtgtaataaacaatatatatattgagtctaaagggaattgtagctattgtgattgtgtgattttcgtcttgctggttcttattgtgtcccattcgtttcatcctgatgaggacccctggaaccggtgttttcttagtctctgcaatcgctagtcttgttgctatgacagttgcgtcgacactattcaggtcatctatcggttattctgatattataatacctccggatcgatgtacctgatttatacttgcagcaatgtttacttcttatcgcgatacacgaatgtgatacggatcaaagtaagcaggactacgataagataacgaatgcggtgcagtccatgtcgattaggtatagatacatttattttgtgttatgttacattttggggggatactgtcctacttgtagtacctacttgtagtggcgcgtctattcctttgccctcggacgagtgctggggcgtcggtttccactatcggcgagtacttctacacagccatcggtccagacggccgcgcttctgcgggcgatttgtgtacgcccgacagtcccggctccggatcggacgattgcgtcgcatcgaccctgcgcccaagctgcatcatcgaaattgccgtcaaccaagctctgatagagttggtcaagaccaatgcggagcatatacgcccggagccgcggcgatcctgcaagctccggatgcctccgctcgaagtagcgcgtctgctgctccatacaagccaaccacggcctccagaagaagatgttggcgacctcgtattgggaatccccgaacatcgcctcgctccagtcaatgaccgctgttatgcggccattgtccgtcaggacattgttggagccgaaatccgcgtgcacgaggtgccggacttcggggcagtcctcggcccaaagcatcagctcatcgagagcctgcgcgacggacgcactgacggtgtcgtccatcacagtttgccagtgatacacatggggatcagcaatcgcgcatatgaaatcacgccatgtagtgtattgaccgattccttgcggtccgaatgggccgaacccgctcgtctggctaagatcggccgcagcgatcgcatccatggcctccgcgaccggctgcagaacagcgggcagttcggtttcaggcaggtcttgcaacgtgacaccctgtgcacggcgggagatgcaataggtcaggctctcgctgaattccccaatgtcaagcacttccggaatcgggagcgcggccgatgcaaagtgccgataaacataacgatctttgtagaaaccatcggcgcagctatttacccgcaggacatatccacgccctcctacatcgaagctgaaagcacgagattcttcgccctccgagagctgcatcaggtcggagacgctgtcgaacttttcgatcagaaacttctcgacagacgtcgcggtgagttcaggctttttcatatgggtacctgagaacatttttgtgtctaggtgtttgtgtttggactgcgatcagtgaagaaaagaagaggaaaaattgtgcaagaaattttgctttcaagacttggctgatgcagcagggtaactctgggacacagacctatgtttgtggttaaactcaatgcacgtggtacgtgcgtggagcgcttacccatccaagggtgtggacatggaaccgacggtccgtggagttgtgtaatgtcattttggcgactcttgaagcaaggctataaaaaaattgtgtggcttgagtcttatcgagctcggtcactacaagagttaatcttcctgtctcaggcagacaggtcaggcagggttacttttgggtgtgctgtaactcactgtatggccgttagtgcgcatagacgttgtacatactggaccgaattgtagcgtgctcaatagggccaataaagctattgtagggatccgaattttcagaacctaatttatctgttacccggcctgtggctcgcacagcttaaaaatggtcaaactttccccttcttgtctttttttcctcacattcatcaggttcttgtcttgatctttcaagtgagtattaattaccgaccttggttcttcattgggagagcattggaagccgtggtgcagcaaccacaaaacggttcttccccttcgataccttcttgcctgcctttcaatacaagtcggctcgattagcggtggtcgcccccgccagcggagaacatggaactaacccagaatgagagctaagtggagaaagaagagagtcagacgactcaagcgaaagcgccgcaaggtccgagctcgatccaaataagcggtttttaacggagatttaacactaaatcgaagaacttttcccgtttcatttgcgaatgagctcgttaacaaaatcccccagtttttttatccagctgtaaggattgacattagtaatgaattattgtttggtatatttaaatctgtagttcctttctgtccgtgtcggcaactgtcgtactcgtgatttacttgtattgacgaatacttactgtagcgcactctgctgctactggtcgtaaggatgtgctatttcggtgtatggtgggttttttgggggtcggaaccgaagactgttacacgggcacggctcgttgtgtacacgcacagagctcttgcgagtcatgttgtagctagctcgtcgtgttcaggaactgttcgatggttcggagagagtcgccgcccagaacatacgcgcaccgatgtcagcagacagccttattacaagtatattcaagcaagtatatccgtagggtgcgggtgatttggatctaaggttcgtactcaacactcacgagcagcttgcctatgttacatccttttatcagacataacataattggagtttacttacacacggggtgtacctgtatgagcaccacctacaattgtagcactggtacttgtacaaagaatttattcgtacgaatcacagggacggccgccctcaccgaaccagcgaatacctcagcggtcccctgcagtgactcaacaaagcgatatgaacatcttgcgatggtatcctgctgatagtttttactgtacaaacacctgtgtagctccttctagcatttttaagttattcacacctcaaggggagggataaattaaataaattccaaaagcgaagatcgagaaactaaattaaaattccaaaaacgaagttggaacacaaccccccgaaaaaaaacaacaaacaaaaaacccaacaaaataaacaaaaacaaaataaatatataactaccagtatctgactaaaagttcaaatactcgtacttacaacaaatagaaatgagccggccaaaattctgcagaaaaaaatttcaaacaagtactggtataattaaattaaaaaacacatcaaagtatcataacgttagttattttattttatttaataaaagaaaacaacaagatgggctcaaaactttcaacttatacgatacataccaaataacaatttagtatttatctaagtgcttttcgtagataatggaatacaaatggatatccagagtatacacatggatagtatacactgacacgacaattctgtatctctttatgttaactactgtgaggcattaaatagagcttgatatataaaatgttacatttcacagtctgaacttttgcagattacctaatttggtaagatattaattatgaactgaaagttgatggcatccctaaatttgatgaaagatgaaattgtaaatgaggtggtaaaagagctacagtcgttttgttttgagataccatcatctctaacgaaatatctattaaaaatctcagtgtgatcatgagtcattgccatcctggaaaatgtcatcatggctgatatttctaactgtttacttgagataaatatatatttacaagaacttcccttgaaattaatttagatataaaatgtttgcgggcaagttactacgaggaataaattatatctaga

SEQ ID NO: 9 - DNA, 12810 bp, synthetic construct
  source 1..12810: mol_type = other DNA
ggttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctgcaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaacacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctggcctaggaagcgacttccaatcgctttgcatatccagtaccacacccacaggcgtttgtgctactctactgatagcaatagatgcgtcataattggttggcccgctgagcctccacaggatactattgcacataccctggtcatgtgcagatcagctcatttgtggagactctggagtaacttagacgacgcctggttcaattgccgcaatgtgcgcccacgcagataatgtattgaggggtggagcgcctcttggggacttgctgtacttgtacgggatattaaacgcactcagcaagaccatgacgtaaaacacacctactgtacgatacgtactgtaggtattgtactcgtacccggtactacaaatagtacgatactatacggagtgtatttgtaccttgatatacgactggcggagtgaagagaaggagttgaacaagaccagatggggatatcagccccagtgctttgtattacaagtacgagtacttaatagatactgtaaggctattgatacggatggcagtaagtcattgagtaagcaattgtggcccagcatctcccctacgtacttgtaccataccccatggagacaccaatggtctttcacgcacactgtcgtgtgctgtatcgcagaatcgggtgtccaaccaaatgccgttacccccacgtcacagccgatagacagatacaccatcaataccagcaggttgtatcatgcggttggctgaaggtaagctgattggtctaaaaactgtagctgtcctaattcaacgagcgctatttggggccaaccacctcggccaagcggcctttaatctgcgtgccccagaggcgtctaatgaggctctggccgccactgtaggagtgtttctctgtgcgcacacgcagttttgagtttgggcgactttccctttttcccaattgcgtacacacacagctccgagctaagcgctgtccttgaaccttctccctcttttccctctttttctcttccccttcccctcctccacattaaggccaaatcctgaattgcaccaactagtacaacgacaacaatggacaagaagtactccatcggtttggacattggtactaactctgtcggctgggccgtcatcaccgacgagtacaaggttccctccaagaagttcaaggtccttggcaacaccgaccgacactctatcaagaagaacctgatcggtgctctgctgttcgactctggcgagactgccgaggccacccgactgaagcgaaccgctcgacgccgatacacccgacgaaagaaccgaatctgttacctccaggagatcttcagcaacgagatggctaaggtcgacgactccttcttccaccgactcgaggagtctttcctggtcgaagaggataagaagcacgagcgacaccccatcttcggcaacattgttgatgaggttgcctaccatgagaagtaccccaccatctaccacctccgaaagaagctcgtcgactccactgacaaggctgacctccgactcatctaccttgctctcgcccacatgatcaagttccgaggtcacttcctcattgagggtgatctcaaccccgacaactccgacgttgacaagctgttcatccagctcgtccagacctacaaccagctctttgaggagaaccctatcaacgcttctggtgttgacgccaaggccattctctccgcccgactctctaagtcccgacgactcgagaacctcattgcccagctgcccggcgagaagaagaacggcctcttcggtaacctgattgctctctctcttggtctgacccccaacttcaagtccaactttgacctcgccgaggacgccaagctccagctgtccaaggacacctacgatgacgatctggacaacctcctggcccagatcggtgaccagtacgccgatctcttccttgccgccaagaacctctccgacgccatcctgctctccgacatcctccgagtcaacaccgagattaccaaggctcctctgtctgcctctatgatcaagcgatacgacgagcaccaccaggatctcactcttctcaaggctctcgtccgacagcagctccccgagaagtacaaggagattttctttgaccagtccaagaacggttacgctggctacattgacggtggtgcttcccaggaagagttttacaagttcatcaagcctattctggagaagatggacggtaccgaggagctgctcgtcaagctcaaccgagaggacctccttcgaaagcagcgaaccttcgataacggctccatcccccaccagatccacctgggtgagctccacgccattctccgaagacaagaggacttctaccccttcctaaaggataaccgagagaagatcgagaagattctcaccttccgaatcccctactacgtcggtcccctcgctcgaggtaactcccgatttgcttggatgacccgaaagtccgaggagactatcaccccctggaactttgaagaggtagtcgacaagggtgcctccgcccagtctttcattgagcggatgaccaacttcgataagaacctccccaacgagaaggtccttcccaagcactctctcctctacgagtacttcaccgtctacaacgagctgaccaaggtcaagtacgttaccgagggcatgcgaaagcccgctttcctctctggtgagcagaagaaggccattgtcgacctcctgttcaagactaaccgaaaagtcaccgtcaagcagctcaaggaagactacttcaagaagattgagtgcttcgactccgtcgagatttccggtgtcgaggaccgattcaacgcctccctcggcacctaccacgatcttctgaagatcatcaaggacaaggactttcttgataacgaggagaacgaggacattctcgaggacatcgtcctcaccctcacccttttcgaggatcgagagatgatcgaggagcgactcaagacctacgcccatctcttcgacgacaaggtcatgaagcaactcaagcgacgacgatacactggctggggccgactttcccgaaagctcatcaacggcatccgagacaagcagtctggcaagaccatcctggacttcctgaagtccgacggtttcgccaaccgaaacttcatgcagctcatccacgacgactctcttaccttcaaagaggatatccagaaggcccaggtttctggccagggcgactccctccacgagcacattgccaacctcgccggatcccccgccatcaaaaagggtatcctccagaccgtcaaggttgtcgacgaactcgtgaaggtcatgggccgacacaagcccgagaacatcgttatcgagatggcccgagagaaccagaccacccagaagggtcagaagaactcccgagagcgaatgaagcgaatcgaagagggtatcaaggagctcggttcccagattctcaaggagcaccccgtcgagaacacccagctccagaacgagaaactctacctgtactacctccagaatggccgagacatgtacgttgaccaggagctcgacatcaaccgactctccgactacgacgtcgaccacattgttcctcagtccttcctcaaggacgactccatcgacaacaaggttctgacccgatctgacaagaaccgaggtaagtccgacaacgttccctccgaagaggtcgttaagaagatgaagaactactggcgacagcttctcaacgccaaactgatcacccagcgaaagtttgacaacctcaccaaggccgagcgaggtggtctgtccgagctggacaaggccggcttcattaagcgacagctggtcgagactcgacagatcaccaagcacgtcgcccagatcctcgactcccgaatgaacaccaagtacgacgagaacgacaagctcatccgggaggtcaaggtcatcaccctgaagtctaagcttgtctccgacttccgaaaggacttccagttctacaaggtccgagagatcaacaactaccaccacgcccacgacgcctacctcaacgccgttgttggtaccgccctcatcaagaagtatcccaagctcgagtccgagttcgtttacggcgactacaaggtttacgatgtccgaaagatgattgccaagtccgagcaggagatcggtaaggccaccgccaagtactttttctactccaacatcatgaatttcttcaagaccgagatcactctcgccaacggtgagattcgaaagcgacccctgattgagactaatggtgagactggtgagatcgtctgggataagggccgagacttcgccaccgtccgaaaggtcctgtccatgccccaggtcaacattgtcaagaagaccgaggtccagaccggtggcttctccaaggagtccattctccccaagcgaaactccgacaaactcatcgcccgtaagaaggactgggatccgaagaagtacggtggtttcgattctcccaccgttgcctactccgtcctcgttgttgctaaagtcgagaagggtaagtctaagaaactcaagtccgtgaaggagctactcggtatcaccatcatggagcgatcttcttttgagaagaaccccattgacttcctcgaggccaagggttacaaagaggtcaagaaggacctgattatcaagctgcccaagtactccctctttgagctcgagaacggccgaaagcgaatgctggcttccgctggtgagctgcagaagggcaacgagctcgctctgccctccaagtacgtcaacttcctctacctggcctcccactacgagaagctcaagggctcccccgaggacaacgagcagaagcagctgttcgttgagcagcacaagcactacctcgacgagatcatcgagcagatctccgagttctccaagcgagtcatcctcgctgacgccaaccttgataaggttctctctgcttacaacaagcaccgggacaagcccatccgagagcaggccgagaatatcatccacctcttcactctcaccaacctcggcgctcctgctgccttcaagtacttcgacaccaccattgaccgaaagaggtacacctccaccaaggaagtcctcgacgccaccctgatccaccagtccatcaccggcctctacgaaacccgaatcgacctctcccagctcggcggtgactctcgagccgaccccaagaagaagcgaaaagtctaaatatccgaagatcaagagcgaagcaagttgtaagtccaggacatgtttcccgcccacgcgagtgatttataacacctctcttttttgacacccgctcgccttgaaattcatgtcacataaattatagtcaacgacgtttgaataacttgtcttgtagttcgatgatgatcatatgattacattaatagtaattactgtatttgatatatatactaattacaatagtacatattagaacatacaatagttagtgccgtgaagtggcttaaaataccgcgagtcgattacgtaatattattacctcttgcccatcgaacgtacaagtactcctctgttctctccttcctttgctttgtgcacgaagaactgcggtcaggtgacacaactttttccatctcagggtgtgtcgcgtgtgcttcatccaaactttagttggggttcgggttcgcgcgagatgatcacgtgccctgatttggtgtcgtcccccgtcgcgctgcgcacgtgatttatttatttccggtggctgctgtctacgcggggccttctctgcccttctgtttcaaccttcgggcggttctcgtaaccagcagtagcaatccatttcgaaactcaaagagctaaaaacgttaaacctcagcagtcgctcgacgaatgggctgcggttgggaagcccacgaggcctatagccagagcctcgagttgacaggagcccagacgccttttccaacggcaacttttatataaaatggcaatgtattcatgcaattgcggccgtgtcaggttggagacactggaccacactctccattgcttcctgaggagatggatcattgctagtgcatctacgcgcagcaatcccgcaagctcgacaaccgtagatgggctttggtgggccaatcaattacgcaacccgcacgttaaattgtatgaggaaggaaggccacggtacaaagtgggtggtcttcacccagtggttgttggtggcgtcatgcagaccatgcattggggatagcacagggttggggtgtcttgtggactcaatgggtgaaaggagatggaaaagggcggtgaaaagtggtagaatcgaaatccctgacgtcaatttataaagtaaaatgcgtttctgccattttgctcccctccttctttcgcaatcgcctccccaaaagttgtcgtggcagtacacatgcttgcatacaatgaagctaatccggcttgctcagtagttgctatatccaggcatggtgtgaaacccctcaaagtatatataggagcggtgagccccagtctggggtcttttctctccatctcaaaactactttctcacaatgctgcatctgatgagtccgtgaggacgaaacgagtaagctcgtcatgcagctcttcaacgacgcgttttagagctagaaatagcaagttaaaataaggctagtccgttatcaacttgaaaaagtggcaccgagtcggtgcttttggccggcatggtcccagcctcctcgctggcgccggctgggcaacatgcttcggcatggcgaatgggactaaacttcgagctaatccagtagcttacgttacccaggggcaggtcaactggctagccacgagtctgtcccaggtcgcaatttagtgtaataaacaatatatatattgagtctaaagggaattgtagctattgtgattgtgtgattttcgtcttgctggttcttattgtgtcccattcgtttcatcctgatgaggacccctggaaccggtgttttcttagtctctgcaatcgctagtcttgttgctatgacagttgcgtcgacactattcaggtcatctatcggttattctgatattataatacctccggatcgatgtacctgatttatacttgcagcaatgtttacttcttatcgcgatacacgaatgtgatacggatcaaagtaagcaggactacgataagataacgaatgcggtgcagtccatgtcgattaggtatagatacatttattttgtgttatgttacattttggggggatactgtcctacttgtagtacctacttgtagtggcgcgtctattcctttgccctcggacgagtgctggggcgtcggtttccactatcggcgagtacttctacacagccatcggtccagacggccgcgcttctgcgggcgatttgtgtacgcccgacagtcccggctccggatcggacgattgcgtcgcatcgaccctgcgcccaagctgcatcatcgaaattgccgtcaaccaagctctgatagagttggtcaagaccaatgcggagcatatacgcccggagccgcggcgatcctgcaagctccggatgcctccgctcgaagtagcgcgtctgctgctccatacaagccaaccacggcctccagaagaagatgttggcgacctcgtattgggaatccccgaacatcgcctcgctccagtcaatgaccgctgttatgcggccattgtccgtcaggacattgttggagccgaaatccgcgtgcacgaggtgccggacttcggggcagtcctcggcccaaagcatcagctcatcgagagcctgcgcgacggacgcactgacggtgtcgtccatcacagtttgccagtgatacacatggggatcagcaatcgcgcatatgaaatcacgccatgtagtgtattgaccgattccttgcggtccgaatgggccgaacccgctcgtctggctaagatcggccgcagcgatcgcatccatggcctccgcgaccggctgcagaacagcgggcagttcggtttcaggcaggtcttgcaacgtgacaccctgtgcacggcgggagatgcaataggtcaggctctcgctgaattccccaatgtcaagcacttccggaatcgggagcgcggccgatgcaaagtgccgataaacataacgatctttgtagaaaccatcggcgcagctatttacccgcaggacatatccacgccctcctacatcgaagctgaaagcacgagattcttcgccctccgagagctgcatcaggtcggagacgctgtcgaacttttcgatcagaaacttctcgacagacgtcgcggtgagttcaggctttttcatatgggtacctgagaacatttttgtgtctaggtgtttgtgtttggactgcgatcagtgaagaaaagaagaggaaaaattgtgcaagaaattttgctttcaagacttggctgatgcagcagggtaactctgggacacagacctatgtttgtggttaaactcaatgcacgtggtacgtgcgtggagcgcttacccatccaagggtgtggacatggaaccgacggtccgtggagttgtgtaatgtcattttggcgactcttgaagcaaggctataaaaaaattgtgtggcttgagtcttatcgagctcggtcactacaagagttaatcttcctgtctcaggcagacaggtcaggcagggttacttttgggtgtgctgtaactcactgtatggccgttagtgcgcatagacgttgtacatactggaccgaattgtagcgtgctcaatagggccaataaagctattgtagggatccgaattttcagaacctaatttatctgttacccggcctgtggctcgcacagcttaaaaatggtcaaactttccccttcttgtctttttttcctcacattcatcaggttcttgtcttgatctttcaagtgagtattaattaccgaccttggttcttcattgggagagcattggaagccgtggtgcagcaaccacaaaacggttcttccccttcgataccttcttgcctgcctttcaatacaagtcggctcgattagcggtggtcgcccccgccagcggagaacatggaactaacccagaatgagagctaagtggagaaagaagagagtcagacgactcaagcgaaagcgccgcaaggtccgagctcgatccaaataagcggtttttaacggagatttaacactaaatcgaagaacttttcccgtttcatttgcgaatgagctcgttaacaaaatcccccagtttttttatccagctgtaaggattgacattagtaatgaattattgtttggtatatttaaatctgtagttcctttctgtccgtgtcggcaactgtcgtactcgtgatttacttgtattgacgaatacttactgtagcgcactctgctgctactggtcgtaaggatgtgctatttcggtgtatggtgggttttttgggggtcggaaccgaagactgttacacgggcacggctcgttgtgtacacgcacagagctcttgcgagtcatgttgtagctagctcgtcgtgttcaggaactgttcgatggttcggagagagtcgccgcccagaacatacgcgcaccgatgtcagcagacagccttattacaagtatattcaagcaagtatatccgtagggtgcgggtgatttggatctaaggttcgtactcaacactcacgagcagcttgcctatgttacatccttttatcagacataacataattggagtttacttacacacggggtgtacctgtatgagcaccacctacaattgtagcactggtacttgtacaaagaatttattcgtacgaatcacagggacggccgccctcaccgaaccagcgaatacctcagcggtcccctgcagtgactcaacaaagcgatatgaacatcttgcgatggtatcctgctgatagtttttactgtacaaacacctgtgtagctccttctagcatttttaagttattcacacctcaaggggagggataaattaaataaattccaaaagcgaagatcgagaaactaaattaaaattccaaaaacgaagttggaacacaaccccccgaaaaaaaacaacaaacaaaaaacccaacaaaataaacaaaaacaaaataaatatataactaccagtatctgactaaaagttcaaatactcgtacttacaacaaatagaaatgagccggccaaaattctgcagaaaaaaatttcaaacaagtactggtataattaaattaaaaaacacatcaaagtatcataacgttagttattttattttatttaataaaagaaaacaacaagatgggctcaaaactttcaacttatacgatacataccaaataacaatttagtatttatctaagtgcttttcgtagataatggaatacaaatggatatccagagtatacacatggatagtatacactgacacgacaattctgtatctctttatgttaactactgtgaggcattaaatagagcttgatatataaaatgttacatttcacagtctgaacttttgcagattacctaatttggtaagatattaattatgaactgaaagttgatggcatccctaaatttgatgaaagatgaaattgtaaatgaggtggtaaaagagctacagtcgttttgttttgagataccatcatctctaacgaaatatctattaaaaatctcagtgtgatcatgagtcattgccatcctggaaaatgtcatcatggctgatatttctaactgtttacttgagataaatatatatttacaagaacttcccttgaaattaatttagatataaaatgtttgcgggcaagttactacgaggaataaattatatctaga

(19) United States
(12) Patent Application Publication
(10) Pub. No.: US 2025/0109422 A1
(43) Pub. Date: Apr. 3, 2025

(54) YARROWIA PRODUCTION PROCESS

(71) Applicant: DSM IP ASSETS B.V., Heerlen (NL)

(72) Inventors: Peter Louis HOUSTON, Kaiseraugst (CH); Maria Elena MAYORGA, Kaiseraugst (CH); Valmik Kanubhai VYAS, Kaiseraugst (CH); Peter Scott YORGEY, Kaiseraugst (CH)

(21) Appl. No.: 18/834,722

(22) PCT Filed: Feb. 1, 2023
(86) PCT No.: PCT/EP2023/052376

§ 371 Date: Jul. 31, 2024

Related U.S. Application Data
(60) Provisional application No. 63/305,852, filed on Feb. 2, 2022.

(51) Int. Cl. C12P 23/00; C12N 1/06; C12N 9/10; C12N 15/81

(52) U.S. Cl. CPC C12P 23/00; C12N 1/063; C12N 9/1051; C12N 15/815; C12Y 204/01232

(57) ABSTRACT

The present invention is related to a novel process on biological production of lipophilic substances in an oleaginous host cell, particularly Yarrowia lipolytica, comprising modification in the N-glycosylation pathway, leading to higher yield and product purity with further facilitating the overall production process.

The present invention is related to a novel process on biological production of lipophilic substances in an oleaginous host cell, particularly Yarrowia lipolytica, comprising modification in the N-glycosylation pathway, leading to higher yield and product purity with further facilitating the overall production process.

For production of lipophilic substances, oleaginous cells can be used, such as e.g. cells of Yarrowia lipolytica. However, efficient extraction of intracellular accumulated lipophilic substances from yeast is challenging because of the thick and rigid cell wall. Due to the differences within the yeast species, i.e. different physical properties and structures of the cell walls, there is no universal method available that can be uniformly used for all host organisms.

Methods for disruption of cells in general include mechanical and non-mechanical methods, such as e.g. ultrasonic, microwave, high-pressure homogenization, bead milling, grinding, enzymatic or chemical digestion or lysis of the cells. All these extraction methods are cost and energy-intensive and thus should be minimized.

Thus, there is a strong need to find a proper eco-friendly and energy-saving solution for extraction of lipophilic substances from oleaginous host cells, such as Yarrowia lipolytica, wherein the biochemical properties of said products that are intracellular accumulated are not extremely changed, including the use of “green” extraction solvents.

Surprisingly, we now found a way to enhance extractability of lipophilic substances that are accumulated within the host cell, i.e. intracellularly, during fermentation via modification of certain endogenous genes involved in N-glycosylation pathway and or cell wall integrity of the host cell leading to enhanced solvent extraction of said accumulated lipophilic substances without compromising the cell-integrity and growth performance during the fermentation process.

Particularly, the present invention is directed to genetically modified host cell, particularly oleaginous yeast, such as Yarrowia lipolytica, capable of accumulating intracellular lipophilic substances such as fat-soluble vitamins, carotenoids or polyunsaturated fatty acids (PUFAs), said host cell comprising a mutation in the N-glycosylation pathway or in genes involved in cell wall integrity, preferably comprising genetic modification in endogenous genes encoding mannosyltransferases of the mannan polymerase complex.

The present invention is furthermore directed to production of lipophilic substances as defined herein using such genetically modified host cell as defined herein, wherein the percentage of said lipophilic substances, preferably carotenoids, recovered/extracted from the production medium could be increased in the range of at least 2 to 10%, such as e.g. 2, 5, 7, 10, 15, 20, 25, 30, 35, 40% and more.

The present invention is furthermore directed to extraction of lipophilic substances, preferably carotenoids, as produced in a genetically modified host cell as defined herein and under conditions as defined herein, said extraction comprising mechanical and/or non-mechanical disruption of the cell wall, wherein the recovery steps including physical or (bio)chemical cell lysis could be reduced or even abolished compared to a process using the respective non-modified host cell.

The present invention is furthermore directed to extraction of lipophilic substances as produced in a genetically modified host cell as defined herein and under conditions as defined herein, wherein the amount/percentage of lipids being extracted is increased, including increasing the amount/percentage of lipophilic substances, particularly carotenoids, is increased without compromising the growth and/or yield of the host cell towards said lipophilic substance, i.e. the fermentation product of interest, particularly carotenoid.

Particularly, the product/lipophilic substance of interest, such as e.g. carotenoid, fat-soluble vitamin or polyunsaturated fatty acids, preferably carotenoids, within the lipid fraction can be increased by at least 2 to 10% using enzymatic treatment as defined herein at the end of the fermentation with a genetically modified host cell according to all embodiments of the present invention followed by extraction of the lipophilic substance using solvents, such as e.g. hexane, acetone or the like.

The present invention is furthermore directed to extraction of lipophilic substances as produced in a genetically modified host cell as defined herein and under conditions as defined herein, said extraction comprising the use of cell-lysing enzymes wherein the concentration of said enzymes could be reduced compared to a process using the respective non-modified host cell.

The present invention is furthermore directed to extraction of lipophilic substances, preferably carotenoids, as produced in a genetically modified host cell as defined herein and under conditions as defined herein, said extraction comprising the use of mechanical cell lysis, particularly bead milling, wherein the passes of bead milling could be reduced compared to a process using the respective non-modified host cell, such as reduced to 5 or less passes, preferably less than 4, 3, 2, or even 1 pass, most preferably wherein the mechanical lysis including e.g. the use of bead milling can be abolished.

In a particular embodiment, the present invention is directed to production of lipophilic substances, preferably carotenoids, as defined herein in a genetical modified host cell, particularly oleaginous yeast, such as Yarrowia lipolytica, as defined herein, comprising bead milling, wherein the extractability of the lipophilic substance, preferably carotenoids, could be increased to at least about 45%, e.g. a range of 45 to 80%, such as e.g. at least about 50, 55, 60, 65, 70, 75, 80% or even more, particularly wherein the % of lipophilic substances, preferably carotenoids, more preferably astaxanthin, based on total lipophilic substances, preferably carotenoids, more preferably astaxanthin, is in the range of 45 to 80% and wherein the extraction is performed in a solvent comprising acetone.

In a particular embodiment, the present invention is directed to production of lipophilic substances, preferably carotenoids, as defined herein in a genetical modified host cell, particularly oleaginous yeast, such as Yarrowia lipolytica, as defined herein, comprising bead milling, wherein the extractability of the lipophilic substance, preferably carotenoids, could be increased to at least about 70%, such as e.g. a range of 70 to 80% or more, after 1 pass of bead milling as compared to a non-modified host cell, particularly wherein the % of lipophilic substances, preferably carotenoids, more preferably astaxanthin, based on total lipophilic substances, preferably carotenoids, more preferably astaxanthin, even more preferably diacetyl astaxanthin, is in the range of 70 to 80% after 1 pass of bead milling and wherein the extraction is performed in a solvent comprising acetone.

In a particular embodiment, the present invention is directed to production of lipophilic substances, preferably carotenoids, as defined herein in a genetical modified host cell, particularly oleaginous yeast, such as Yarrowia lipolytica, as defined herein, comprising bead milling, wherein the extractability of the lipophilic substance, preferably carotenoids, could be increased to at least about 80%, after 2 passes of bead milling as compared to a non-modified host cell, particularly wherein the % of lipophilic substances, preferably carotenoids, more preferably astaxanthin, based on total lipophilic substances, preferably carotenoids, more preferably astaxanthin, is in the range of at least 80% after 2 passes of bead milling and wherein the extraction is performed in a solvent comprising acetone.

In a particular embodiment, the present invention is directed to production of lipophilic substances, preferably carotenoids, as defined herein in a genetical modified host cell, particularly oleaginous yeast, such as Yarrowia lipolytica, as defined herein, comprising application of cell-lysing enzymes, wherein the percentage of lipophilic substances, preferably carotenoids, within the lipid fraction could be increased after enzyme treatment compared to a process using the respective non-modified host cell.

Thus, in one preferred embodiment, the present invention is directed to production of lipophilic substances, preferably carotenoids, more preferably astaxanthin, is a genetically modified host cell, particularly oleaginous yeast, such as Yarrowia lipolytica, as defined herein, comprising application of cell-lysing enzymes, and wherein the cell rupture could be increased by at least about 50%, such as e.g. in a range of 50 to 70%, after 1 pass of bead milling, and preferably wherein the cell rupture is increased to at least about 70% after 2 passes of bead milling.

Suitable host cells in accordance with all embodiments of the present invention might be any host cell capable of accumulating lipophilic substances. Preferably, a suitable host cell is selected from oleaginous yeast, such as e.g. Yarrowia, particularly Yarrowia lipolytica, said host cell comprising a mutation in one or more genes involved in the endogenous N-glycosylation pathway, particularly within enzymes of the mannan polymerase complex and/or in genes involved in cell wall integrity.

The term “oleaginous”, particularly “oleaginous yeast” refers to the ability of the host cell to accumulate at least about 20% of its dry cell weight as lipid, such as e.g. a range of 20-45% of its dry cell weight as lipid, as particularly defined in U.S. Pat. No. 9,297,031. Such oleaginous yeast can be naturally accumulating said amount of lipids or can be genetically manipulated or modified to accumulate such percentage of lipids. A non-limiting list of such oleaginous host cells according to the present invention include strains of Yarrowia, Saccharomyces, Candida, Trichosporon, Rhodotorula, Rhodosporidium, Cryptococcus, Lipomyces, Blakeslea, Fusarium, Klyveromyces, particularly Yarrowia lipolytica, Saccharomyces cerevisiae, Candida utilis, Blakeslea trispora, Candida tropicalis, Phaffia rhodozyma, Trichosporum pullulans or cutaneum, Klyveromyces marxianus, Rhodotorula glutinis or graminis, Lipomyces starkeyi, Cryptococcus albidus or curvatus, preferably Yarrowia lipolytica.

The host cell as defined herein is capable of accumulating lipophilic substances. As used herein and in accordance with all embodiments of the present invention, the term “lipophilic substances” might be selected from fat-soluble vitamin, carotenoids, or polyunsaturated fatty acids (PUFAs). Thus, said host cell is preferably genetically modified to accumulate said lipophilic substances, particularly genes involved in biosynthesis of carotenoids, fat-soluble vitamins and/or PUFAs, as e.g. described in WO2016172282, WO2008073367 and as known in the art. Preferably, the host cell as defined herein is a carotenoid-producing host cell, more preferably an astaxanthin-producing host cell

The term “accumulation” in connection with lipophilic substances as defined herein and according to all embodiments of the present invention means the intracellular buildup of biomass associated lipids or such lipophilic substances, said buildup being increased compared to a host cell wherein genes involved in biosynthesis of said lipophilic substances, i.e. fat-soluble vitamins, carotenoids or PUFAs, preferably carotenoids, are not (over)expressed, i.e. the host cell is said to be “wild-type” with regards to said genes. Typically, it means an increase of at least about 1-10%, such as 1 to 5,10-50, 30-80, 20-70% in intracellular buildup as defined above. Thus, a carotenoid-accumulating strain according to this definition comprising a genetically modification as defined herein is a strain that produces at least about 1 to 10 or more g/l of the carotenoids, such as e.g. about 1, 2, 3, 5, 7, 8.10 g/l or more, as compared to the non-carotenoid producing host cell, such as e.g. Yarrowia lipolytica ML13961 producing less than 1 g/l of said carotenoid, typically wherein the non-accumulating strain comprises zero gram of said carotenoids.

Preferably, fat-soluble vitamins according to the present invention are selected from the group consisting of vitamin A, vitamin D, vitamin E, including all metabolites, precursors or derivatives thereof, as long as these vitamins are accumulated intracellularly and they are not produced in a 2-phase-cultivation system comprising accumulation of said lipophilic substances in a so-called second phase. The skilled person will know how to manipulate a suitable host cell as defined herein to produce such fat-soluble vitamins, see e.g. WO2008130372.

Thus, for example, the term “vitamin D” according to all embodiments of the present invention includes but is not limited to vitamin D3, vitamin D2, 7-dehydrocholesterol (7-DHC), 25-OH-vitamin D3 or calcidiol (HyD), calcitriol, 1,25-dihydroxy vitamin D3, ergocalciferol.

Thus, for example, the term “vitamin A” according to all embodiments of the present invention includes but is not limited to 3-OH-retinol, retinal, 3-OH-4-ketoretinol, 3-OH-retinal, 3-OH-4-ketoretinal, 3-OH-retinoic acid, 3-OH-4-ketoretinoic acid.

Thus, for example, the term “vitamin E” according to all embodiments of the present invention includes but is not limited to alpha-, beta-, gamma-, delta-tocopherol, tocotrienols, tocopherol acetate.

Preferably, carotenoids according to the present invention are selected from the group consisting of astaxanthin (AXN), zeaxanthin (ZEA), canthaxanthin (CXN), R-cryptoxanthin, lutein, lycopene, rhodoxanthin, beta-carotene, alpha-carotene, gamma-carotene, including all metabolites, precursors or derivatives thereof, such as e.g. disclosed in WO2003097798, WO2014096990, more preferably selected from AXN, ZEA, CXN, beta-carotene, most preferably AXN.

In general, and as used herein, the term “carotenoid” refers to a class of hydrocarbons having a conjugated polyene carbon skeleton formally derived from isoprene and includes C30 diapocarotenoids and C40 carotenoids and their oxygenated derivatives. It includes both carotenes, such as e.g. phytoene, beta-carotene and lycopene, and xanthophylls, such as e.g. AXN, CXN, cryptoxanthin, ZEA, lutein, e.g. carotenoids oxidized on the 4-keto position or 3-hydroxy position to yield CXN, ZEA, or AXN. Biosynthesis of carotenoids is described in e.g. WO2006102342.

Thus, for example, the term “AXN”, “ZEA”, according to all embodiments of the present invention also includes but is not limited to mono- or di-acetylated forms, e.g. di-acetylated AXN, mono-acetylated AXN, mono-acetylated ZXN, di-acetylated ZEA, and mixtures thereof, such as mixtures comprising 60-80% di-acetylated forms. The skilled person will know how to generate those acetylated forms of carotenoids, such as described in WO2014096992.

Preferably, PUFAs according to the present invention are selected from the group consisting of eicosadienoic acid (EDA; 20:2, n-6), eicosatretaenoic acid (ETA; 20:4, n-3), eicosapentaenoic acid (EPA; C20:5, n-3), docosahexaenoic acid (DHA; C22:6, n-3), docosapentaenoic acid (DPA; C22:5, n-6 or n-3), arachidonic acid (ARA; C20:4, n-6), gamma-linolenic acid (GLA; C18:3, n-6), alpha-linolenic acid (ALA; C18:3, n-3), linoleic acid (LA; C18:2, n-6), stearidonic acid (STA; C18:4, n-3), and combinations thereof. The skilled person will know how to generate those PUFAs including the genes involved in the biosynthetic pathway, see e.g. WO2006052870 or WO2006052871.

Suitable endogenous genes involved in the N-glycosylation pathway to be modified according to all embodiments of the present invention might be selected from genes encoding mannosyltransferases, such as e.g. transferases involved in mannan polymerase complex, including but not limited to one or more enzyme(s) with activity of OCH1, MNN9, VAN1, MNN10 (YALI0_E12199g=XM_503853.1), MNN11 (YALI0_F17402g=XM_505534.1), ANP1 (YALI0_C04004g=XM_501421.1), HOC1, preferably endogenous MNN9, such as MNN9 from Yarrowia lipolytica.

Suitable endogenous genes involved in cell wall integrity to be optionally furthermore modified according to all embodiments of the present invention might be selected from genes encoding synthases, such as e.g. chitin synthases, particularly chitin synthase IV (CHS4), or linking proteins between cell wall glucans and chitin, particularly glycosylphosphatidylinositol-anchored plasma membrane glycoprotein I (GAS1), preferably endogenous CHS4, such as CHS4 from Yarrowia lipolytica.

As defined herein, a “modified host cell” is compared to a “wild-type host cell”, i.e., the respective host cell without such modification in the defined enzyme activities as defined herein, particularly activity of endogenous genes involved in the N-glycosylation pathway, more particularly mannosyltransferases, and/or genes involved in cell wall integrity, i.e. wherein said corresponding endogenous enzyme is (still) expressed and active in vivo. It might also refer to accumulation of lipophilic substances, preferably carotenoids, as defined herein, i.e. wherein the host cell is (over)expression endogenous or heterologous genes involved in biosynthesis of said lipophilic substances, preferably carotenoids, and wherein said gene expression/overexpression is compared to a wild-type host with “regular” or “normal”, i.e. wild-type expression, or no such expression of said heterologous genes which have to be introduced into the host cell.

Suitable enzymes involved in cell lysis according to all embodiments of the present invention are selected from enzymatic compositions comprising proteases, zymolases, chitinases, mannanases, glucanases, alcalases, xylanases, cellulases and/or mixtures thereof, as known to the skilled person. Particularly useful are compositions comprising alcalases, xylanases, glucanases and/or cellulases. For extraction of the lipophilic substances, preferably carotenoids, suitable solvents include compositions comprising dichloromethane, hexane, octanol, acetone, ethyl acetate, isobutyl acetate, wherein preferably dichloromethane is excluded.

In one embodiment, the present invention provides a modified host cell accumulating lipophilic substances, preferably carotenoids, more preferably AXN, as defined herein, comprising a modification in a polypeptide with at least about 50%, such as 60, 70, 80, 90, 95, 98, or 100% identity to SEQ ID NO:1, including but not limited to MNN9 encoded by a polynucleotide according to SEQ ID NO:2 obtainable from Yarrowia lipolytica, wherein the activity of said polypeptide is reduced or abolished, preferably abolished, including reduction or abolishment of gene expression. Particularly, the mannan polymerase complexes subunit MNN9 is characterized e.g. in that its function is disrupted by a 2 bp insertion between nucleotides 184-185 of the ORF encoding the mannan polymerase complexes subunit MNN9 as shown in SEQ ID NO:2, leading to a null-mutant.

In one embodiment, the present invention provides a modified host cell accumulating lipophilic substances, preferably carotenoids, more preferably AXN, as defined herein, comprising a modification in a polypeptide with at least about 50%, such as 60, 70, 80, 90, 95, 98, or 100% identity to SEQ ID NO:3, including but not limited to CHS4 encoded by a polynucleotide according to SEQ ID NO:4 obtainable from Yarrowia lipolytica, wherein the activity of said polypeptide is reduced or abolished, preferably abolished, including reduction or abolishment of gene expression.

In one embodiment, the present invention provides a modified host cell accumulating lipophilic substances, preferably carotenoids, more preferably AXN, as defined herein, comprising a modification in a polypeptide with at least about 50%, such as 60, 70, 80, 90, 95, 98, or 100% identity to SEQ ID NO:5, including but not limited to GAS1 encoded by a polynucleotide according to SEQ ID NO:6 obtainable from Yarrowia lipolytica, wherein the activity of said polypeptide is reduced or abolished, preferably abolished, including reduction or abolishment of gene expression.

In further embodiments, the present invention provides a modified host cell accumulating lipophilic substances as defined herein, comprising a modification in a polypeptide with at least about 50%, such as 60, 70, 80, 90, 95, 98, or 100% identity to a polypeptide of SEQ ID NO:1, including but not limited to MNN9 encoded by a polynucleotide selected from SEQ ID NO:2 obtainable from Yarrowia lipolytica, wherein the activity of said polypeptide is reduced or abolished, preferably abolished, including reduction or abolishment of gene expression, said host cell comprising at least a further genetic modification, such as reduction or abolishment of endogenous genes encoding glucanosyltransferases, particularly a modification in a polypeptide with at least about 50%, such as 60, 70, 80, 90, 95, 98, or 100% identity to a polypeptide selected from SEQ ID NO:5 including GAS1 encoded by a polynucleotide according to SEQ ID NO:6 obtainable from Yarrowia lipolytica, said modified host cell furthermore comprising at least one genetic modification, such as reduction or abolishment of endogenous genes encoding chitin synthase, particularly a modification in a polypeptide with at least about 50%, such as 60, 70, 80, 90, 95, 98, or 100% identity to a polypeptide selected from SEQ ID NO:17 including CHS4 encoded by a polynucleotide according to SEQ ID NO:4 obtainable from Yarrowia lipolytica.

Preferably, the host cell as defined herein comprises a genetic modification leading to reduction or abolishment of the endogenous MNN9 as defined herein, optionally further comprising a genetic modification leading to reduction or abolishment of the endogenous CHS4 as defined herein, said host cell being preferably selected from Yarrowia lipolytica, wherein the lipophilic substance accumulated intracellularly being particularly selected from carotenoids, preferably selected from AXN comprising mono- and/or di-acetylated AXN.

As used herein, “activity” of an enzyme, particularly transferase or synthase activity, including activity of endogenous enzymes as defined herein, is defined as “specific activity” i.e. its catalytic activity, i.e. its ability to catalyze formation of a product from a given substrate, such as e.g. the formation of mannosyltransferases or genes involved in carotenoid-biosynthesis. An enzyme according to the present invention is active, if it performs its catalytic activity in vivo, i.e. within the host cell as defined herein or within a system in the presence of a suitable substrate. The skilled person knows how to measure enzyme activity.

As used herein, an enzyme, particularly a transferase or synthase as defined herein, having “reduced or abolished” activity means a decrease in its specific activity, i.e. reduced/abolished ability to catalyze formation of a product from a given substrate. A reduction by 100% is referred herein as abolishment of enzyme activity, achievable e.g. via deletion of the endogenous gene encoding said enzyme or blocking of the expression of said endogenous gene(s) with known methods.

Introduction of modification(s) in the host cell accumulating a lipophilic substance as defined herein in order to produce less or no copies of genes and/or proteins, such as transferases or synthases and respective genes as defined herein, including generation of modified suitable host cell capable of accumulation of fat-soluble vitamins, carotenoids or PUFAs as defined herein with reduced/abolished activity in enzymes corresponding to Yarrowia MNN9, optionally further comprising reduced/abolished activity in enzyme(s) corresponding to Yarrowia MNN10 and/or MNN11 and/or OCH1 and/or VAN1 and/or ANP1 and/or HOC1 and/or GAS1 and/or CHS4 may include the use of weak promoters, or the introduction of one or more mutation(s) (e.g. insertion, deletion/knocking-out or point mutation) of (parts of) the respective enzymes (as described herein), in particular its regulatory elements, leading to abolishment of said enzyme activity, such as e.g. inactivation via in vivo mutagenesis, for example by mutation of the catalytic residues or by making mutations or deletions that interfere with protein folding or pre- or pro-sequence cleavage such as e.g. needed to activate the transferase/synthase upon secretion by the host cell. The skilled person knows how to genetically manipulate or modify a host cell as defined herein resulting in reduction/abolishment of such activity, e.g. transferase or synthase activity, as defined herein. These genetic manipulations include, but are not limited to, e.g. gene replacement, gene amplification, gene disruption, transfection, transformation using plasmids, viruses, or other vectors. An example of such a genetic manipulation may for instance affect the interaction with DNA that is mediated by the N-terminal region of enzymes as defined herein or interaction with other effector molecules. In particular, modifications leading to reduced/abolished specific enzyme activity may be carried out in functional, such as functional for the catalytic activity, parts of the proteins. Furthermore, reduction/abolishment of enzyme specific activity might be achieved by contacting said enzymes with specific inhibitors or other substances that specifically interact with them.

The generation of a mutation into nucleic acids or amino acids, i.e. mutagenesis, may be performed in different ways, such as for instance by random or site-directed mutagenesis, physical damage caused by agents such as for instance radiation, chemical treatment, or insertion of a genetic element. The skilled person knows how to introduce mutations.

The terms “sequence identity”, “% identity” or “sequence homology” are used interchangeable herein. For the purpose of this invention, it is defined here that in order to determine the percentage of sequence homology or sequence identity of two amino acid sequences or of two nucleic acid sequences, the sequences are aligned for optimal comparison purposes. In order to optimize the alignment between the two sequences gaps may be introduced in any of the two sequences that are compared. Such alignment can be carried out over the full length of the sequences being compared. Alternatively, the alignment may be carried out over a shorter length, for example over about 20, about 50, about 100 or more nucleic acids/bases or amino acids. The sequence identity is the percentage of identical matches between the two sequences over the reported aligned region. The percent sequence identity between two amino acid sequences or between two nucleotide sequences may be determined using the Needleman and Wunsch algorithm for the alignment of two sequences (Needleman, S. B. and Wunsch, C. D. (1970) J. Mol. Biol. 48, 443-453). Both amino acid sequences and nucleotide sequences can be aligned by the algorithm. The Needleman-Wunsch algorithm has been implemented in the computer program NEEDLE. For the purpose of this invention the NEEDLE program from the EMBOSS package was used (version 2.8.0 or higher, EMBOSS: The European Molecular Biology Open Software Suite (2000) Rice, Longden and Bleasby, Trends in Genetics 16, (6) pp 276-277, http://emboss.bioinformatics.nl/). For protein sequences EBLOSUM62 is used for the substitution matrix. For nucleotide sequence, EDNAFULL is used. The optional parameters used are a gap-open penalty of 10 and a gap extension penalty of 0.5. The skilled person will appreciate that all these different parameters will yield slightly different results but that the overall percentage identity of two sequences is not significantly altered when using different algorithms.

After alignment by the program NEEDLE as described above the percentage of sequence identity between a query sequence and a sequence of the invention is calculated as follows: number of corresponding positions in the alignment showing an identical amino acid or identical nucleotide in both sequences divided by the total length of the alignment after subtraction of the total number of gaps in the alignment. The identity as defined herein can be obtained from NEEDLE by using the NOBRIEF option and is labeled in the output of the program as “longest identity”. If both amino acid sequences which are compared do not differ in any of their amino acids, they are identical or have 100% identity. With regards to enzymes originated from plants, the skilled person knows plant-derived enzymes might contain a chloroplast targeting signal which is to be cleaved via specific enzymes, such as e.g. chloroplast processing enzymes (CPEs).

Cultivation of the genetically modified host cells as defined herein and according to all embodiments of the present invention, particularly carotenoid-producing host cells, is performed as known in the art, such as cultivation of the appropriate non-modified host cell, such as cultivation in a suitable medium under suitable culture conditions. The lipophilic substances, preferably carotenoids, might be extracted from the host cell and subsequently purified, including chemical or physical separation methods such as extraction or chromatography.

Production of lipophilic substances such as e.g. fat soluble vitamins, carotenoids or PUFAs, preferably carotenoids, according to all embodiments of the present invention includes intracellular accumulation during the fermentation process followed by pre-treating the fermentation broth by mechanical, chemical and/or enzymatic means, followed by extraction or isolation of said substances from the host cell and the cultivation medium/fermentation broth.

The pre-treatment of the fermentation broth optionally includes pasteurization step at the end of fermentation.

The term “pre-treatment” according to the present invention comprises mechanical, chemical or enzymatic processes, leading to lysis of the cell wall to enable collecting the intracellular substances of interest. Such lysis processes particularly includes bead milling, enzyme treatment, and the like as known in the art, followed by extraction of the lipophilic products of interest with a suitable solvent.

Preferably, lysis by bead-milling as defined herein comprises about 1 to 5 passes, such as e.g. 1, 2, 3, 4, 5 whereby the amount of passes should be reduced as much as possible. More preferably, the pre-treatment of the cultivation broth does not contain any pass of bead-milling.

In a particular embodiment, the percentage of cell rupture measured via bead milling as pre-treatment method applied at the end of fermentation using a host cell as defined herein and according to all embodiments of the present invention, particularly wherein the host cell is a carotenoid-accumulating host cell, preferably accumulating AXN, said host cell furthermore comprising preferably a mutation in the MMN9 gene, particularly wherein said gene is inactivated, could be increased to about 80% as compared to a process using the respective wild-type host cell, i.e. respective carotenoid-accumulating host cell wherein the MNN9 endogenous gene is still expressed and active.

Pre-treatment as defined herein particularly includes one or more passes as defined herein of both mechanical and enzymatic lysis, optionally with washing steps between each pass, wherein the pre-treated broth including the lipophilic substances, i.e. free oil fraction, might be spray or freeze-dried and the lipophilic substances as defined herein furthermore extracted under suitable conditions using suitable solvents, particularly aqueous solvents.

As used herein, analysis of lipids, including lipophilic substances present in the free oil fraction obtained from the pre-treatment as defined herein, can be performed by methods known in the art, such as e.g. via gravimetric quantification, e.g. so-called FAME analysis, where the fatty acids are converted by transesterification to fatty acid methyl esters (FAME) and analyzed by gas chromatography (GC) as in WO2006052870.

Thus, in a very preferred embodiment, the present invention is directed to a genetically modified host cell, such as Yarrowia lipolytica, capable of accumulating a carotenoid, particularly AXN or ZEA, comprising acetylated forms such as mono- and di-acetylated forms, particularly with a percentage of 60-80% di-acetylated forms, comprising a modification, i.e. inactivation or disruption of endogenous MNN9 and optionally furthermore inactivation of endogenous CHS4 as well as to a process of production of carotenoids, such as e.g. AXN or ZEA, using said genetically modified host cell, comprising cultivating said cell under suitable culture conditions such that the carotenoids are intracellularly accumulated, pre-treating the fermentation broth at the end of fermentation with enzymes, particularly compositions comprising proteases, xylanases, alcalases, cellulases and mixtures thereof, up to 6 passes of bead milling, preferably wherein the passes of bead milling are reduced, more preferably with zero passes of bead milling, and extraction of the carotenoids, such as e.g. AXN or ZEA with a suitable solvent, particularly a solvent which is not dichloromethane, such as e.g. acetone, hexane, ethyl acetate, isobutyl acetate, octanol from the biomass and/or the cultivation medium.

The carotenoid as produced by such a process detailed above might be furthermore preferably selected from CXN, wherein only non-acetylated forms are produced and extracted as defined herein.

In one preferred embodiment, recovery of a lipophilic substance selected from carotenoids, particularly AXN, can be increased, wherein the AXN present in the free oil fraction is increased by at least 100%, such as e.g. 200, 400, 500, 600, 700 or even more, with using a suitable carotenoid-producing host cell, such as Yarrowia lipolytica producing AXN, and wherein said host cell comprises a mutation, preferably deletion, of endogenous MNN9, optionally combined with a mutation, preferably deletion, of endogenous CHS4.

Particularly, the present invention is directed to a process comprising:
(1) providing a host cell, particularly oleaginous yeast, capable of producing a lipophilic substance as defined herein;
(2) genetically modifying the host cell via introduction of a genetic modification in the mannan polymerase complexes subunit MNN9, particularly via reduction or abolishing the endogenous gene coding for mannan polymerase complexes subunit MNN9;
(3) cultivation of said genetically modified host cell under suitable conditions;
(4) harvesting and optionally pasteurization of the cultivation broth;
(5) treatment of the optionally pasteurized cultivation broth with enzymes, particularly proteases;
(6) solvent treatment or the lysed broth, particularly hexane treatment;
(7) concentration of the free oil phase comprising the accumulated lipophilic substance, particularly carotenoids, preferably AXN.

More particularly, such process according to the present invention comprising fermentation of a carotenoid-accumulating oleaginous yeast wherein one or more endogenous genes involved in mannan polymerase complex are genetically modified resulting in less enzyme activity, preferably comprising deletion of MNN9, leads to increase in the recovery of free oil comprising the target product of interest, such as preferably AXN, wherein through the deletion of MNN9 the accumulation of the carotenoids, particularly AXN, could be increased from 1% to at least about 8%, wherein the “increase in the recovery of free oil” means the fraction present as free oil after enzymatic and/or mechanical lysis of the respective host cells as defined herein. Even more preferred is a process wherein the accumulated AXN is in the form of mono- and/or de-acetylated AXN.

In a particular preferred embodiment, the present invention includes production of AXN comprising di- and mono-acetylated forms, wherein the percentage of acetylated-AXN is increased from 34 to 54%.

### FIGURES

FIG. 1. Experimental conditions for Downstream Processing (DSP) of the Yarrowia broth comprising the accumulated lipophilic substance including pasteurization, enzyme lysis, washing and analysis of the biomass comprising free oil and biomeal.

The following examples are illustrative only and are not intended to limit the scope of the invention in any way. The contents of all references, patent applications, patents, and published patent applications, cited throughout this application are hereby incorporated by reference, in particular U.S. Pat. No. 9,297,031, WO2016172282, WO2008073367, WO2008130372, WO2003097798, WO2014096990, WO2006102342, WO2014096992, WO2006052870, WO2006052871.

### EXAMPLES

### Example 1: General Methods, Strains and Sequences

All basic molecular biology and DNA manipulation procedures described herein are generally performed according to Sambrook et al. (eds.), Molecular Cloning: A Laboratory Manual. Cold Spring Harbor Laboratory Press: New York (1989) or Ausubel et al. (eds). Current Protocols in Molecular Biology. Wiley: New York (1998).

DNA transformation. As described in WO2014096992.

DNA molecular biology. CRISPR Cas9 was used to target the respective gene, e.g. MNN9 gene, and it generated an insertion, such as e.g. a 2 bp insertion early in the mnn9 gene, thereby introducing a frame shift and inactivating the gene product. To introduce the mutation the strain was transformed with an epigenic plasmid with a hygromycin resistance marker expressing the Cas9 targeting the 5′ region of the gene of interest, in this case MNN9 plasmid MB7549. Isolates resistant 100 ug/ml of hygromycin were pooled and diluted and single colonies were sequenced by amplifying the gene with primers 200 bp upstream and downstream of the gene and sanger sequencing using the upstream primer. Thirty percent of the secondary isolates had a missense or frameshift mutation, and the frame shift mutations were isolated and screened for activity. Introduction of CAS9 chs4 (plasmid MB7547), or CAS9 gas1 (plasmid MB7550) single mutations were performed accordingly. For the double mutation the mutant strain was cured of the plasmid by serial passage and monitoring the loss of the hygromycin gene, then the second plasmid (MB7547 cas9 chs4) was introduced and screened as described above.

Construction of the AXN-accumulating host cell in which the above-mentioned gene deletions are introduced is described in WO2014096992.

Bead milling: 1 L of broth was milled by four passes through the bead mill with samples taken before and after each pass. Results were monitored by hemocytometer (direct cell counts under the microscopic) and visual assessment of viscosity (increased viscosity of samples correlates with increased lysis). Enhanced cell rupture observed by hemocytometer and/or by viscosity indicates that a given strain is more susceptible to physical lysis by bead mill. To monitor lysis by extractability cells are subjected to extraction by excess solvent the untreated cells accounts for 0% lysis and a positive control made by lysing the cell with a Precellys® disruption system (Bertin Instruments, Rockville, MD) demonstrated 100% lysis.

Enzymatic cell lysis: fermentation broth was pretreated with a broad specificity protease (Alcalase®, Novozymes) and a R-glucanase preparation (DSM Filtrase® NL, DSM). Alcalase treatment was done with 3.3, 6.6, 10 g/kg biomass for 24 hours. Lysis was monitored as mentioned above.

Plasmid list. Plasmid, strains, nucleotide and amino acid sequences that were used are listed in Table 1, 2 and the sequence listing. In general, all non-modified sequences referred to herein are the same as the accession sequence in the database for reference strain CLIB122 (Dujon B, et at, Nature. 2004 Jul. 1; 430(6995):35-44).

TABLE 1
sequences and plasmids used to generate the knock-out mutants.

| | SEQ | |
|---|---|---|
| | ID | |
| Name | NO: | 5′ to 3′ sequence |
| MNN9 | 1 | MRSARRYLLVLLPVVALLYVLSTYFRGTPVEKPVARNMAGNHIVKYNMNKLQASSQ |
| | | AASHSEKVLILTPLARFYDEYWENLVALSYPHDLIELGFIVPRSKEGQQALEKLDR |
| | | AVAKTQKGKDKFSKVTILROEAEDALASHSEKDRHAMSVQKARRSAMSLARNSLFL |
| | | STIGPDTAWILWLDSDVVETPHTLIQDMTKHDKDVLVANCYQRYTNDQGKPDVRPY |
| | | DYNSWVESQQGIKMAESMSPDDIILEGYGEMATYRLLMAKIYNADGDIHEEIALDG |
| | | VGGTALLVKSEVHRDGAMFPPFPFYNLIETEGFAKMAKRLGYKPYGLPNYLVYHYN |
| | | E |
| MNN9 | 2 | ATGCGTTCGGCTCGACGGTATCTGCTTGTGCTTCTACCGGTAGTCGCTCTGCTGTA |
| | | TGTGCTGAGCACCTACTTCCGCGGCACGCCCGTTGAAAAACCCGTGGCTCGAAACA |
| | | TGGCCGGAAATCACATTGTCAAGTACAATATGAACAAGCTGCAGGCGTCGTCGCAG |
| | | GCGGCTAGCCACAGCGAGAAGGTGCTGATTCTCACTCCTCTGGCTCGTTTCTACGA |
| | | CGAGTACTGGGAGAACCTTGTGGCTCTGAGCTACCCTCACGACCTCATCGAGCTGG |
| | | GCTTCATTGTTCCTCGAAGCAAGGAGGGCCAACAGGCGCTGGAGAAGCTCGATCGA |
| | | GCTGTCGCGAAGACCCAGAAGGGCAAGGACAAGTTTTCTAAGGTGACCATTCTGCG |
| | | ACAGGAGGCTGAGGACGCTTTGGCTTCACATTCGGAAAAGGACCGACACGCGATGT |
| | | CTGTGCAGAAGGCCCGGCGAAGTGCCATGTCTCTGGCTCGAAACTCACTGTTCCTG |
| | | TCCACCATTGGCCCCGACACTGCATGGATTCTGTGGCTGGACTCGGACGTGGTGGA |
| | | GACCCCCCATACCCTCATCCAGGACATGACCAAACATGACAAGGACGTTCTGGTGG |
| | | CTAATTGCTATCAGCGATACACCAACGACCAGGGCAAGCCCGACGTGCGGCCCTAC |
| | | GACTACAACTCGTGGGTCGAGTCGCAACAGGGCATCAAGATGGCCGAGTCCATGTC |
| | | CCCTGACGACATCATTCTCGAGGGCTACGGAGAAATGGCCACCTACCGACTGCTCA |
| | | TGGCCAAGATCTACAACGCCGATGGAGACATTCACGAGGAAATTGCCCTCGACGGT |
| | | GTCGGAGGAACCGCTCTGCTGGTTAAGTCCGAGGTGCACCGTGATGGAGCCATGTT |
| | | TCCTCCTTTCCCCTTTTACAACCTCATCGAGACAGAGGGTTTTGCCAAGATGGCCA |
| | | AGCGACTTGGCTACAAGCCTTACGGTCTTCCCAACTATCTCGTCTACCATTACAAT |
| | | GAGTAG |
| CHS4 | 3 | MDSYNKFGFNSETGEPNTGPQDTSGSVPSEHRGDGSFSPDIGRKKSLIRPDRARLD |
| | | PDSRTYHYSQLAAQQQSRINVLPSSTGNDPILEEHYMRETGEIQPGQGYDQPYDNA |
| | | VNQEVLEEENEESYREGEEDPYAYSGRQVYGLNDEVQTEAYVRKSDEISSSEEQAA |
| | | DTQPPIIDPPVNEKVQLSRGGGTLKRHNSVKRRMGTQPKQISLWMTYCQIVTFWAP |
| | | APVLKMFGMPQKARQDAWREKIGLITVILYIAAFVAYLTFGFTITVCSSSITRVRN |
| | | GEVNSGNLIINGKQYDFTSMNHPPTVEVPTGGNFMYPPVNAGSMDGSLLFQNVNNH |
| | | CKGLITPKPDCKIPYNGDSVAWYMPCQLLNQDGSSQPNTTEQFYPGYACHTSTTAR |
| | | NVYYHQLVDAGEVYFTWDDIKNSTRNLAVFNGAVIDLNMVNFILKDDLNYPEIFDT |
| | | LRDDDSIKGTDISRNMANGPDRQAARCLQEIAKVGYIDTEQIGCLASQVVLYVSLV |
| | | FIISIVAVKFLFAIYYKWAISAKQGAFPIDNKQMNQRENEIEDWSENIYSQAPIKR |
| | | VDRDQPKSSATPFRGLIKHRFNQHDNKNKRMSTMVTQVGESGTAPSVYSRNSAAID |
| | | SNTDVGGVVYDYDSGSQPYEAPTVNVVPQPPIDYQPFNYPLAHTMCLVTAYSESID |
| | | GLRTTLDSITTTDYPNSHKLIVIIADGIIKGSGNDMSTPDICLSMMCDMPIPEDQV |
| | | RPFSYVSTSSGTKRHNMAKVYSGFYAYDDATVPPEKQQRVPVVTIVKCGTPAEANE |
| | | AKPGNRGKRDSQIILMSFLQRVMFDERMTELEHEMFNGIWRVTGILPDFYEIILMV |
| | | DADTKVFPDSLTHMVAEMVKDPEIMGLCGETKIANKRDSWVTAIQVFEYYISHHQA |
| | | KAFESVFGGVTCLPGCFSMYRIKAPKGGDGYWVPILANPDIVERYADNVIDTLHKK |
| | | NLLLLGEDRYLTSLMLKTFPTRKQVFVSKAVCKTIVPDKFSVLLSQRRRWINSTVH |
| | | NLMELVLVKDLCGVFCISMQFVVFIELVGTLILPAAISFTIYVVIIAIVRKPTPVM |
| | | SLVLLALILGLPGLLIIITASRWSYVVWMFIYLLSLPIWNFVLPVYAYWKFDDFSW |
| | | GDTRQVAGEKKGSGGHDEEDGEFDSSQIVMKRWRDFEMDKRRYVPLPPQNAWMSDP |
| | | HMPSYNHQGFSANSSPALRPQGSSHVYSDSVAGASDYNVAQGHEVL |
| CHS4 | 4 | ATGGATTCGTACAACAAATTCGGATTCAACTCGGAGACGGGTGAGCCCAACACTGG |
| | | CCCCCAGGACACATCTGGCAGTGTCCCCAGTGAACACAGAGGCGACGGCTCGTTCT |
| | | CCCCCGACATTGGACGAAAGAAGTCTCTCATTCGACCTGACCGAGCTCGACTCGAC |
| | | CCCGACTCGCGAACTTATCACTACTCGCAGCTGGCCGCCCAGCAGCAAAGCAGAAT |
| | | CAACGTGCTTCCCAGTTCCACCGGAAATGACCCTATTCTCGAGGAGCACTACATGC |
| | | GCGAGACTGGCGAGATCCAGCCTGGTCAGGGATACGATCAGCCCTACGACAACGCC |
| | | GTGAACCAGGAGGTGCTAGAGGAGGAAAACGAGGAATCATACAGAGAGGGAGAGGA |
| | | GGACCCTTACGCCTACTCGGGCCGTCAGGTCTATGGTCTCAATGACGAGGTCCAAA |
| | | CCGAGGCCTACGTGCGAAAGTCGGACGAAATCTCCTCATCAGAAGAACAGGCTGCC |
| | | GACACCCAACCGCCCATCATCGACCCACCTGTCAACGAAAAGGTTCAGCTCAGCCG |
| | | TGGAGGAGGAACACTGAAGCGACACAACTCCGTCAAGCGACGAATGGGCACCCAAC |
| | | CCAAGCAGATTTCGCTGTGGATGACCTACTGTCAGATTGTTACCTTCTGGGCCCCC |
| | | GCTCCTGTGCTCAAGATGTTTGGCATGCCCCAAAAAGCCCGACAGGACGCCTGGCG |
| | | AGAAAAGATTGGCCTCATCACTGTTATTCTTTACATTGCCGCCTTTGTTGCATACC |
| | | TGACTTTCGGCTTCACCATCACAGTTTGTTCCAGTTCCATCACCCGAGTGCGAAAC |
| | | GGAGAAGTCAACTCTGGAAACCTCATCATTAATGGTAAGCAATACGACTTTACCTC |
| | | TATGAACCACCCTCCCACAGTCGAGGTTCCTACTGGCGGTAACTTCATGTACCCTC |
| | | CTGTTAATGCTGGATCCATGGACGGTTCTTTGCTGTTCCAGAACGTCAACAACCAC |
| | | TGCAAGGGTCTAATCACCCCCAAGCCCGACTGTAAAATTCCCTACAACGGCGACAG |
| | | CGTTGCCTGGTATATGCCCTGCCAGCTGCTCAACCAGGACGGATCTTCCCAACCCA |
| | | ACACTACTGAGCAGTTCTACCCCGGCTACGCATGCCACACTTCCACCACCGCTCGA |
| | | AACGTCTACTACCACCAGCTGGTTGATGCCGGTGAGGTCTACTTCACCTGGGACGA |
| | | TATCAAGAACTCTACTCGAAACCTGGCTGTGTTCAACGGAGCTGTCATTGACCTGA |
| | | ACATGGTCAACTTCATTCTTAAGGACGACCTCAACTACCCCGAGATCTTTGACACT |
| | | CTGCGAGATGACGATTCCATCAAGGGTACTGATATCTCTCGTAATATGGCCAACGG |
| | | TCCTGATCGACAGGCTGCCCGATGTCTGCAAGAGATTGCCAAGGTTGGATACATTG |
| | | ATACCGAGCAGATTGGATGTCTGGCTTCCCAGGTTGTTCTGTATGTATCTCTGGTT |
| | | TTCATTATCTCCATTGTCGCCGTCAAGTTCCTATTTGCTATCTACTACAAGTGGGC |
| | | CATTTCTGCCAAGCAGGGAGCTTTCCCCATTGACAACAAGCAGATGAACCAGCGAG |
| | | AGAACGAGATTGAGGACTGGTCCGAGAACATTTACAGCCAGGCTCCCATCAAGCGA |
| | | GTGGACCGAGACCAGCCTAAGTCCAGTGCCACTCCCTTCCGTGGCCTCATCAAGCA |
| | | CCGATTCAACCAGCACGACAATAAGAACAAGCGAATGAGCACAATGGTGACCCAGG |
| | | TTGGTGAGAGTGGAACTGCTCCTTCCGTCTACTCTCGAAACTCCGCTGCGATCGAC |
| | | TCCAACACTGATGTTGGCGGTGTTGTCTACGATTACGACTCTGGATCTCAGCCCTA |
| | | CGAGGCGCCCACCGTCAACGTTGTGCCCCAGCCCCCCATCGACTACCAACCGTTCA |
| | | ACTACCCTCTCGCCCACACCATGTGTCTGGTGACTGCATACTCCGAGTCGATTGAC |
| | | GGTCTGAGAACCACCCTTGACTCTATCACCACAACCGACTACCCTAACTCCCATAA |
| | | ACTCATTGTTATCATTGCCGATGGTATTATTAAGGGTTCCGGTAACGACATGTCTA |
| | | CCCCTGACATTTGTCTAAGTATGATGTGTGATATGCCTATCCCCGAAGACCAGGTG |
| | | AGACCCTTCTCTTACGTATCTACTTCTTCTGGTACAAAGCGTCATAACATGGCCAA |
| | | GGTCTACTCTGGTTTCTACGCCTACGATGATGCCACAGTTCCACCTGAGAAGCAGC |
| | | AGCGAGTCCCCGTTGTGACTATTGTCAAGTGCGGTACGCCTGCTGAGGCCAACGAG |
| | | GCCAAGCCTGGTAACCGAGGTAAGCGAGACTCCCAGATCATTCTCATGTCGTTCTT |
| | | GCAGCGTGTCATGTTCGACGAGCGAATGACCGAGCTGGAGCACGAGATGTTCAACG |
| | | GTATCTGGCGAGTGACTGGTATTCTTCCCGACTTTTATGAAATCATTCTCATGGTG |
| | | GATGCCGATACTAAGGTGTTCCCTGACTCTCTGACCCACATGGTGGCTGAGATGGT |
| | | CAAGGATCCCGAGATTATGGGACTATGTGGAGAAACCAAGATTGCCAACAAGCGAG |
| | | ACTCTTGGGTCACTGCGATCCAGGTGTTTGAATACTACATTTCTCATCACCAGGCT |
| | | AAGGCCTTTGAGTCCGTCTTTGGTGGTGTCACTTGTCTGCCTGGTTGTTTCTCCAT |
| | | GTACCGAATCAAGGCTCCCAAGGGCGGAGACGGCTACTGGGTGCCCATTCTTGCCA |
| | | ACCCTGATATCGTCGAGCGATACGCTGACAACGTGATTGACACTCTGCATAAGAAG |
| | | AACCTGTTGCTTCTCGGAGAAGATCGATACCTGACCTCCCTCATGCTCAAGACCTT |
| | | CCCCACGCGAAAACAGGTGTTTGTGTCCAAGGCTGTCTGCAAGACCATTGTTCCCG |
| | | ACAAGTTTTCTGTTTTGCTGTCTCAGCGAAGACGATGGATTAACTCCACTGTGCAT |
| | | AACCTGATGGAGCTGGTGCTGGTCAAGGATCTATGTGGTGTCTTCTGTATTTCCAT |
| | | GCAGTTTGTTGTCTTCATCGAGCTTGTTGGTACCCTGATTCTTCCCGCCGCCATCT |
| | | CGTTCACCATCTACGTTGTCATTATTGCCATTGTGAGAAAACCTACGCCTGTCATG |
| | | TCGCTGGTTCTGCTGGCTCTGATTTTGGGTCTGCCGGGTCTTCTCATTATCATCAC |
| | | AGCATCTCGATGGTCATACGTCGTGTGGATGTTCATCTACCTGCTATCTCTTCCCA |
| | | TTTGGAACTTTGTGCTCCCCGTCTACGCATACTGGAAGTTTGATGATTTCTCCTGG |
| | | GGTGACACTCGACAGGTTGCTGGTGAAAAGAAGGGCTCTGGAGGCCACGACGAGGA |
| | | GGATGGTGAATTTGACTCGTCTCAGATTGTCATGAAGCGATGGAGAGACTTTGAAA |
| | | TGGACAAGCGACGCTACGTGCCACTGCCTCCCCAGAACGCGTGGATGAGCGATCCT |
| | | CACATGCCTTCTTACAACCACCAAGGATTTTCTGCTAACTCTTCCCCTGCTCTTAG |
| | | ACCCCAGGGCTCTTCTCATGTCTACTCGGACTCGGTTGCCGGAGCCAGTGACTACA |
| | | ACGTTGCTCAAGGCCACGAAGTACTTTAA |
| GAS1 | 5 | MKFSAVSIAAALASLVAAADVPSIDIVGNKFFYSNNGSQFYIRGVAYQQDSANETA |
| | | KETFSDPLSDGDACKRDLPYLVQLKTNVLRVYAIDTTKDHDTCMQLFNDAGIYVIS |
| | | DLSQPGESINRNSPSWDVDLYQRYTSVVDTMQKYPNMLGFFAGNEVTNNNTNTDAS |
| | | PYVKAAIRDMKSYMKEKSYRSIPVGYATNDDEKTRDDLATYFQCGNASEAADFYGI |
| | | NIYEWCGDATFESSGYQQRTEEYKNFTIPIFFSEYGCNRVKPRQFSDVPVLFGPNM |
| | | TDVWSGGIVYMYFEEQNQYGLVSVSGSSVSTLDDFNNLSSQMAKISPSSANTNDYK |
| | | PSQTAGGVSCPDFNANWKGSPTLPPTPKVGTCQCINDGSSCVVNSDTNAKNYGDLF |
| | | GELCGNLDVDCSEIQSNGTSGKYGDLSFCDAKQKLNFLMGKYYDSONGNSQACDFS |
| | | SQASIVQSPKTGSNCQNAQATAVSSGSSGSSGSSGSGSSGSGSSGSSGSGSGSAKG |
| | | SAAAAASGSAAASSSKGGNKKSAGASLAAEKVVASVVFGAIAIMAVIV |
| GAS1 | 6 | ATGAAGTTCTCAGCGGTCTCAATCGCTGCTGCCCTGGCCTCGCTGGTGGCAGCAGC |
| | | AGACGTGCCCTCTATTGACATTGTGGGCAACAAGTTCTTCTACTCCAACAACGGCT |
| | | CTCAATTCTACATCCGAGGAGTGGCCTACCAGCAGGACTCGGCCAACGAGACAGCC |
| | | AAGGAGACCTTTTCGGACCCTCTGTCGGACGGCGACGCCTGTAAGCGAGACCTGCC |
| | | CTACCTGGTGCAGCTCAAGACCAACGTGCTTCGAGTGTACGCCATCGACACCACCA |
| | | AGGACCACGACACATGCATGCAGCTCTTCAACGACGCCGGCATCTACGTCATTTCC |
| | | GATCTCTCACAGCCCGGCGAGTCCATCAACCGAAACTCGCCCTCGTGGGACGTGGA |
| | | TCTGTACCAGCGATACACCAGCGTAGTGGACACCATGCAAAAGTACCCCAACATGC |
| | | TGGGTTTCTTTGCCGGAAACGAGGTCACTAACAACAACACCAACACCGATGCGTCT |
| | | CCCTACGTCAAGGCTGCCATCCGAGACATGAAGTCGTACATGAAGGAGAAGTCGTA |
| | | CCGATCCATTCCCGTCGGATACGCCACCAACGATGACGAGAAGACCCGAGACGACC |
| | | TGGCCACCTATTTCCAGTGCGGCAACGCCTCCGAGGCAGCCGATTTCTACGGAATC |
| | | AACATCTACGAGTGGTGTGGAGACGCCACCTTTGAGTCCTCCGGTTACCAGCAGCG |
| | | AACCGAGGAGTACAAGAACTTCACCATCCCCATCTTCTTCTCCGAGTACGGCTGCA |
| | | ACCGAGTCAAGCCGCGACAGTTCTCCGACGTGCCCGTTCTGTTTGGCCCCAACATG |
| | | ACCGACGTCTGGTCCGGAGGAATCGTCTACATGTACTTTGAGGAGCAGAACCAGTA |
| | | CGGTCTGGTGTCTGTTTCCGGAAGCTCCGTCTCCACTCTCGACGACTTCAACAACC |
| | | TGTCTTCGCAGATGGCTAAGATTTCTCCTTCCTCCGCCAACACCAACGACTACAAG |
| | | CCCTCTCAGACCGCTGGTGGCGTCTCGTGCCCCGACTTCAACGCCAACTGGAAAGG |
| | | CTCTCCCACCCTGCCTCCCACCCCCAAGGTCGGAACCTGTCAGTGCATCAACGATG |
| | | GCTCTTCTTGTGTCGTCAACTCGGACACTAACGCCAAGAACTACGGCGATCTCTTT |
| | | GGAGAGCTGTGCGGTAACCTCGATGTGGACTGCTCCGAGATCCAGTCCAACGGAAC |
| | | CTCCGGCAAGTACGGAGACCTGTCCTTCTGTGACGCCAAGCAGAAGCTCAACTTCC |
| | | TCATGGGCAAGTACTACGACTCCCAGAACGGCAACTCTCAGGCCTGCGACTTTTCT |
| | | TCCCAGGCCTCCATTGTCCAGAGCCCCAAGACCGGATCCAACTGCCAGAACGCCCA |
| | | GGCCACTGCTGTCTCTTCCGGCTCGTCCGGCTCTTCCGGCTCGTCCGGCTCTGGAT |
| | | CTTCGGGCTCTGGATCGTCCGGATCCTCGGGCTCTGGCTCGGGCTCTGCTAAGGGC |
| | | TCTGCTGCTGCCGCTGCCTCTGGCTCGGCCGCCGCCTCGTCCTCCAAGGGCGGAAA |
| | | CAAGAAGAGCGCCGGCGCCTCGCTGGCTGCCGAGAAGGTGGTTGCCTCCGTCGTGT |
| | | TTGGCGCCATCGCCATCATGGCCGTTATTGTTTAA |
| MB7547 | 7 | GGTTCCGCTTCCTCGCTCACTGACTCGCTGCGCTCGGTCGTTCGGCTGCGGCGAGC |
| | | GGTATCAGCTCACTCAAAGGCGGTAATACGGTTATCCACAGAATCAGGGGATAACG |
| | | CAGGAAAGAACATGTGAGCAAAAGGCCAGCAAAAGGCCAGGAACCGTAAAAAGGCC |
| | | GCGTTGCTGGCGTTTTTCCATAGGCTCCGCCCCCCTGACGAGCATCACAAAAATCG |
| | | ACGCTCAAGTCAGAGGTGGCGAAACCCGACAGGACTATAAAGATACCAGGCGTTTC |
| | | CCCCTGGAAGCTCCCTCGTGCGCTCTCCTGTTCCGACCCTGCCGCTTACCGGATAC |
| | | CTGTCCGCCTTTCTCCCTTCGGGAAGCGTGGCGCTTTCTCATAGCTCACGCTGTAG |
| | | GTATCTCAGTTCGGTGTAGGTCGTTCGCTCCAAGCTGGGCTGTGTGCACGAACCCC |
| | | CCGTTCAGCCCGACCGCTGCGCCTTATCCGGTAACTATCGTCTTGAGTCCAACCCG |
| | | GTAAGACACGACTTATCGCCACTGGCAGCAGCCACTGGTAACAGGATTAGCAGAGC |
| | | GAGGTATGTAGGCGGTGCTACAGAGTTCTTGAAGTGGTGGCCTAACTACGGCTACA |
| | | CTAGAAGGACAGTATTTGGTATCTGCGCTCTGCTGAAGCCAGTTACCTTCGGAAAA |
| | | AGAGTTGGTAGCTCTTGATCCGGCAAACAAACCACCGCTGGTAGCGGTGGTTTTTT |
| | | TGTTTGCAAGCAGCAGATTACGCGCAGAAAAAAAGGATCTCAAGAAGATCCTTTGA |
| | | TCTTTTCTACGGGGTCTGACGCTCAGTGGAACGAAAACTCACGTTAAGGGATTTTG |
| | | GTCATGAGATTATCAAAAAGGATCTTCACCTAGATCCTTTTAAATTAAAAATGAAG |
| | | TTTTAAATCAATCTAAAGTATATATGAGTAAACTTGGTCTGACAGTTACCAATGCT |
| | | TAATCAGTGAGGCACCTATCTCAGCGATCTGTCTATTTCGTTCATCCATAGTTGCC |
| | | TGACTCCCCGTCGTGTAGATAACTACGATACGGGAGGGCTTACCATCTGGCCCCAG |
| | | TGCTGCAATGATACCGCGAGACCCACGCTCACCGGCTCCAGATTTATCAGCAATAA |
| | | ACCAGCCAGCCGGAAGGGCCGAGCGCAGAAGTGGTCCTGCAACTTTATCCGCCTCC |
| | | ATCCAGTCTATTAATTGTTGCCGGGAAGCTAGAGTAAGTAGTTCGCCAGTTAATAG |
| | | TTTGCGCAACGTTGTTGCCATTGCTGCAGGCATCGTGGTGTCACGCTCGTCGTTTG |
| | | GTATGGCTTCATTCAGCTCCGGTTCCCAACGATCAAGGCGAGTTACATGATCCCCC |
| | | ATGTTGTGCAAAAAAGCGGTTAGCTCCTTCGGTCCTCCGATCGTTGTCAGAAGTAA |
| | | GTTGGCCGCAGTGTTATCACTCATGGTTATGGCAGCACTGCATAATTCTCTTACTG |
| | | TCATGCCATCCGTAAGATGCTTTTCTGTGACTGGTGAGTACTCAACCAAGTCATTC |
| | | TGAGAATAGTGTATGCGGCGACCGAGTTGCTCTTGCCCGGCGTCAACACGGGATAA |
| | | TACCGCGCCACATAGCAGAACTTTAAAAGTGCTCATCATTGGAAAACGTTCTTCGG |
| | | GGCGAAAACTCTCAAGGATCTTACCGCTGTTGAGATCCAGTTCGATGTAACCCACT |
| | | CGTGCACCCAACTGATCTTCAGCATCTTTTACTTTCACCAGCGTTTCTGGGTGAGC |
| | | AAAAACAGGAAGGCAAAATGCCGCAAAAAAGGGAATAAGGGCGACACGGAAATGTT |
| | | GAATACTCATACTCTTCCTTTTTCAATATTATTGAAGCATTTATCAGGGTTATTGT |
| | | CTCATGAGCGGATACATATTTGAATGTATTTAGAAAAATAAACAAATAGGGGTTCC |
| | | GCGCACATTTCCCCGAAAAGTGCCACCTGACGTCTAAGAAACCATTATTATCATGA |
| | | CATTAACCTATAAAAATAGGCGTATCACGAGGCCCTTTCGTCTGGCCTAGGAAGCG |
| | | ACTTCCAATCGCTTTGCATATCCAGTACCACACCCACAGGCGTTTGTGCTACTCTA |
| | | CTGATAGCAATAGATGCGTCATAATTGGTTGGCCCGCTGAGCCTCCACAGGATACT |
| | | ATTGCACATACCCTGGTCATGTGCAGATCAGCTCATTTGTGGAGACTCTGGAGTAA |
| | | CTTAGACGACGCCTGGTTCAATTGCCGCAATGTGCGCCCACGCAGATAATGTATTG |
| | | AGGGGTGGAGCGCCTCTTGGGGACTTGCTGTACTTGTACGGGATATTAAACGCACT |
| | | CAGCAAGACCATGACGTAAAACACACCTACTGTACGATACGTACTGTAGGTATTGT |
| | | ACTCGTACCCGGTACTACAAATAGTACGATACTATACGGAGTGTATTTGTACCTTG |
| | | ATATACGACTGGCGGAGTGAAGAGAAGGAGTTGAACAAGACCAGATGGGGATATCA |
| | | GCCCCAGTGCTTTGTATTACAAGTACGAGTACTTAATAGATACTGTAAGGCTATTG |
| | | ATACGGATGGCAGTAAGTCATTGAGTAAGCAATTGTGGCCCAGCATCTCCCCTACG |
| | | TACTTGTACCATACCCCATGGAGACACCAATGGTCTTTCACGCACACTGTCGTGTG |
| | | CTGTATCGCAGAATCGGGTGTCCAACCAAATGCCGTTACCCCCACGTCACAGCCGA |
| | | TAGACAGATACACCATCAATACCAGCAGGTTGTATCATGCGGTTGGCTGAAGGTAA |
| | | GCTGATTGGTCTAAAAACTGTAGCTGTCCTAATTCAACGAGCGCTATTTGGGGCCA |
| | | ACCACCTCGGCCAAGCGGCCTTTAATCTGCGTGCCCCAGAGGCGTCTAATGAGGCT |
| | | CTGGCCGCCACTGTAGGAGTGTTTCTCTGTGCGCACACGCAGTTTTGAGTTTGGGC |
| | | GACTTTCCCTTTTTCCCAATTGCGTACACACACAGCTCCGAGCTAAGCGCTGTCCT |
| | | TGAACCTTCTCCCTCTTTTCCCTCTTTTTCTCTTCCCCTTCCCCTCCTCCACATTA |
| | | AGGCCAAATCCTGAATTGCACCAACTAGTACAACGACAACAATGGACAAGAAGTAC |
| | | TCCATCGGTTTGGACATTGGTACTAACTCTGTCGGCTGGGCCGTCATCACCGACGA |
| | | GTACAAGGTTCCCTCCAAGAAGTTCAAGGTCCTTGGCAACACCGACCGACACTCTA |
| | | TCAAGAAGAACCTGATCGGTGCTCTGCTGTTCGACTCTGGCGAGACTGCCGAGGCC |
| | | ACCCGACTGAAGCGAACCGCTCGACGCCGATACACCCGACGAAAGAACCGAATCTG |
| | | TTACCTCCAGGAGATCTTCAGCAACGAGATGGCTAAGGTCGACGACTCCTTCTTCC |
| | | ACCGACTCGAGGAGTCTTTCCTGGTCGAAGAGGATAAGAAGCACGAGCGACACCCC |
| | | ATCTTCGGCAACATTGTTGATGAGGTTGCCTACCATGAGAAGTACCCCACCATCTA |
| | | CCACCTCCGAAAGAAGCTCGTCGACTCCACTGACAAGGCTGACCTCCGACTCATCT |
| | | ACCTTGCTCTCGCCCACATGATCAAGTTCCGAGGTCACTTCCTCATTGAGGGTGAT |
| | | CTCAACCCCGACAACTCCGACGTTGACAAGCTGTTCATCCAGCTCGTCCAGACCTA |
| | | CAACCAGCTCTTTGAGGAGAACCCTATCAACGCTTCTGGTGTTGACGCCAAGGCCA |
| | | TTCTCTCCGCCCGACTCTCTAAGTCCCGACGACTCGAGAACCTCATTGCCCAGCTG |
| | | CCCGGCGAGAAGAAGAACGGCCTCTTCGGTAACCTGATTGCTCTCTCTCTTGGTCT |
| | | GACCCCCAACTTCAAGTCCAACTTTGACCTCGCCGAGGACGCCAAGCTCCAGCTGT |
| | | CCAAGGACACCTACGATGACGATCTGGACAACCTCCTGGCCCAGATCGGTGACCAG |
| | | TACGCCGATCTCTTCCTTGCCGCCAAGAACCTCTCCGACGCCATCCTGCTCTCCGA |
| | | CATCCTCCGAGTCAACACCGAGATTACCAAGGCTCCTCTGTCTGCCTCTATGATCA |
| | | AGCGATACGACGAGCACCACCAGGATCTCACTCTTCTCAAGGCTCTCGTCCGACAG |
| | | CAGCTCCCCGAGAAGTACAAGGAGATTTTCTTTGACCAGTCCAAGAACGGTTACGC |
| | | TGGCTACATTGACGGTGGTGCTTCCCAGGAAGAGTTTTACAAGTTCATCAAGCCTA |
| | | TTCTGGAGAAGATGGACGGTACCGAGGAGCTGCTCGTCAAGCTCAACCGAGAGGAC |
| | | CTCCTTCGAAAGCAGCGAACCTTCGATAACGGCTCCATCCCCCACCAGATCCACCT |
| | | GGGTGAGCTCCACGCCATTCTCCGAAGACAAGAGGACTTCTACCCCTTCCTAAAGG |
| | | ATAACCGAGAGAAGATCGAGAAGATTCTCACCTTCCGAATCCCCTACTACGTCGGT |
| | | CCCCTCGCTCGAGGTAACTCCCGATTTGCTTGGATGACCCGAAAGTCCGAGGAGAC |
| | | TATCACCCCCTGGAACTTTGAAGAGGTAGTCGACAAGGGTGCCTCCGCCCAGTCTT |
| | | TCATTGAGCGGATGACCAACTTCGATAAGAACCTCCCCAACGAGAAGGTCCTTCCC |
| | | AAGCACTCTCTCCTCTACGAGTACTTCACCGTCTACAACGAGCTGACCAAGGTCAA |
| | | GTACGTTACCGAGGGCATGCGAAAGCCCGCTTTCCTCTCTGGTGAGCAGAAGAAGG |
| | | CCATTGTCGACCTCCTGTTCAAGACTAACCGAAAAGTCACCGTCAAGCAGCTCAAG |
| | | GAAGACTACTTCAAGAAGATTGAGTGCTTCGACTCCGTCGAGATTTCCGGTGTCGA |
| | | GGACCGATTCAACGCCTCCCTCGGCACCTACCACGATCTTCTGAAGATCATCAAGG |
| | | ACAAGGACTTTCTTGATAACGAGGAGAACGAGGACATTCTCGAGGACATCGTCCTC |
| | | ACCCTCACCCTTTTCGAGGATCGAGAGATGATCGAGGAGCGACTCAAGACCTACGC |
| | | CCATCTCTTCGACGACAAGGTCATGAAGCAACTCAAGCGACGACGATACACTGGCT |
| | | GGGGCCGACTTTCCCGAAAGCTCATCAACGGCATCCGAGACAAGCAGTCTGGCAAG |
| | | ACCATCCTGGACTTCCTGAAGTCCGACGGTTTCGCCAACCGAAACTTCATGCAGCT |
| | | CATCCACGACGACTCTCTTACCTTCAAAGAGGATATCCAGAAGGCCCAGGTTTCTG |
| | | GCCAGGGCGACTCCCTCCACGAGCACATTGCCAACCTCGCCGGATCCCCCGCCATC |
| | | AAAAAGGGTATCCTCCAGACCGTCAAGGTTGTCGACGAACTCGTGAAGGTCATGGG |
| | | CCGACACAAGCCCGAGAACATCGTTATCGAGATGGCCCGAGAGAACCAGACCACCC |
| | | AGAAGGGTCAGAAGAACTCCCGAGAGCGAATGAAGCGAATCGAAGAGGGTATCAAG |
| | | GAGCTCGGTTCCCAGATTCTCAAGGAGCACCCCGTCGAGAACACCCAGCTCCAGAA |
| | | CGAGAAACTCTACCTGTACTACCTCCAGAATGGCCGAGACATGTACGTTGACCAGG |
| | | AGCTCGACATCAACCGACTCTCCGACTACGACGTCGACCACATTGTTCCTCAGTCC |
| | | TTCCTCAAGGACGACTCCATCGACAACAAGGTTCTGACCCGATCTGACAAGAACCG |
| | | AGGTAAGTCCGACAACGTTCCCTCCGAAGAGGTCGTTAAGAAGATGAAGAACTACT |
| | | GGCGACAGCTTCTCAACGCCAAACTGATCACCCAGCGAAAGTTTGACAACCTCACC |
| | | AAGGCCGAGCGAGGTGGTCTGTCCGAGCTGGACAAGGCCGGCTTCATTAAGCGACA |
| | | GCTGGTCGAGACTCGACAGATCACCAAGCACGTCGCCCAGATCCTCGACTCCCGAA |
| | | TGAACACCAAGTACGACGAGAACGACAAGCTCATCCGGGAGGTCAAGGTCATCACC |
| | | CTGAAGTCTAAGCTTGTCTCCGACTTCCGAAAGGACTTCCAGTTCTACAAGGTCCG |
| | | AGAGATCAACAACTACCACCACGCCCACGACGCCTACCTCAACGCCGTTGTTGGTA |
| | | CCGCCCTCATCAAGAAGTATCCCAAGCTCGAGTCCGAGTTCGTTTACGGCGACTAC |
| | | AAGGTTTACGATGTCCGAAAGATGATTGCCAAGTCCGAGCAGGAGATCGGTAAGGC |
| | | CACCGCCAAGTACTTTTTCTACTCCAACATCATGAATTTCTTCAAGACCGAGATCA |
| | | CTCTCGCCAACGGTGAGATTCGAAAGCGACCCCTGATTGAGACTAATGGTGAGACT |
| | | GGTGAGATCGTCTGGGATAAGGGCCGAGACTTCGCCACCGTCCGAAAGGTCCTGTC |
| | | CATGCCCCAGGTCAACATTGTCAAGAAGACCGAGGTCCAGACCGGTGGCTTCTCCA |
| | | AGGAGTCCATTCTCCCCAAGCGAAACTCCGACAAACTCATCGCCCGTAAGAAGGAC |
| | | TGGGATCCGAAGAAGTACGGTGGTTTCGATTCTCCCACCGTTGCCTACTCCGTCCT |
| | | CGTTGTTGCTAAAGTCGAGAAGGGTAAGTCTAAGAAACTCAAGTCCGTGAAGGAGC |
| | | TACTCGGTATCACCATCATGGAGCGATCTTCTTTTGAGAAGAACCCCATTGACTTC |
| | | CTCGAGGCCAAGGGTTACAAAGAGGTCAAGAAGGACCTGATTATCAAGCTGCCCAA |
| | | GTACTCCCTCTTTGAGCTCGAGAACGGCCGAAAGCGAATGCTGGCTTCCGCTGGTG |
| | | AGCTGCAGAAGGGCAACGAGCTCGCTCTGCCCTCCAAGTACGTCAACTTCCTCTAC |
| | | CTGGCCTCCCACTACGAGAAGCTCAAGGGCTCCCCCGAGGACAACGAGCAGAAGCA |
| | | GCTGTTCGTTGAGCAGCACAAGCACTACCTCGACGAGATCATCGAGCAGATCTCCG |
| | | AGTTCTCCAAGCGAGTCATCCTCGCTGACGCCAACCTTGATAAGGTTCTCTCTGCT |
| | | TACAACAAGCACCGGGACAAGCCCATCCGAGAGCAGGCCGAGAATATCATCCACCT |
| | | CTTCACTCTCACCAACCTCGGCGCTCCTGCTGCCTTCAAGTACTTCGACACCACCA |
| | | TTGACCGAAAGAGGTACACCTCCACCAAGGAAGTCCTCGACGCCACCCTGATCCAC |
| | | CAGTCCATCACCGGCCTCTACGAAACCCGAATCGACCTCTCCCAGCTCGGCGGTGA |
| | | CTCTCGAGCCGACCCCAAGAAGAAGCGAAAAGTCTAAATATCCGAAGATCAAGAGC |
| | | GAAGCAAGTTGTAAGTCCAGGACATGTTTCCCGCCCACGCGAGTGATTTATAACAC |
| | | CTCTCTTTTTTGACACCCGCTCGCCTTGAAATTCATGTCACATAAATTATAGTCAA |
| | | CGACGTTTGAATAACTTGTCTTGTAGTTCGATGATGATCATATGATTACATTAATA |
| | | GTAATTACTGTATTTGATATATATACTAATTACAATAGTACATATTAGAACATACA |
| | | ATAGTTAGTGCCGTGAAGTGGCTTAAAATACCGCGAGTCGATTACGTAATATTATT |
| | | ACCTCTTGCCCATCGAACGTACAAGTACTCCTCTGTTCTCTCCTTCCTTTGCTTTG |
| | | TGCACGAAGAACTGCGGTCAGGTGACACAACTTTTTCCATCTCAGGGTGTGTCGCG |
| | | TGTGCTTCATCCAAACTTTAGTTGGGGTTCGGGTTCGCGCGAGATGATCACGTGCC |
| | | CTGATTTGGTGTCGTCCCCCGTCGCGCTGCGCACGTGATTTATTTATTTCCGGTGG |
| | | CTGCTGTCTACGCGGGGCCTTCTCTGCCCTTCTGTTTCAACCTTCGGGCGGTTCTC |
| | | GTAACCAGCAGTAGCAATCCATTTCGAAACTCAAAGAGCTAAAAACGTTAAACCTC |
| | | AGCAGTCGCTCGACGAATGGGCTGCGGTTGGGAAGCCCACGAGGCCTATAGCCAGA |
| | | GCCTCGAGTTGACAGGAGCCCAGACGCCTTTTCCAACGGCAACTTTTATATAAAAT |
| | | GGCAATGTATTCATGCAATTGCGGCCGTGTCAGGTTGGAGACACTGGACCACACTC |
| | | TCCATTGCTTCCTGAGGAGATGGATCATTGCTAGTGCATCTACGCGCAGCAATCCC |
| | | GCAAGCTCGACAACCGTAGATGGGCTTTGGTGGGCCAATCAATTACGCAACCCGCA |
| | | CGTTAAATTGTATGAGGAAGGAAGGCCACGGTACAAAGTGGGTGGTCTTCACCCAG |
| | | TGGTTGTTGGTGGCGTCATGCAGACCATGCATTGGGGATAGCACAGGGTTGGGGTG |
| | | TCTTGTGGACTCAATGGGTGAAAGGAGATGGAAAAGGGCGGTGAAAAGTGGTAGAA |
| | | TCGAAATCCCTGACGTCAATTTATAAAGTAAAATGCGTTTCTGCCATTTTGCTCCC |
| | | CTCCTTCTTTCGCAATCGCCTCCCCAAAAGTTGTCGTGGCAGTACACATGCTTGCA |
| | | TACAATGAAGCTAATCCGGCTTGCTCAGTAGTTGCTATATCCAGGCATGGTGTGAA |
| | | ACCCCTCAAAGTATATATAGGAGCGGTGAGCCCCAGTCTGGGGTCTTTTCTCTCCA |
| | | TCTCAAAACTACTTTCTCACAATGAAGAAGCTGATGAGTCCGTGAGGACGAAACGA |
| | | GTAAGCTCGTCCTTCTTTCGTCCAATGTCGGGTTTTAGAGCTAGAAATAGCAAGTT |
| | | AAAATAAGGCTAGTCCGTTATCAACTTGAAAAAGTGGCACCGAGTCGGTGCTTTTG |
| | | GCCGGCATGGTCCCAGCCTCCTCGCTGGCGCCGGCTGGGCAACATGCTTCGGCATG |
| | | GCGAATGGGACTAAACTTCGAGCTAATCCAGTAGCTTACGTTACCCAGGGGCAGGT |
| | | CAACTGGCTAGCCACGAGTCTGTCCCAGGTCGCAATTTAGTGTAATAAACAATATA |
| | | TATATTGAGTCTAAAGGGAATTGTAGCTATTGTGATTGTGTGATTTTCGTCTTGCT |
| | | GGTTCTTATTGTGTCCCATTCGTTTCATCCTGATGAGGACCCCTGGAACCGGTGTT |
| | | TTCTTAGTCTCTGCAATCGCTAGTCTTGTTGCTATGACAGTTGCGTCGACACTATT |
| | | CAGGTCATCTATCGGTTATTCTGATATTATAATACCTCCGGATCGATGTACCTGAT |
| | | TTATACTTGCAGCAATGTTTACTTCTTATCGCGATACACGAATGTGATACGGATCA |
| | | AAGTAAGCAGGACTACGATAAGATAACGAATGCGGTGCAGTCCATGTCGATTAGGT |
| | | ATAGATACATTTATTTTGTGTTATGTTACATTTTGGGGGGATACTGTCCTACTTGT |
| | | AGTACCTACTTGTAGTGGCGCGTCTATTCCTTTGCCCTCGGACGAGTGCTGGGGCG |
| | | TCGGTTTCCACTATCGGCGAGTACTTCTACACAGCCATCGGTCCAGACGGCCGCGC |
| | | TTCTGCGGGCGATTTGTGTACGCCCGACAGTCCCGGCTCCGGATCGGACGATTGCG |
| | | TCGCATCGACCCTGCGCCCAAGCTGCATCATCGAAATTGCCGTCAACCAAGCTCTG |
| | | ATAGAGTTGGTCAAGACCAATGCGGAGCATATACGCCCGGAGCCGCGGCGATCCTG |
| | | CAAGCTCCGGATGCCTCCGCTCGAAGTAGCGCGTCTGCTGCTCCATACAAGCCAAC |
| | | CACGGCCTCCAGAAGAAGATGTTGGCGACCTCGTATTGGGAATCCCCGAACATCGC |
| | | CTCGCTCCAGTCAATGACCGCTGTTATGCGGCCATTGTCCGTCAGGACATTGTTGG |
| | | AGCCGAAATCCGCGTGCACGAGGTGCCGGACTTCGGGGCAGTCCTCGGCCCAAAGC |
| | | ATCAGCTCATCGAGAGCCTGCGCGACGGACGCACTGACGGTGTCGTCCATCACAGT |
| | | TTGCCAGTGATACACATGGGGATCAGCAATCGCGCATATGAAATCACGCCATGTAG |
| | | TGTATTGACCGATTCCTTGCGGTCCGAATGGGCCGAACCCGCTCGTCTGGCTAAGA |
| | | TCGGCCGCAGCGATCGCATCCATGGCCTCCGCGACCGGCTGCAGAACAGCGGGCAG |
| | | TTCGGTTTCAGGCAGGTCTTGCAACGTGACACCCTGTGCACGGCGGGAGATGCAAT |
| | | AGGTCAGGCTCTCGCTGAATTCCCCAATGTCAAGCACTTCCGGAATCGGGAGCGCG |
| | | GCCGATGCAAAGTGCCGATAAACATAACGATCTTTGTAGAAACCATCGGCGCAGCT |
| | | ATTTACCCGCAGGACATATCCACGCCCTCCTACATCGAAGCTGAAAGCACGAGATT |
| | | CTTCGCCCTCCGAGAGCTGCATCAGGTCGGAGACGCTGTCGAACTTTTCGATCAGA |
| | | AACTTCTCGACAGACGTCGCGGTGAGTTCAGGCTTTTTCATATGGGTACCTGAGAA |
| | | CATTTTTGTGTCTAGGTGTTTGTGTTTGGACTGCGATCAGTGAAGAAAAGAAGAGG |
| | | AAAAATTGTGCAAGAAATTTTGCTTTCAAGACTTGGCTGATGCAGCAGGGTAACTC |
| | | TGGGACACAGACCTATGTTTGTGGTTAAACTCAATGCACGTGGTACGTGCGTGGAG |
| | | CGCTTACCCATCCAAGGGTGTGGACATGGAACCGACGGTCCGTGGAGTTGTGTAAT |
| | | GTCATTTTGGCGACTCTTGAAGCAAGGCTATAAAAAAATTGTGTGGCTTGAGTCTT |
| | | ATCGAGCTCGGTCACTACAAGAGTTAATCTTCCTGTCTCAGGCAGACAGGTCAGGC |
| | | AGGGTTACTTTTGGGTGTGCTGTAACTCACTGTATGGCCGTTAGTGCGCATAGACG |
| | | TTGTACATACTGGACCGAATTGTAGCGTGCTCAATAGGGCCAATAAAGCTATTGTA |
| | | GGGATCCGAATTTTCAGAACCTAATTTATCTGTTACCCGGCCTGTGGCTCGCACAG |
| | | CTTAAAAATGGTCAAACTTTCCCCTTCTTGTCTTTTTTTCCTCACATTCATCAGGT |
| | | TCTTGTCTTGATCTTTCAAGTGAGTATTAATTACCGACCTTGGTTCTTCATTGGGA |
| | | GAGCATTGGAAGCCGTGGTGCAGCAACCACAAAACGGTTCTTCCCCTTCGATACCT |
| | | TCTTGCCTGCCTTTCAATACAAGTCGGCTCGATTAGCGGTGGTCGCCCCCGCCAGC |
| | | GGAGAACATGGAACTAACCCAGAATGAGAGCTAAGTGGAGAAAGAAGAGAGTCAGA |
| | | CGACTCAAGCGAAAGCGCCGCAAGGTCCGAGCTCGATCCAAATAAGCGGTTTTTAA |
| | | CGGAGATTTAACACTAAATCGAAGAACTTTTCCCGTTTCATTTGCGAATGAGCTCG |
| | | TTAACAAAATCCCCCAGTTTTTTTATCCAGCTGTAAGGATTGACATTAGTAATGAA |
| | | TTATTGTTTGGTATATTTAAATCTGTAGTTCCTTTCTGTCCGTGTCGGCAACTGTC |
| | | GTACTCGTGATTTACTTGTATTGACGAATACTTACTGTAGCGCACTCTGCTGCTAC |
| | | TGGTCGTAAGGATGTGCTATTTCGGTGTATGGTGGGTTTTTTGGGGGTCGGAACCG |
| | | AAGACTGTTACACGGGCACGGCTCGTTGTGTACACGCACAGAGCTCTTGCGAGTCA |
| | | TGTTGTAGCTAGCTCGTCGTGTTCAGGAACTGTTCGATGGTTCGGAGAGAGTCGCC |
| | | GCCCAGAACATACGCGCACCGATGTCAGCAGACAGCCTTATTACAAGTATATTCAA |
| | | GCAAGTATATCCGTAGGGTGCGGGTGATTTGGATCTAAGGTTCGTACTCAACACTC |
| | | ACGAGCAGCTTGCCTATGTTACATCCTTTTATCAGACATAACATAATTGGAGTTTA |
| | | CTTACACACGGGGTGTACCTGTATGAGCACCACCTACAATTGTAGCACTGGTACTT |
| | | GTACAAAGAATTTATTCGTACGAATCACAGGGACGGCCGCCCTCACCGAACCAGCG |
| | | AATACCTCAGCGGTCCCCTGCAGTGACTCAACAAAGCGATATGAACATCTTGCGAT |
| | | GGTATCCTGCTGATAGTTTTTACTGTACAAACACCTGTGTAGCTCCTTCTAGCATT |
| | | TTTAAGTTATTCACACCTCAAGGGGAGGGATAAATTAAATAAATTCCAAAAGCGAA |
| | | GATCGAGAAACTAAATTAAAATTCCAAAAACGAAGTTGGAACACAACCCCCCGAAA |
| | | AAAAACAACAAACAAAAAACCCAACAAAATAAACAAAAACAAAATAAATATATAAC |
| | | TACCAGTATCTGACTAAAAGTTCAAATACTCGTACTTACAACAAATAGAAATGAGC |
| | | CGGCCAAAATTCTGCAGAAAAAAATTTCAAACAAGTACTGGTATAATTAAATTAAA |
| | | AAACACATCAAAGTATCATAACGTTAGTTATTTTATTTTATTTAATAAAAGAAAAC |
| | | AACAAGATGGGCTCAAAACTTTCAACTTATACGATACATACCAAATAACAATTTAG |
| | | TATTTATCTAAGTGCTTTTCGTAGATAATGGAATACAAATGGATATCCAGAGTATA |
| | | CACATGGATAGTATACACTGACACGACAATTCTGTATCTCTTTATGTTAACTACTG |
| | | TGAGGCATTAAATAGAGCTTGATATATAAAATGTTACATTTCACAGTCTGAACTTT |
| | | TGCAGATTACCTAATTTGGTAAGATATTAATTATGAACTGAAAGTTGATGGCATCC |
| | | CTAAATTTGATGAAAGATGAAATTGTAAATGAGGTGGTAAAAGAGCTACAGTCGTT |
| | | TTGTTTTGAGATACCATCATCTCTAACGAAATATCTATTAAAAATCTCAGTGTGAT |
| | | CATGAGTCATTGCCATCCTGGAAAATGTCATCATGGCTGATATTTCTAACTGTTTA |
| | | CTTGAGATAAATATATATTTACAAGAACTTCCCTTGAAATTAATTTAGATATAAAA |
| | | TGTTTGCGGGCAAGTTACTACGAGGAATAAATTATATCTAGA |
| MB7549 | 8 | GGTTCCGCTTCCTCGCTCACTGACTCGCTGCGCTCGGTCGTTCGGCTGCGGCGAGC |
| | | GGTATCAGCTCACTCAAAGGCGGTAATACGGTTATCCACAGAATCAGGGGATAACG |
| | | CAGGAAAGAACATGTGAGCAAAAGGCCAGCAAAAGGCCAGGAACCGTAAAAAGGCC |
| | | GCGTTGCTGGCGTTTTTCCATAGGCTCCGCCCCCCTGACGAGCATCACAAAAATCG |
| | | ACGCTCAAGTCAGAGGTGGCGAAACCCGACAGGACTATAAAGATACCAGGCGTTTC |
| | | CCCCTGGAAGCTCCCTCGTGCGCTCTCCTGTTCCGACCCTGCCGCTTACCGGATAC |
| | | CTGTCCGCCTTTCTCCCTTCGGGAAGCGTGGCGCTTTCTCATAGCTCACGCTGTAG |
| | | GTATCTCAGTTCGGTGTAGGTCGTTCGCTCCAAGCTGGGCTGTGTGCACGAACCCC |
| | | CCGTTCAGCCCGACCGCTGCGCCTTATCCGGTAACTATCGTCTTGAGTCCAACCCG |
| | | GTAAGACACGACTTATCGCCACTGGCAGCAGCCACTGGTAACAGGATTAGCAGAGC |
| | | GAGGTATGTAGGCGGTGCTACAGAGTTCTTGAAGTGGTGGCCTAACTACGGCTACA |
| | | CTAGAAGGACAGTATTTGGTATCTGCGCTCTGCTGAAGCCAGTTACCTTCGGAAAA |
| | | AGAGTTGGTAGCTCTTGATCCGGCAAACAAACCACCGCTGGTAGCGGTGGTTTTTT |
| | | TGTTTGCAAGCAGCAGATTACGCGCAGAAAAAAAGGATCTCAAGAAGATCCTTTGA |
| | | TCTTTTCTACGGGGTCTGACGCTCAGTGGAACGAAAACTCACGTTAAGGGATTTTG |
| | | GTCATGAGATTATCAAAAAGGATCTTCACCTAGATCCTTTTAAATTAAAAATGAAG |
| | | TTTTAAATCAATCTAAAGTATATATGAGTAAACTTGGTCTGACAGTTACCAATGCT |
| | | TAATCAGTGAGGCACCTATCTCAGCGATCTGTCTATTTCGTTCATCCATAGTTGCC |
| | | TGACTCCCCGTCGTGTAGATAACTACGATACGGGAGGGCTTACCATCTGGCCCCAG |
| | | TGCTGCAATGATACCGCGAGACCCACGCTCACCGGCTCCAGATTTATCAGCAATAA |
| | | ACCAGCCAGCCGGAAGGGCCGAGCGCAGAAGTGGTCCTGCAACTTTATCCGCCTCC |
| | | ATCCAGTCTATTAATTGTTGCCGGGAAGCTAGAGTAAGTAGTTCGCCAGTTAATAG |
| | | TTTGCGCAACGTTGTTGCCATTGCTGCAGGCATCGTGGTGTCACGCTCGTCGTTTG |
| | | GTATGGCTTCATTCAGCTCCGGTTCCCAACGATCAAGGCGAGTTACATGATCCCCC |
| | | ATGTTGTGCAAAAAAGCGGTTAGCTCCTTCGGTCCTCCGATCGTTGTCAGAAGTAA |
| | | GTTGGCCGCAGTGTTATCACTCATGGTTATGGCAGCACTGCATAATTCTCTTACTG |
| | | TCATGCCATCCGTAAGATGCTTTTCTGTGACTGGTGAGTACTCAACCAAGTCATTC |
| | | TGAGAATAGTGTATGCGGCGACCGAGTTGCTCTTGCCCGGCGTCAACACGGGATAA |
| | | TACCGCGCCACATAGCAGAACTTTAAAAGTGCTCATCATTGGAAAACGTTCTTCGG |
| | | GGCGAAAACTCTCAAGGATCTTACCGCTGTTGAGATCCAGTTCGATGTAACCCACT |
| | | CGTGCACCCAACTGATCTTCAGCATCTTTTACTTTCACCAGCGTTTCTGGGTGAGC |
| | | AAAAACAGGAAGGCAAAATGCCGCAAAAAAGGGAATAAGGGCGACACGGAAATGTT |
| | | GAATACTCATACTCTTCCTTTTTCAATATTATTGAAGCATTTATCAGGGTTATTGT |
| | | CTCATGAGCGGATACATATTTGAATGTATTTAGAAAAATAAACAAATAGGGGTTCC |
| | | GCGCACATTTCCCCGAAAAGTGCCACCTGACGTCTAAGAAACCATTATTATCATGA |
| | | CATTAACCTATAAAAATAGGCGTATCACGAGGCCCTTTCGTCTGGCCTAGGAAGCG |
| | | ACTTCCAATCGCTTTGCATATCCAGTACCACACCCACAGGCGTTTGTGCTACTCTA |
| | | CTGATAGCAATAGATGCGTCATAATTGGTTGGCCCGCTGAGCCTCCACAGGATACT |
| | | ATTGCACATACCCTGGTCATGTGCAGATCAGCTCATTTGTGGAGACTCTGGAGTAA |
| | | CTTAGACGACGCCTGGTTCAATTGCCGCAATGTGCGCCCACGCAGATAATGTATTG |
| | | AGGGGTGGAGCGCCTCTTGGGGACTTGCTGTACTTGTACGGGATATTAAACGCACT |
| | | CAGCAAGACCATGACGTAAAACACACCTACTGTACGATACGTACTGTAGGTATTGT |
| | | ACTCGTACCCGGTACTACAAATAGTACGATACTATACGGAGTGTATTTGTACCTTG |
| | | ATATACGACTGGCGGAGTGAAGAGAAGGAGTTGAACAAGACCAGATGGGGATATCA |
| | | GCCCCAGTGCTTTGTATTACAAGTACGAGTACTTAATAGATACTGTAAGGCTATTG |
| | | ATACGGATGGCAGTAAGTCATTGAGTAAGCAATTGTGGCCCAGCATCTCCCCTACG |
| | | TACTTGTACCATACCCCATGGAGACACCAATGGTCTTTCACGCACACTGTCGTGTG |
| | | CTGTATCGCAGAATCGGGTGTCCAACCAAATGCCGTTACCCCCACGTCACAGCCGA |
| | | TAGACAGATACACCATCAATACCAGCAGGTTGTATCATGCGGTTGGCTGAAGGTAA |
| | | GCTGATTGGTCTAAAAACTGTAGCTGTCCTAATTCAACGAGCGCTATTTGGGGCCA |
| | | ACCACCTCGGCCAAGCGGCCTTTAATCTGCGTGCCCCAGAGGCGTCTAATGAGGCT |
| | | CTGGCCGCCACTGTAGGAGTGTTTCTCTGTGCGCACACGCAGTTTTGAGTTTGGGC |
| | | GACTTTCCCTTTTTCCCAATTGCGTACACACACAGCTCCGAGCTAAGCGCTGTCCT |
| | | TGAACCTTCTCCCTCTTTTCCCTCTTTTTCTCTTCCCCTTCCCCTCCTCCACATTA |
| | | AGGCCAAATCCTGAATTGCACCAACTAGTACAACGACAACAATGGACAAGAAGTAC |
| | | TCCATCGGTTTGGACATTGGTACTAACTCTGTCGGCTGGGCCGTCATCACCGACGA |
| | | GTACAAGGTTCCCTCCAAGAAGTTCAAGGTCCTTGGCAACACCGACCGACACTCTA |
| | | TCAAGAAGAACCTGATCGGTGCTCTGCTGTTCGACTCTGGCGAGACTGCCGAGGCC |
| | | ACCCGACTGAAGCGAACCGCTCGACGCCGATACACCCGACGAAAGAACCGAATCTG |
| | | TTACCTCCAGGAGATCTTCAGCAACGAGATGGCTAAGGTCGACGACTCCTTCTTCC |
| | | ACCGACTCGAGGAGTCTTTCCTGGTCGAAGAGGATAAGAAGCACGAGCGACACCCC |
| | | ATCTTCGGCAACATTGTTGATGAGGTTGCCTACCATGAGAAGTACCCCACCATCTA |
| | | CCACCTCCGAAAGAAGCTCGTCGACTCCACTGACAAGGCTGACCTCCGACTCATCT |
| | | ACCTTGCTCTCGCCCACATGATCAAGTTCCGAGGTCACTTCCTCATTGAGGGTGAT |
| | | CTCAACCCCGACAACTCCGACGTTGACAAGCTGTTCATCCAGCTCGTCCAGACCTA |
| | | CAACCAGCTCTTTGAGGAGAACCCTATCAACGCTTCTGGTGTTGACGCCAAGGCCA |
| | | TTCTCTCCGCCCGACTCTCTAAGTCCCGACGACTCGAGAACCTCATTGCCCAGCTG |
| | | CCCGGCGAGAAGAAGAACGGCCTCTTCGGTAACCTGATTGCTCTCTCTCTTGGTCT |
| | | GACCCCCAACTTCAAGTCCAACTTTGACCTCGCCGAGGACGCCAAGCTCCAGCTGT |
| | | CCAAGGACACCTACGATGACGATCTGGACAACCTCCTGGCCCAGATCGGTGACCAG |
| | | TACGCCGATCTCTTCCTTGCCGCCAAGAACCTCTCCGACGCCATCCTGCTCTCCGA |
| | | CATCCTCCGAGTCAACACCGAGATTACCAAGGCTCCTCTGTCTGCCTCTATGATCA |
| | | AGCGATACGACGAGCACCACCAGGATCTCACTCTTCTCAAGGCTCTCGTCCGACAG |
| | | CAGCTCCCCGAGAAGTACAAGGAGATTTTCTTTGACCAGTCCAAGAACGGTTACGC |
| | | TGGCTACATTGACGGTGGTGCTTCCCAGGAAGAGTTTTACAAGTTCATCAAGCCTA |
| | | TTCTGGAGAAGATGGACGGTACCGAGGAGCTGCTCGTCAAGCTCAACCGAGAGGAC |
| | | CTCCTTCGAAAGCAGCGAACCTTCGATAACGGCTCCATCCCCCACCAGATCCACCT |
| | | GGGTGAGCTCCACGCCATTCTCCGAAGACAAGAGGACTTCTACCCCTTCCTAAAGG |
| | | ATAACCGAGAGAAGATCGAGAAGATTCTCACCTTCCGAATCCCCTACTACGTCGGT |
| | | CCCCTCGCTCGAGGTAACTCCCGATTTGCTTGGATGACCCGAAAGTCCGAGGAGAC |
| | | TATCACCCCCTGGAACTTTGAAGAGGTAGTCGACAAGGGTGCCTCCGCCCAGTCTT |
| | | TCATTGAGCGGATGACCAACTTCGATAAGAACCTCCCCAACGAGAAGGTCCTTCCC |
| | | AAGCACTCTCTCCTCTACGAGTACTTCACCGTCTACAACGAGCTGACCAAGGTCAA |
| | | GTACGTTACCGAGGGCATGCGAAAGCCCGCTTTCCTCTCTGGTGAGCAGAAGAAGG |
| | | CCATTGTCGACCTCCTGTTCAAGACTAACCGAAAAGTCACCGTCAAGCAGCTCAAG |
| | | GAAGACTACTTCAAGAAGATTGAGTGCTTCGACTCCGTCGAGATTTCCGGTGTCGA |
| | | GGACCGATTCAACGCCTCCCTCGGCACCTACCACGATCTTCTGAAGATCATCAAGG |
| | | ACAAGGACTTTCTTGATAACGAGGAGAACGAGGACATTCTCGAGGACATCGTCCTC |
| | | ACCCTCACCCTTTTCGAGGATCGAGAGATGATCGAGGAGCGACTCAAGACCTACGC |
| | | CCATCTCTTCGACGACAAGGTCATGAAGCAACTCAAGCGACGACGATACACTGGCT |
| | | GGGGCCGACTTTCCCGAAAGCTCATCAACGGCATCCGAGACAAGCAGTCTGGCAAG |
| | | ACCATCCTGGACTTCCTGAAGTCCGACGGTTTCGCCAACCGAAACTTCATGCAGCT |
| | | CATCCACGACGACTCTCTTACCTTCAAAGAGGATATCCAGAAGGCCCAGGTTTCTG |
| | | GCCAGGGCGACTCCCTCCACGAGCACATTGCCAACCTCGCCGGATCCCCCGCCATC |
| | | AAAAAGGGTATCCTCCAGACCGTCAAGGTTGTCGACGAACTCGTGAAGGTCATGGG |
| | | CCGACACAAGCCCGAGAACATCGTTATCGAGATGGCCCGAGAGAACCAGACCACCC |
| | | AGAAGGGTCAGAAGAACTCCCGAGAGCGAATGAAGCGAATCGAAGAGGGTATCAAG |
| | | GAGCTCGGTTCCCAGATTCTCAAGGAGCACCCCGTCGAGAACACCCAGCTCCAGAA |
| | | CGAGAAACTCTACCTGTACTACCTCCAGAATGGCCGAGACATGTACGTTGACCAGG |
| | | AGCTCGACATCAACCGACTCTCCGACTACGACGTCGACCACATTGTTCCTCAGTCC |
| | | TTCCTCAAGGACGACTCCATCGACAACAAGGTTCTGACCCGATCTGACAAGAACCG |
| | | AGGTAAGTCCGACAACGTTCCCTCCGAAGAGGTCGTTAAGAAGATGAAGAACTACT |
| | | GGCGACAGCTTCTCAACGCCAAACTGATCACCCAGCGAAAGTTTGACAACCTCACC |
| | | AAGGCCGAGCGAGGTGGTCTGTCCGAGCTGGACAAGGCCGGCTTCATTAAGCGACA |
| | | GCTGGTCGAGACTCGACAGATCACCAAGCACGTCGCCCAGATCCTCGACTCCCGAA |
| | | TGAACACCAAGTACGACGAGAACGACAAGCTCATCCGGGAGGTCAAGGTCATCACC |
| | | CTGAAGTCTAAGCTTGTCTCCGACTTCCGAAAGGACTTCCAGTTCTACAAGGTCCG |
| | | AGAGATCAACAACTACCACCACGCCCACGACGCCTACCTCAACGCCGTTGTTGGTA |
| | | CCGCCCTCATCAAGAAGTATCCCAAGCTCGAGTCCGAGTTCGTTTACGGCGACTAC |
| | | AAGGTTTACGATGTCCGAAAGATGATTGCCAAGTCCGAGCAGGAGATCGGTAAGGC |
| | | CACCGCCAAGTACTTTTTCTACTCCAACATCATGAATTTCTTCAAGACCGAGATCA |
| | | CTCTCGCCAACGGTGAGATTCGAAAGCGACCCCTGATTGAGACTAATGGTGAGACT |
| | | GGTGAGATCGTCTGGGATAAGGGCCGAGACTTCGCCACCGTCCGAAAGGTCCTGTC |
| | | CATGCCCCAGGTCAACATTGTCAAGAAGACCGAGGTCCAGACCGGTGGCTTCTCCA |
| | | AGGAGTCCATTCTCCCCAAGCGAAACTCCGACAAACTCATCGCCCGTAAGAAGGAC |
| | | TGGGATCCGAAGAAGTACGGTGGTTTCGATTCTCCCACCGTTGCCTACTCCGTCCT |
| | | CGTTGTTGCTAAAGTCGAGAAGGGTAAGTCTAAGAAACTCAAGTCCGTGAAGGAGC |
| | | TACTCGGTATCACCATCATGGAGCGATCTTCTTTTGAGAAGAACCCCATTGACTTC |
| | | CTCGAGGCCAAGGGTTACAAAGAGGTCAAGAAGGACCTGATTATCAAGCTGCCCAA |
| | | GTACTCCCTCTTTGAGCTCGAGAACGGCCGAAAGCGAATGCTGGCTTCCGCTGGTG |
| | | AGCTGCAGAAGGGCAACGAGCTCGCTCTGCCCTCCAAGTACGTCAACTTCCTCTAC |
| | | CTGGCCTCCCACTACGAGAAGCTCAAGGGCTCCCCCGAGGACAACGAGCAGAAGCA |
| | | GCTGTTCGTTGAGCAGCACAAGCACTACCTCGACGAGATCATCGAGCAGATCTCCG |
| | | AGTTCTCCAAGCGAGTCATCCTCGCTGACGCCAACCTTGATAAGGTTCTCTCTGCT |
| | | TACAACAAGCACCGGGACAAGCCCATCCGAGAGCAGGCCGAGAATATCATCCACCT |
| | | CTTCACTCTCACCAACCTCGGCGCTCCTGCTGCCTTCAAGTACTTCGACACCACCA |
| | | TTGACCGAAAGAGGTACACCTCCACCAAGGAAGTCCTCGACGCCACCCTGATCCAC |
| | | CAGTCCATCACCGGCCTCTACGAAACCCGAATCGACCTCTCCCAGCTCGGCGGTGA |
| | | CTCTCGAGCCGACCCCAAGAAGAAGCGAAAAGTCTAAATATCCGAAGATCAAGAGC |
| | | GAAGCAAGTTGTAAGTCCAGGACATGTTTCCCGCCCACGCGAGTGATTTATAACAC |
| | | CTCTCTTTTTTGACACCCGCTCGCCTTGAAATTCATGTCACATAAATTATAGTCAA |
| | | CGACGTTTGAATAACTTGTCTTGTAGTTCGATGATGATCATATGATTACATTAATA |
| | | GTAATTACTGTATTTGATATATATACTAATTACAATAGTACATATTAGAACATACA |
| | | ATAGTTAGTGCCGTGAAGTGGCTTAAAATACCGCGAGTCGATTACGTAATATTATT |
| | | ACCTCTTGCCCATCGAACGTACAAGTACTCCTCTGTTCTCTCCTTCCTTTGCTTTG |
| | | TGCACGAAGAACTGCGGTCAGGTGACACAACTTTTTCCATCTCAGGGTGTGTCGCG |
| | | TGTGCTTCATCCAAACTTTAGTTGGGGTTCGGGTTCGCGCGAGATGATCACGTGCC |
| | | CTGATTTGGTGTCGTCCCCCGTCGCGCTGCGCACGTGATTTATTTATTTCCGGTGG |
| | | CTGCTGTCTACGCGGGGCCTTCTCTGCCCTTCTGTTTCAACCTTCGGGCGGTTCTC |
| | | GTAACCAGCAGTAGCAATCCATTTCGAAACTCAAAGAGCTAAAAACGTTAAACCTC |
| | | AGCAGTCGCTCGACGAATGGGCTGCGGTTGGGAAGCCCACGAGGCCTATAGCCAGA |
| | | GCCTCGAGTTGACAGGAGCCCAGACGCCTTTTCCAACGGCAACTTTTATATAAAAT |
| | | GGCAATGTATTCATGCAATTGCGGCCGTGTCAGGTTGGAGACACTGGACCACACTC |
| | | TCCATTGCTTCCTGAGGAGATGGATCATTGCTAGTGCATCTACGCGCAGCAATCCC |
| | | GCAAGCTCGACAACCGTAGATGGGCTTTGGTGGGCCAATCAATTACGCAACCCGCA |
| | | CGTTAAATTGTATGAGGAAGGAAGGCCACGGTACAAAGTGGGTGGTCTTCACCCAG |
| | | TGGTTGTTGGTGGCGTCATGCAGACCATGCATTGGGGATAGCACAGGGTTGGGGTG |
| | | TCTTGTGGACTCAATGGGTGAAAGGAGATGGAAAAGGGCGGTGAAAAGTGGTAGAA |
| | | TCGAAATCCCTGACGTCAATTTATAAAGTAAAATGCGTTTCTGCCATTTTGCTCCC |
| | | CTCCTTCTTTCGCAATCGCCTCCCCAAAAGTTGTCGTGGCAGTACACATGCTTGCA |
| | | TACAATGAAGCTAATCCGGCTTGCTCAGTAGTTGCTATATCCAGGCATGGTGTGAA |
| | | ACCCCTCAAAGTATATATAGGAGCGGTGAGCCCCAGTCTGGGGTCTTTTCTCTCCA |
| | | TCTCAAAACTACTTTCTCACAATGGCCGCCCTGATGAGTCCGTGAGGACGAAACGA |
| | | GTAAGCTCGTCGGCGGCTAGCCACAGCGAGAGTTTTAGAGCTAGAAATAGCAAGTT |
| | | AAAATAAGGCTAGTCCGTTATCAACTTGAAAAAGTGGCACCGAGTCGGTGCTTTTG |
| | | GCCGGCATGGTCCCAGCCTCCTCGCTGGCGCCGGCTGGGCAACATGCTTCGGCATG |
| | | GCGAATGGGACTAAACTTCGAGCTAATCCAGTAGCTTACGTTACCCAGGGGCAGGT |
| | | CAACTGGCTAGCCACGAGTCTGTCCCAGGTCGCAATTTAGTGTAATAAACAATATA |
| | | TATATTGAGTCTAAAGGGAATTGTAGCTATTGTGATTGTGTGATTTTCGTCTTGCT |
| | | GGTTCTTATTGTGTCCCATTCGTTTCATCCTGATGAGGACCCCTGGAACCGGTGTT |
| | | TTCTTAGTCTCTGCAATCGCTAGTCTTGTTGCTATGACAGTTGCGTCGACACTATT |
| | | CAGGTCATCTATCGGTTATTCTGATATTATAATACCTCCGGATCGATGTACCTGAT |
| | | TTATACTTGCAGCAATGTTTACTTCTTATCGCGATACACGAATGTGATACGGATCA |
| | | AAGTAAGCAGGACTACGATAAGATAACGAATGCGGTGCAGTCCATGTCGATTAGGT |
| | | ATAGATACATTTATTTTGTGTTATGTTACATTTTGGGGGGATACTGTCCTACTTGT |
| | | AGTACCTACTTGTAGTGGCGCGTCTATTCCTTTGCCCTCGGACGAGTGCTGGGGCG |
| | | TCGGTTTCCACTATCGGCGAGTACTTCTACACAGCCATCGGTCCAGACGGCCGCGC |
| | | TTCTGCGGGCGATTTGTGTACGCCCGACAGTCCCGGCTCCGGATCGGACGATTGCG |
| | | TCGCATCGACCCTGCGCCCAAGCTGCATCATCGAAATTGCCGTCAACCAAGCTCTG |
| | | ATAGAGTTGGTCAAGACCAATGCGGAGCATATACGCCCGGAGCCGCGGCGATCCTG |
| | | CAAGCTCCGGATGCCTCCGCTCGAAGTAGCGCGTCTGCTGCTCCATACAAGCCAAC |
| | | CACGGCCTCCAGAAGAAGATGTTGGCGACCTCGTATTGGGAATCCCCGAACATCGC |
| | | CTCGCTCCAGTCAATGACCGCTGTTATGCGGCCATTGTCCGTCAGGACATTGTTGG |
| | | AGCCGAAATCCGCGTGCACGAGGTGCCGGACTTCGGGGCAGTCCTCGGCCCAAAGC |
| | | ATCAGCTCATCGAGAGCCTGCGCGACGGACGCACTGACGGTGTCGTCCATCACAGT |
| | | TTGCCAGTGATACACATGGGGATCAGCAATCGCGCATATGAAATCACGCCATGTAG |
| | | TGTATTGACCGATTCCTTGCGGTCCGAATGGGCCGAACCCGCTCGTCTGGCTAAGA |
| | | TCGGCCGCAGCGATCGCATCCATGGCCTCCGCGACCGGCTGCAGAACAGCGGGCAG |
| | | TTCGGTTTCAGGCAGGTCTTGCAACGTGACACCCTGTGCACGGCGGGAGATGCAAT |
| | | AGGTCAGGCTCTCGCTGAATTCCCCAATGTCAAGCACTTCCGGAATCGGGAGCGCG |
| | | GCCGATGCAAAGTGCCGATAAACATAACGATCTTTGTAGAAACCATCGGCGCAGCT |
| | | ATTTACCCGCAGGACATATCCACGCCCTCCTACATCGAAGCTGAAAGCACGAGATT |
| | | CTTCGCCCTCCGAGAGCTGCATCAGGTCGGAGACGCTGTCGAACTTTTCGATCAGA |
| | | AACTTCTCGACAGACGTCGCGGTGAGTTCAGGCTTTTTCATATGGGTACCTGAGAA |
| | | CATTTTTGTGTCTAGGTGTTTGTGTTTGGACTGCGATCAGTGAAGAAAAGAAGAGG |
| | | AAAAATTGTGCAAGAAATTTTGCTTTCAAGACTTGGCTGATGCAGCAGGGTAACTC |
| | | TGGGACACAGACCTATGTTTGTGGTTAAACTCAATGCACGTGGTACGTGCGTGGAG |
| | | CGCTTACCCATCCAAGGGTGTGGACATGGAACCGACGGTCCGTGGAGTTGTGTAAT |
| | | GTCATTTTGGCGACTCTTGAAGCAAGGCTATAAAAAAATTGTGTGGCTTGAGTCTT |
| | | ATCGAGCTCGGTCACTACAAGAGTTAATCTTCCTGTCTCAGGCAGACAGGTCAGGC |
| | | AGGGTTACTTTTGGGTGTGCTGTAACTCACTGTATGGCCGTTAGTGCGCATAGACG |
| | | TTGTACATACTGGACCGAATTGTAGCGTGCTCAATAGGGCCAATAAAGCTATTGTA |
| | | GGGATCCGAATTTTCAGAACCTAATTTATCTGTTACCCGGCCTGTGGCTCGCACAG |
| | | CTTAAAAATGGTCAAACTTTCCCCTTCTTGTCTTTTTTTCCTCACATTCATCAGGT |
| | | TCTTGTCTTGATCTTTCAAGTGAGTATTAATTACCGACCTTGGTTCTTCATTGGGA |
| | | GAGCATTGGAAGCCGTGGTGCAGCAACCACAAAACGGTTCTTCCCCTTCGATACCT |
| | | TCTTGCCTGCCTTTCAATACAAGTCGGCTCGATTAGCGGTGGTCGCCCCCGCCAGC |
| | | GGAGAACATGGAACTAACCCAGAATGAGAGCTAAGTGGAGAAAGAAGAGAGTCAGA |
| | | CGACTCAAGCGAAAGCGCCGCAAGGTCCGAGCTCGATCCAAATAAGCGGTTTTTAA |
| | | CGGAGATTTAACACTAAATCGAAGAACTTTTCCCGTTTCATTTGCGAATGAGCTCG |
| | | TTAACAAAATCCCCCAGTTTTTTTATCCAGCTGTAAGGATTGACATTAGTAATGAA |
| | | TTATTGTTTGGTATATTTAAATCTGTAGTTCCTTTCTGTCCGTGTCGGCAACTGTC |
| | | GTACTCGTGATTTACTTGTATTGACGAATACTTACTGTAGCGCACTCTGCTGCTAC |
| | | TGGTCGTAAGGATGTGCTATTTCGGTGTATGGTGGGTTTTTTGGGGGTCGGAACCG |
| | | AAGACTGTTACACGGGCACGGCTCGTTGTGTACACGCACAGAGCTCTTGCGAGTCA |
| | | TGTTGTAGCTAGCTCGTCGTGTTCAGGAACTGTTCGATGGTTCGGAGAGAGTCGCC |
| | | GCCCAGAACATACGCGCACCGATGTCAGCAGACAGCCTTATTACAAGTATATTCAA |
| | | GCAAGTATATCCGTAGGGTGCGGGTGATTTGGATCTAAGGTTCGTACTCAACACTC |
| | | ACGAGCAGCTTGCCTATGTTACATCCTTTTATCAGACATAACATAATTGGAGTTTA |
| | | CTTACACACGGGGTGTACCTGTATGAGCACCACCTACAATTGTAGCACTGGTACTT |
| | | GTACAAAGAATTTATTCGTACGAATCACAGGGACGGCCGCCCTCACCGAACCAGCG |
| | | AATACCTCAGCGGTCCCCTGCAGTGACTCAACAAAGCGATATGAACATCTTGCGAT |
| | | GGTATCCTGCTGATAGTTTTTACTGTACAAACACCTGTGTAGCTCCTTCTAGCATT |
| | | TTTAAGTTATTCACACCTCAAGGGGAGGGATAAATTAAATAAATTCCAAAAGCGAA |
| | | GATCGAGAAACTAAATTAAAATTCCAAAAACGAAGTTGGAACACAACCCCCCGAAA |
| | | AAAAACAACAAACAAAAAACCCAACAAAATAAACAAAAACAAAATAAATATATAAC |
| | | TACCAGTATCTGACTAAAAGTTCAAATACTCGTACTTACAACAAATAGAAATGAGC |
| | | CGGCCAAAATTCTGCAGAAAAAAATTTCAAACAAGTACTGGTATAATTAAATTAAA |
| | | AAACACATCAAAGTATCATAACGTTAGTTATTTTATTTTATTTAATAAAAGAAAAC |
| | | AACAAGATGGGCTCAAAACTTTCAACTTATACGATACATACCAAATAACAATTTAG |
| | | TATTTATCTAAGTGCTTTTCGTAGATAATGGAATACAAATGGATATCCAGAGTATA |
| | | CACATGGATAGTATACACTGACACGACAATTCTGTATCTCTTTATGTTAACTACTG |
| | | TGAGGCATTAAATAGAGCTTGATATATAAAATGTTACATTTCACAGTCTGAACTTT |
| | | TGCAGATTACCTAATTTGGTAAGATATTAATTATGAACTGAAAGTTGATGGCATCC |
| | | CTAAATTTGATGAAAGATGAAATTGTAAATGAGGTGGTAAAAGAGCTACAGTCGTT |
| | | TTGTTTTGAGATACCATCATCTCTAACGAAATATCTATTAAAAATCTCAGTGTGAT |
| | | CATGAGTCATTGCCATCCTGGAAAATGTCATCATGGCTGATATTTCTAACTGTTTA |
| | | CTTGAGATAAATATATATTTACAAGAACTTCCCTTGAAATTAATTTAGATATAAAA |
| | | TGTTTGCGGGCAAGTTACTACGAGGAATAAATTATATCTAGA |
| MB7550 | 9 | GGTTCCGCTTCCTCGCTCACTGACTCGCTGCGCTCGGTCGTTCGGCTGCGGCGAGC |
| | | GGTATCAGCTCACTCAAAGGCGGTAATACGGTTATCCACAGAATCAGGGGATAACG |
| | | CAGGAAAGAACATGTGAGCAAAAGGCCAGCAAAAGGCCAGGAACCGTAAAAAGGCC |
| | | GCGTTGCTGGCGTTTTTCCATAGGCTCCGCCCCCCTGACGAGCATCACAAAAATCG |
| | | ACGCTCAAGTCAGAGGTGGCGAAACCCGACAGGACTATAAAGATACCAGGCGTTTC |
| | | CCCCTGGAAGCTCCCTCGTGCGCTCTCCTGTTCCGACCCTGCCGCTTACCGGATAC |
| | | CTGTCCGCCTTTCTCCCTTCGGGAAGCGTGGCGCTTTCTCATAGCTCACGCTGTAG |
| | | GTATCTCAGTTCGGTGTAGGTCGTTCGCTCCAAGCTGGGCTGTGTGCACGAACCCC |
| | | CCGTTCAGCCCGACCGCTGCGCCTTATCCGGTAACTATCGTCTTGAGTCCAACCCG |
| | | GTAAGACACGACTTATCGCCACTGGCAGCAGCCACTGGTAACAGGATTAGCAGAGC |
| | | GAGGTATGTAGGCGGTGCTACAGAGTTCTTGAAGTGGTGGCCTAACTACGGCTACA |
| | | CTAGAAGGACAGTATTTGGTATCTGCGCTCTGCTGAAGCCAGTTACCTTCGGAAAA |
| | | AGAGTTGGTAGCTCTTGATCCGGCAAACAAACCACCGCTGGTAGCGGTGGTTTTTT |
| | | TGTTTGCAAGCAGCAGATTACGCGCAGAAAAAAAGGATCTCAAGAAGATCCTTTGA |
| | | TCTTTTCTACGGGGTCTGACGCTCAGTGGAACGAAAACTCACGTTAAGGGATTTTG |
| | | GTCATGAGATTATCAAAAAGGATCTTCACCTAGATCCTTTTAAATTAAAAATGAAG |
| | | TTTTAAATCAATCTAAAGTATATATGAGTAAACTTGGTCTGACAGTTACCAATGCT |
| | | TAATCAGTGAGGCACCTATCTCAGCGATCTGTCTATTTCGTTCATCCATAGTTGCC |
| | | TGACTCCCCGTCGTGTAGATAACTACGATACGGGAGGGCTTACCATCTGGCCCCAG |
| | | TGCTGCAATGATACCGCGAGACCCACGCTCACCGGCTCCAGATTTATCAGCAATAA |
| | | ACCAGCCAGCCGGAAGGGCCGAGCGCAGAAGTGGTCCTGCAACTTTATCCGCCTCC |
| | | ATCCAGTCTATTAATTGTTGCCGGGAAGCTAGAGTAAGTAGTTCGCCAGTTAATAG |
| | | TTTGCGCAACGTTGTTGCCATTGCTGCAGGCATCGTGGTGTCACGCTCGTCGTTTG |
| | | GTATGGCTTCATTCAGCTCCGGTTCCCAACGATCAAGGCGAGTTACATGATCCCCC |
| | | ATGTTGTGCAAAAAAGCGGTTAGCTCCTTCGGTCCTCCGATCGTTGTCAGAAGTAA |
| | | GTTGGCCGCAGTGTTATCACTCATGGTTATGGCAGCACTGCATAATTCTCTTACTG |
| | | TCATGCCATCCGTAAGATGCTTTTCTGTGACTGGTGAGTACTCAACCAAGTCATTC |
| | | TGAGAATAGTGTATGCGGCGACCGAGTTGCTCTTGCCCGGCGTCAACACGGGATAA |
| | | TACCGCGCCACATAGCAGAACTTTAAAAGTGCTCATCATTGGAAAACGTTCTTCGG |
| | | GGCGAAAACTCTCAAGGATCTTACCGCTGTTGAGATCCAGTTCGATGTAACCCACT |
| | | CGTGCACCCAACTGATCTTCAGCATCTTTTACTTTCACCAGCGTTTCTGGGTGAGC |
| | | AAAAACAGGAAGGCAAAATGCCGCAAAAAAGGGAATAAGGGCGACACGGAAATGTT |
| | | GAATACTCATACTCTTCCTTTTTCAATATTATTGAAGCATTTATCAGGGTTATTGT |
| | | CTCATGAGCGGATACATATTTGAATGTATTTAGAAAAATAAACAAATAGGGGTTCC |
| | | GCGCACATTTCCCCGAAAAGTGCCACCTGACGTCTAAGAAACCATTATTATCATGA |
| | | CATTAACCTATAAAAATAGGCGTATCACGAGGCCCTTTCGTCTGGCCTAGGAAGCG |
| | | ACTTCCAATCGCTTTGCATATCCAGTACCACACCCACAGGCGTTTGTGCTACTCTA |
| | | CTGATAGCAATAGATGCGTCATAATTGGTTGGCCCGCTGAGCCTCCACAGGATACT |
| | | ATTGCACATACCCTGGTCATGTGCAGATCAGCTCATTTGTGGAGACTCTGGAGTAA |
| | | CTTAGACGACGCCTGGTTCAATTGCCGCAATGTGCGCCCACGCAGATAATGTATTG |
| | | AGGGGTGGAGCGCCTCTTGGGGACTTGCTGTACTTGTACGGGATATTAAACGCACT |
| | | CAGCAAGACCATGACGTAAAACACACCTACTGTACGATACGTACTGTAGGTATTGT |
| | | ACTCGTACCCGGTACTACAAATAGTACGATACTATACGGAGTGTATTTGTACCTTG |
| | | ATATACGACTGGCGGAGTGAAGAGAAGGAGTTGAACAAGACCAGATGGGGATATCA |
| | | GCCCCAGTGCTTTGTATTACAAGTACGAGTACTTAATAGATACTGTAAGGCTATTG |
| | | ATACGGATGGCAGTAAGTCATTGAGTAAGCAATTGTGGCCCAGCATCTCCCCTACG |
| | | TACTTGTACCATACCCCATGGAGACACCAATGGTCTTTCACGCACACTGTCGTGTG |
| | | CTGTATCGCAGAATCGGGTGTCCAACCAAATGCCGTTACCCCCACGTCACAGCCGA |
| | | TAGACAGATACACCATCAATACCAGCAGGTTGTATCATGCGGTTGGCTGAAGGTAA |
| | | GCTGATTGGTCTAAAAACTGTAGCTGTCCTAATTCAACGAGCGCTATTTGGGGCCA |
| | | ACCACCTCGGCCAAGCGGCCTTTAATCTGCGTGCCCCAGAGGCGTCTAATGAGGCT |
| | | CTGGCCGCCACTGTAGGAGTGTTTCTCTGTGCGCACACGCAGTTTTGAGTTTGGGC |
| | | GACTTTCCCTTTTTCCCAATTGCGTACACACACAGCTCCGAGCTAAGCGCTGTCCT |
| | | TGAACCTTCTCCCTCTTTTCCCTCTTTTTCTCTTCCCCTTCCCCTCCTCCACATTA |
| | | AGGCCAAATCCTGAATTGCACCAACTAGTACAACGACAACAATGGACAAGAAGTAC |
| | | TCCATCGGTTTGGACATTGGTACTAACTCTGTCGGCTGGGCCGTCATCACCGACGA |
| | | GTACAAGGTTCCCTCCAAGAAGTTCAAGGTCCTTGGCAACACCGACCGACACTCTA |
| | | TCAAGAAGAACCTGATCGGTGCTCTGCTGTTCGACTCTGGCGAGACTGCCGAGGCC |
| | | ACCCGACTGAAGCGAACCGCTCGACGCCGATACACCCGACGAAAGAACCGAATCTG |
| | | TTACCTCCAGGAGATCTTCAGCAACGAGATGGCTAAGGTCGACGACTCCTTCTTCC |
| | | ACCGACTCGAGGAGTCTTTCCTGGTCGAAGAGGATAAGAAGCACGAGCGACACCCC |
| | | ATCTTCGGCAACATTGTTGATGAGGTTGCCTACCATGAGAAGTACCCCACCATCTA |
| | | CCACCTCCGAAAGAAGCTCGTCGACTCCACTGACAAGGCTGACCTCCGACTCATCT |
| | | ACCTTGCTCTCGCCCACATGATCAAGTTCCGAGGTCACTTCCTCATTGAGGGTGAT |
| | | CTCAACCCCGACAACTCCGACGTTGACAAGCTGTTCATCCAGCTCGTCCAGACCTA |
| | | CAACCAGCTCTTTGAGGAGAACCCTATCAACGCTTCTGGTGTTGACGCCAAGGCCA |
| | | TTCTCTCCGCCCGACTCTCTAAGTCCCGACGACTCGAGAACCTCATTGCCCAGCTG |
| | | CCCGGCGAGAAGAAGAACGGCCTCTTCGGTAACCTGATTGCTCTCTCTCTTGGTCT |
| | | GACCCCCAACTTCAAGTCCAACTTTGACCTCGCCGAGGACGCCAAGCTCCAGCTGT |
| | | CCAAGGACACCTACGATGACGATCTGGACAACCTCCTGGCCCAGATCGGTGACCAG |
| | | TACGCCGATCTCTTCCTTGCCGCCAAGAACCTCTCCGACGCCATCCTGCTCTCCGA |
| | | CATCCTCCGAGTCAACACCGAGATTACCAAGGCTCCTCTGTCTGCCTCTATGATCA |
| | | AGCGATACGACGAGCACCACCAGGATCTCACTCTTCTCAAGGCTCTCGTCCGACAG |
| | | CAGCTCCCCGAGAAGTACAAGGAGATTTTCTTTGACCAGTCCAAGAACGGTTACGC |
| | | TGGCTACATTGACGGTGGTGCTTCCCAGGAAGAGTTTTACAAGTTCATCAAGCCTA |
| | | TTCTGGAGAAGATGGACGGTACCGAGGAGCTGCTCGTCAAGCTCAACCGAGAGGAC |
| | | CTCCTTCGAAAGCAGCGAACCTTCGATAACGGCTCCATCCCCCACCAGATCCACCT |
| | | GGGTGAGCTCCACGCCATTCTCCGAAGACAAGAGGACTTCTACCCCTTCCTAAAGG |
| | | ATAACCGAGAGAAGATCGAGAAGATTCTCACCTTCCGAATCCCCTACTACGTCGGT |
| | | CCCCTCGCTCGAGGTAACTCCCGATTTGCTTGGATGACCCGAAAGTCCGAGGAGAC |
| | | TATCACCCCCTGGAACTTTGAAGAGGTAGTCGACAAGGGTGCCTCCGCCCAGTCTT |
| | | TCATTGAGCGGATGACCAACTTCGATAAGAACCTCCCCAACGAGAAGGTCCTTCCC |
| | | AAGCACTCTCTCCTCTACGAGTACTTCACCGTCTACAACGAGCTGACCAAGGTCAA |
| | | GTACGTTACCGAGGGCATGCGAAAGCCCGCTTTCCTCTCTGGTGAGCAGAAGAAGG |
| | | CCATTGTCGACCTCCTGTTCAAGACTAACCGAAAAGTCACCGTCAAGCAGCTCAAG |
| | | GAAGACTACTTCAAGAAGATTGAGTGCTTCGACTCCGTCGAGATTTCCGGTGTCGA |
| | | GGACCGATTCAACGCCTCCCTCGGCACCTACCACGATCTTCTGAAGATCATCAAGG |
| | | ACAAGGACTTTCTTGATAACGAGGAGAACGAGGACATTCTCGAGGACATCGTCCTC |
| | | ACCCTCACCCTTTTCGAGGATCGAGAGATGATCGAGGAGCGACTCAAGACCTACGC |
| | | CCATCTCTTCGACGACAAGGTCATGAAGCAACTCAAGCGACGACGATACACTGGCT |
| | | GGGGCCGACTTTCCCGAAAGCTCATCAACGGCATCCGAGACAAGCAGTCTGGCAAG |
| | | ACCATCCTGGACTTCCTGAAGTCCGACGGTTTCGCCAACCGAAACTTCATGCAGCT |
| | | CATCCACGACGACTCTCTTACCTTCAAAGAGGATATCCAGAAGGCCCAGGTTTCTG |
| | | GCCAGGGCGACTCCCTCCACGAGCACATTGCCAACCTCGCCGGATCCCCCGCCATC |
| | | AAAAAGGGTATCCTCCAGACCGTCAAGGTTGTCGACGAACTCGTGAAGGTCATGGG |
| | | CCGACACAAGCCCGAGAACATCGTTATCGAGATGGCCCGAGAGAACCAGACCACCC |
| | | AGAAGGGTCAGAAGAACTCCCGAGAGCGAATGAAGCGAATCGAAGAGGGTATCAAG |
| | | GAGCTCGGTTCCCAGATTCTCAAGGAGCACCCCGTCGAGAACACCCAGCTCCAGAA |
| | | CGAGAAACTCTACCTGTACTACCTCCAGAATGGCCGAGACATGTACGTTGACCAGG |
| | | AGCTCGACATCAACCGACTCTCCGACTACGACGTCGACCACATTGTTCCTCAGTCC |
| | | TTCCTCAAGGACGACTCCATCGACAACAAGGTTCTGACCCGATCTGACAAGAACCG |
| | | AGGTAAGTCCGACAACGTTCCCTCCGAAGAGGTCGTTAAGAAGATGAAGAACTACT |
| | | GGCGACAGCTTCTCAACGCCAAACTGATCACCCAGCGAAAGTTTGACAACCTCACC |
| | | AAGGCCGAGCGAGGTGGTCTGTCCGAGCTGGACAAGGCCGGCTTCATTAAGCGACA |
| | | GCTGGTCGAGACTCGACAGATCACCAAGCACGTCGCCCAGATCCTCGACTCCCGAA |
| | | TGAACACCAAGTACGACGAGAACGACAAGCTCATCCGGGAGGTCAAGGTCATCACC |
| | | CTGAAGTCTAAGCTTGTCTCCGACTTCCGAAAGGACTTCCAGTTCTACAAGGTCCG |
| | | AGAGATCAACAACTACCACCACGCCCACGACGCCTACCTCAACGCCGTTGTTGGTA |
| | | CCGCCCTCATCAAGAAGTATCCCAAGCTCGAGTCCGAGTTCGTTTACGGCGACTAC |
| | | AAGGTTTACGATGTCCGAAAGATGATTGCCAAGTCCGAGCAGGAGATCGGTAAGGC |
| | | CACCGCCAAGTACTTTTTCTACTCCAACATCATGAATTTCTTCAAGACCGAGATCA |
| | | CTCTCGCCAACGGTGAGATTCGAAAGCGACCCCTGATTGAGACTAATGGTGAGACT |
| | | GGTGAGATCGTCTGGGATAAGGGCCGAGACTTCGCCACCGTCCGAAAGGTCCTGTC |
| | | CATGCCCCAGGTCAACATTGTCAAGAAGACCGAGGTCCAGACCGGTGGCTTCTCCA |
| | | AGGAGTCCATTCTCCCCAAGCGAAACTCCGACAAACTCATCGCCCGTAAGAAGGAC |
| | | TGGGATCCGAAGAAGTACGGTGGTTTCGATTCTCCCACCGTTGCCTACTCCGTCCT |
| | | CGTTGTTGCTAAAGTCGAGAAGGGTAAGTCTAAGAAACTCAAGTCCGTGAAGGAGC |
| | | TACTCGGTATCACCATCATGGAGCGATCTTCTTTTGAGAAGAACCCCATTGACTTC |
| | | CTCGAGGCCAAGGGTTACAAAGAGGTCAAGAAGGACCTGATTATCAAGCTGCCCAA |
| | | GTACTCCCTCTTTGAGCTCGAGAACGGCCGAAAGCGAATGCTGGCTTCCGCTGGTG |
| | | AGCTGCAGAAGGGCAACGAGCTCGCTCTGCCCTCCAAGTACGTCAACTTCCTCTAC |
| | | CTGGCCTCCCACTACGAGAAGCTCAAGGGCTCCCCCGAGGACAACGAGCAGAAGCA |
| | | GCTGTTCGTTGAGCAGCACAAGCACTACCTCGACGAGATCATCGAGCAGATCTCCG |
| | | AGTTCTCCAAGCGAGTCATCCTCGCTGACGCCAACCTTGATAAGGTTCTCTCTGCT |
| | | TACAACAAGCACCGGGACAAGCCCATCCGAGAGCAGGCCGAGAATATCATCCACCT |
| | | CTTCACTCTCACCAACCTCGGCGCTCCTGCTGCCTTCAAGTACTTCGACACCACCA |
| | | TTGACCGAAAGAGGTACACCTCCACCAAGGAAGTCCTCGACGCCACCCTGATCCAC |
| | | CAGTCCATCACCGGCCTCTACGAAACCCGAATCGACCTCTCCCAGCTCGGCGGTGA |
| | | CTCTCGAGCCGACCCCAAGAAGAAGCGAAAAGTCTAAATATCCGAAGATCAAGAGC |
| | | GAAGCAAGTTGTAAGTCCAGGACATGTTTCCCGCCCACGCGAGTGATTTATAACAC |
| | | CTCTCTTTTTTGACACCCGCTCGCCTTGAAATTCATGTCACATAAATTATAGTCAA |
| | | CGACGTTTGAATAACTTGTCTTGTAGTTCGATGATGATCATATGATTACATTAATA |
| | | GTAATTACTGTATTTGATATATATACTAATTACAATAGTACATATTAGAACATACA |
| | | ATAGTTAGTGCCGTGAAGTGGCTTAAAATACCGCGAGTCGATTACGTAATATTATT |
| | | ACCTCTTGCCCATCGAACGTACAAGTACTCCTCTGTTCTCTCCTTCCTTTGCTTTG |
| | | TGCACGAAGAACTGCGGTCAGGTGACACAACTTTTTCCATCTCAGGGTGTGTCGCG |
| | | TGTGCTTCATCCAAACTTTAGTTGGGGTTCGGGTTCGCGCGAGATGATCACGTGCC |
| | | CTGATTTGGTGTCGTCCCCCGTCGCGCTGCGCACGTGATTTATTTATTTCCGGTGG |
| | | CTGCTGTCTACGCGGGGCCTTCTCTGCCCTTCTGTTTCAACCTTCGGGCGGTTCTC |
| | | GTAACCAGCAGTAGCAATCCATTTCGAAACTCAAAGAGCTAAAAACGTTAAACCTC |
| | | AGCAGTCGCTCGACGAATGGGCTGCGGTTGGGAAGCCCACGAGGCCTATAGCCAGA |
| | | GCCTCGAGTTGACAGGAGCCCAGACGCCTTTTCCAACGGCAACTTTTATATAAAAT |
| | | GGCAATGTATTCATGCAATTGCGGCCGTGTCAGGTTGGAGACACTGGACCACACTC |
| | | TCCATTGCTTCCTGAGGAGATGGATCATTGCTAGTGCATCTACGCGCAGCAATCCC |
| | | GCAAGCTCGACAACCGTAGATGGGCTTTGGTGGGCCAATCAATTACGCAACCCGCA |
| | | CGTTAAATTGTATGAGGAAGGAAGGCCACGGTACAAAGTGGGTGGTCTTCACCCAG |
| | | TGGTTGTTGGTGGCGTCATGCAGACCATGCATTGGGGATAGCACAGGGTTGGGGTG |
| | | TCTTGTGGACTCAATGGGTGAAAGGAGATGGAAAAGGGCGGTGAAAAGTGGTAGAA |
| | | TCGAAATCCCTGACGTCAATTTATAAAGTAAAATGCGTTTCTGCCATTTTGCTCCC |
| | | CTCCTTCTTTCGCAATCGCCTCCCCAAAAGTTGTCGTGGCAGTACACATGCTTGCA |
| | | TACAATGAAGCTAATCCGGCTTGCTCAGTAGTTGCTATATCCAGGCATGGTGTGAA |
| | | ACCCCTCAAAGTATATATAGGAGCGGTGAGCCCCAGTCTGGGGTCTTTTCTCTCCA |
| | | TCTCAAAACTACTTTCTCACAATGCTGCATCTGATGAGTCCGTGAGGACGAAACGA |
| | | GTAAGCTCGTCATGCAGCTCTTCAACGACGCGTTTTAGAGCTAGAAATAGCAAGTT |
| | | AAAATAAGGCTAGTCCGTTATCAACTTGAAAAAGTGGCACCGAGTCGGTGCTTTTG |
| | | GCCGGCATGGTCCCAGCCTCCTCGCTGGCGCCGGCTGGGCAACATGCTTCGGCATG |
| | | GCGAATGGGACTAAACTTCGAGCTAATCCAGTAGCTTACGTTACCCAGGGGCAGGT |
| | | CAACTGGCTAGCCACGAGTCTGTCCCAGGTCGCAATTTAGTGTAATAAACAATATA |
| | | TATATTGAGTCTAAAGGGAATTGTAGCTATTGTGATTGTGTGATTTTCGTCTTGCT |
| | | GGTTCTTATTGTGTCCCATTCGTTTCATCCTGATGAGGACCCCTGGAACCGGTGTT |
| | | TTCTTAGTCTCTGCAATCGCTAGTCTTGTTGCTATGACAGTTGCGTCGACACTATT |
| | | CAGGTCATCTATCGGTTATTCTGATATTATAATACCTCCGGATCGATGTACCTGAT |
| | | TTATACTTGCAGCAATGTTTACTTCTTATCGCGATACACGAATGTGATACGGATCA |
| | | AAGTAAGCAGGACTACGATAAGATAACGAATGCGGTGCAGTCCATGTCGATTAGGT |
| | | ATAGATACATTTATTTTGTGTTATGTTACATTTTGGGGGGATACTGTCCTACTTGT |
| | | AGTACCTACTTGTAGTGGCGCGTCTATTCCTTTGCCCTCGGACGAGTGCTGGGGCG |
| | | TCGGTTTCCACTATCGGCGAGTACTTCTACACAGCCATCGGTCCAGACGGCCGCGC |
| | | TTCTGCGGGCGATTTGTGTACGCCCGACAGTCCCGGCTCCGGATCGGACGATTGCG |
| | | TCGCATCGACCCTGCGCCCAAGCTGCATCATCGAAATTGCCGTCAACCAAGCTCTG |
| | | ATAGAGTTGGTCAAGACCAATGCGGAGCATATACGCCCGGAGCCGCGGCGATCCTG |
| | | CAAGCTCCGGATGCCTCCGCTCGAAGTAGCGCGTCTGCTGCTCCATACAAGCCAAC |
| | | CACGGCCTCCAGAAGAAGATGTTGGCGACCTCGTATTGGGAATCCCCGAACATCGC |
| | | CTCGCTCCAGTCAATGACCGCTGTTATGCGGCCATTGTCCGTCAGGACATTGTTGG |
| | | AGCCGAAATCCGCGTGCACGAGGTGCCGGACTTCGGGGCAGTCCTCGGCCCAAAGC |
| | | ATCAGCTCATCGAGAGCCTGCGCGACGGACGCACTGACGGTGTCGTCCATCACAGT |
| | | TTGCCAGTGATACACATGGGGATCAGCAATCGCGCATATGAAATCACGCCATGTAG |
| | | TGTATTGACCGATTCCTTGCGGTCCGAATGGGCCGAACCCGCTCGTCTGGCTAAGA |
| | | TCGGCCGCAGCGATCGCATCCATGGCCTCCGCGACCGGCTGCAGAACAGCGGGCAG |
| | | TTCGGTTTCAGGCAGGTCTTGCAACGTGACACCCTGTGCACGGCGGGAGATGCAAT |
| | | AGGTCAGGCTCTCGCTGAATTCCCCAATGTCAAGCACTTCCGGAATCGGGAGCGCG |
| | | GCCGATGCAAAGTGCCGATAAACATAACGATCTTTGTAGAAACCATCGGCGCAGCT |
| | | ATTTACCCGCAGGACATATCCACGCCCTCCTACATCGAAGCTGAAAGCACGAGATT |
| | | CTTCGCCCTCCGAGAGCTGCATCAGGTCGGAGACGCTGTCGAACTTTTCGATCAGA |
| | | AACTTCTCGACAGACGTCGCGGTGAGTTCAGGCTTTTTCATATGGGTACCTGAGAA |
| | | CATTTTTGTGTCTAGGTGTTTGTGTTTGGACTGCGATCAGTGAAGAAAAGAAGAGG |
| | | AAAAATTGTGCAAGAAATTTTGCTTTCAAGACTTGGCTGATGCAGCAGGGTAACTC |
| | | TGGGACACAGACCTATGTTTGTGGTTAAACTCAATGCACGTGGTACGTGCGTGGAG |
| | | CGCTTACCCATCCAAGGGTGTGGACATGGAACCGACGGTCCGTGGAGTTGTGTAAT |
| | | GTCATTTTGGCGACTCTTGAAGCAAGGCTATAAAAAAATTGTGTGGCTTGAGTCTT |
| | | ATCGAGCTCGGTCACTACAAGAGTTAATCTTCCTGTCTCAGGCAGACAGGTCAGGC |
| | | AGGGTTACTTTTGGGTGTGCTGTAACTCACTGTATGGCCGTTAGTGCGCATAGACG |
| | | TTGTACATACTGGACCGAATTGTAGCGTGCTCAATAGGGCCAATAAAGCTATTGTA |
| | | GGGATCCGAATTTTCAGAACCTAATTTATCTGTTACCCGGCCTGTGGCTCGCACAG |
| | | CTTAAAAATGGTCAAACTTTCCCCTTCTTGTCTTTTTTTCCTCACATTCATCAGGT |
| | | TCTTGTCTTGATCTTTCAAGTGAGTATTAATTACCGACCTTGGTTCTTCATTGGGA |
| | | GAGCATTGGAAGCCGTGGTGCAGCAACCACAAAACGGTTCTTCCCCTTCGATACCT |
| | | TCTTGCCTGCCTTTCAATACAAGTCGGCTCGATTAGCGGTGGTCGCCCCCGCCAGC |
| | | GGAGAACATGGAACTAACCCAGAATGAGAGCTAAGTGGAGAAAGAAGAGAGTCAGA |
| | | CGACTCAAGCGAAAGCGCCGCAAGGTCCGAGCTCGATCCAAATAAGCGGTTTTTAA |
| | | CGGAGATTTAACACTAAATCGAAGAACTTTTCCCGTTTCATTTGCGAATGAGCTCG |
| | | TTAACAAAATCCCCCAGTTTTTTTATCCAGCTGTAAGGATTGACATTAGTAATGAA |
| | | TTATTGTTTGGTATATTTAAATCTGTAGTTCCTTTCTGTCCGTGTCGGCAACTGTC |
| | | GTACTCGTGATTTACTTGTATTGACGAATACTTACTGTAGCGCACTCTGCTGCTAC |
| | | TGGTCGTAAGGATGTGCTATTTCGGTGTATGGTGGGTTTTTTGGGGGTCGGAACCG |
| | | AAGACTGTTACACGGGCACGGCTCGTTGTGTACACGCACAGAGCTCTTGCGAGTCA |
| | | TGTTGTAGCTAGCTCGTCGTGTTCAGGAACTGTTCGATGGTTCGGAGAGAGTCGCC |
| | | GCCCAGAACATACGCGCACCGATGTCAGCAGACAGCCTTATTACAAGTATATTCAA |
| | | GCAAGTATATCCGTAGGGTGCGGGTGATTTGGATCTAAGGTTCGTACTCAACACTC |
| | | ACGAGCAGCTTGCCTATGTTACATCCTTTTATCAGACATAACATAATTGGAGTTTA |
| | | CTTACACACGGGGTGTACCTGTATGAGCACCACCTACAATTGTAGCACTGGTACTT |
| | | GTACAAAGAATTTATTCGTACGAATCACAGGGACGGCCGCCCTCACCGAACCAGCG |
| | | AATACCTCAGCGGTCCCCTGCAGTGACTCAACAAAGCGATATGAACATCTTGCGAT |
| | | GGTATCCTGCTGATAGTTTTTACTGTACAAACACCTGTGTAGCTCCTTCTAGCATT |
| | | TTTAAGTTATTCACACCTCAAGGGGAGGGATAAATTAAATAAATTCCAAAAGCGAA |
| | | GATCGAGAAACTAAATTAAAATTCCAAAAACGAAGTTGGAACACAACCCCCCGAAA |
| | | AAAAACAACAAACAAAAAACCCAACAAAATAAACAAAAACAAAATAAATATATAAC |
| | | TACCAGTATCTGACTAAAAGTTCAAATACTCGTACTTACAACAAATAGAAATGAGC |
| | | CGGCCAAAATTCTGCAGAAAAAAATTTCAAACAAGTACTGGTATAATTAAATTAAA |
| | | AAACACATCAAAGTATCATAACGTTAGTTATTTTATTTTATTTAATAAAAGAAAAC |
| | | AACAAGATGGGCTCAAAACTTTCAACTTATACGATACATACCAAATAACAATTTAG |
| | | TATTTATCTAAGTGCTTTTCGTAGATAATGGAATACAAATGGATATCCAGAGTATA |
| | | CACATGGATAGTATACACTGACACGACAATTCTGTATCTCTTTATGTTAACTACTG |
| | | TGAGGCATTAAATAGAGCTTGATATATAAAATGTTACATTTCACAGTCTGAACTTT |
| | | TGCAGATTACCTAATTTGGTAAGATATTAATTATGAACTGAAAGTTGATGGCATCC |
| | | CTAAATTTGATGAAAGATGAAATTGTAAATGAGGTGGTAAAAGAGCTACAGTCGTT |
| | | TTGTTTTGAGATACCATCATCTCTAACGAAATATCTATTAAAAATCTCAGTGTGAT |
| | | CATGAGTCATTGCCATCCTGGAAAATGTCATCATGGCTGATATTTCTAACTGTTTA |
| | | CTTGAGATAAATATATATTTACAAGAACTTCCCTTGAAATTAATTTAGATATAAAA |
| | | TGTTTGCGGGCAAGTTACTACGAGGAATAAATTATATCTAGA |

TABLE 2
list of Yarrowia lipolytica strains used. For more details, see text.

| Strain | Description | Reference |
|---|---|---|
| ML13961 | MATB prototrophic wild type | This patent |
| ML13701 | ML13961 with mnn9_del | This patent |
| ML12819 | AXN-accumulating Yarrowia lipolytica | WO2014096992 |
| ML17008 | ML12819 with mnn9_del | This Patent |
| ML17012 | ML12819 with chs4_del | This Patent |
| ML17226 | ML12819 with mnn9_chs4_del | This Patent |
| ML17228 | ML12819 with mnn9_chs4_del | This Patent |
| ML17010 | ML12819 with gas1_del | This Patent |

Fermentation conditions. As performed in WO2014096992.

### Example 2: Production of AXN in an MNN9 Deletion Strain

Strains of Yarrowia lipolytica w/o deletion of endogenous mnn9 gene where constructed and cultivated as described (see Ex. 1). Biomass was generated by the strains of interest in shake flasks. In preparation for the inoculation of the shake flasks, YPD plates were inoculated from glycerol stocks of each strain. The plates were grown at 26° C. for 48 hours. Biomass from plates for each strain was used to inoculate twelve baffled 250 ml shake flasks. The shake flasks had been prepared with 50 ml YPO media (1% yeast extract, 2% peptone, 5% soy oil), to yield 500 ml of culture. Each plate was scraped with a 10 μl sterile inoculating loop to recover biomass that was approximately 0.125 cm³. The biomass was placed in sterile tubes that had been prepared with 1 ml sterile PBS. The biomass was dispersed in the PBS before inoculating each flask with 100 μl of the suspension. The flasks were placed in a 26° C. incubator and agitated at 250 rpm for 90 hours. The total dry matter content at harvest for each shake flask sample was measured, being in the range of 5.9 to 6.3 wt % for both ML13961 and ML12819 based strains, either with or without deletion of mnn9.

The cell wall integrity or lysis phenotype of the Yarrowia strains was tested either by enzymatic lysis and/or by bead milling.

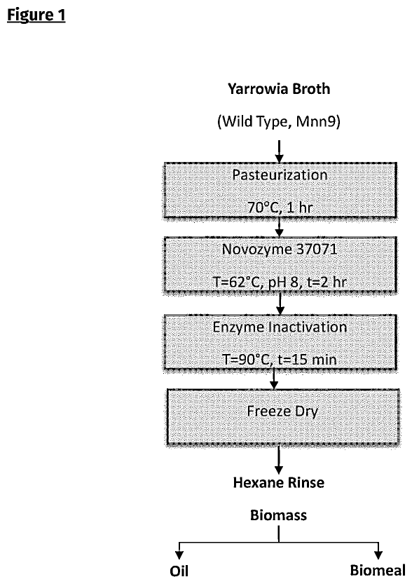

For enzyme pre-treatment experiments, Yarrowia Broth (ML13961 and ML12819-based strains) was harvested from shake flasks pooled to generate a composite sample for downstream processing (see FIG. 1). The pooled broth samples were pasteurized on the bench (T=70° C., t=1 hr) using 3-neck flasks equipped with overhead stirring and heating mantles. Following pasteurization, each sample was treated with Alcalase® (Novozymes; 1 wt %/total dry matter, T=62° C., pH 8, t=2 hr). Following the 2-hour treatment time, Alcalase® was inactivated by increasing the temperature to 90° C. and holding for 15 minutes. Samples were withdrawn after pasteurization and enzyme pre-treatment steps and were analyzed under the microscope to determine if cell disruption had occurred after each process step. Besides differences in cell morphology, the appearance of “ghost” cells was noted in the AXN-accumulating ML17008 sample carrying the mnn9 deletion after enzyme treatment, suggesting the potential release of intracellular carotenoid.

Each enzyme pre-treated biomass sample was transferred to a Buchner funnel equipped with Whatman 1 filter paper. The biomass was rinsed with hexane (1:4 w/w, biomass:hexane) under vacuum, applying hexane in 2 mL aliquots until the measured amount of solvent was used. The flowthrough was collected, and the hexane was evaporated using a rotary evaporator, concentrating the free oil phase. Mass balance and solids balance closures were calculated: mass balances closed within 99-100% and solids balances closed between 101-104%.

When analyzing the hexane rinse fractions it was noted that the extract of the strain ML17008 was an order of magnitude darker in appearance when compared to the ML12819 strain, indicating that at least 5-fold more carotenoid was extracted in the mnn9 knockout Astaxanthin strain. Thus, wherein the color of hexane rinse fraction of ML12819 corresponds to Pantone 103, 109, 110, 111, 117, 129, 397, 398, 458, 605, 606, 612, the color of hexane rinse fraction of ML17008 corresponds more to Pantone 137, 138, 144, 145, 146, 151, 152, 153, 158, 471, 717, 1505, 1575 (Pantone Matching System©).

Crude oil extract, hexane washed biomeal, and enzyme treated biomass were submitted for FAME analysis. This data was utilized to calculate the lipid distribution in the oil phase and rinsed biomeal solids (Table 3).

TABLE 3
distribution of recovered AXN (lipids) from pre-treated
fermentation broths and extraction with hexane measured
by FAME analysis. Numbers are normalized to 100%.
For more details see text or FIG. 1.

| | MNN9 | Normalized | Normalized oil loss |
|---|---|---|---|
| Strain | genotype | oil yield | in biomeal |
| ML12819 | wt | 1% | 99% |
| ML17008 | Δmnn9 | 8% | 92% |
| ML 13961 | wt | 5% | 95% |
| ML13701 | Δmnn9 | 7% | 93% |

Comparing the oil yield, it was observed that the effect of the deletion in the endogenous MNN9 gene is more prominent in an AXN-overproducing background, i.e. strains based on ML12819, compared to the effect in a non-AXN-overproducing strain background, with the highest free oil recovery (8%) of lipid recovered as oil and 92% lipid remaining in the biomass. Thus, the distribution of lipid as oil increased by 40% in a non-AXN producing strain background as compared to 700% (from 1% to 8% via introduction of MNN9-deletion) in AXN-producing strain background.

This finding may suggest that there is a preferential extraction of lipids in the presence of carotenoid.

For bead-milling experiments, strains were grown in a 5 L fermentor and 1 L of the broth was milled by maximum of 4 passes (see Ex.1). ML13961-based strains showed no enhanced cell rupture as tested by hemocytometer or viscosity, indicating that the MNN9-deletion had no effect on physical lysis by bead mill. However, testing AXN-producing ML12819-based strains by direct cell count and visual assessment of viscosity showed enhanced lysis after each of the first 2 or 3 passes (Table 4).

TABLE 4
effect of bead milling in an AXN-overproducing strain w/o
deletion of endogenous MNN9. For more details see text.

| | |
|---|---|
| Cell Count | % Cell Rupture |

| Pass | ML12819 | ML17008 | ML12819 | ML17008 |
|---|---|---|---|---|
| 0 | 102 | 140 | 0 | 0 |
| 1 | 63 | 56 | 39 | 60 |
| 2 | 50 | 35 | 51 | 75 |
| 3 | 19 | 27 | 81 | 81 |
| 4 | 17 | 15 | 84 | 89 |

Further to the effect of MNN9 deletion with regards to cell rupture in an AXN-producing Yarrowia strain, the effect of other cell wall integrity genes was evaluated. Strains were constructed as described in Ex.1 for the MNN9 deletion. Strain ML17012 (AXN-producing strain with deletion in CHS4) showed cell rupture of 70% after 2 passes, 80% after 4 passes. The other strains tested did not show any significant effect in bead-milling experiments.

Using a strain wherein both endogenous MNN9 and CHS4 are deleted (ML17228, see Tab. 2), the cell rupture could be increased to 80% after 2 passes, with a maximum of 87% after 4 passes.

To evaluate whether the MNN9 deletion would have an impact on extraction of AXN, particularly acetylated-AXN, fermentation and bead milling experiments were repeated and the resulting samples tested for % extractability (acetyl-AXN acetone extracted/total intracellular acetyl-AXN). Thus, cells were grown in 5 L fermentors and 1.5 L of broth was milled up to four passes through the bead mill with samples taken prior to any milling and after each pass (see Ex. 1). Similar to previous experiments the mnn9 deletion strain (AXN-producing strain) showed no deleterious impact on product titer but did show enhanced lysis after each of the first 2 passes (Table 5). Evaluation of acetyl-AXN yield per bead mill pass using AXN-strains comprising cell wall integrity mutations (deletion of endogenous gas1, chs4) and a double mutant mnn9_chs4_del (strain ML17228) is shown in Table 6.

TABLE 5
effect of bead milling in an AXN-overproducing strain
w/o deletion of endogenous MNN9 with regards to extraction
of acetylated AXN. For more details see text.

| | % Acetyl-AXN extracted | | | | |
|---|---|---|---|---|---|
| | Bead Mill Passes | | | | |
| Strain | 0 | 1 | 2 | 3 | 4 |
| ML12819 | 34 | 63 | 77 | 82 | 84 |
| ML17008 | 54 | 80 | 85 | 83 | 83 |

TABLE 6
effect of bead milling in an AXN-overproducing strain w/o
deletion of endogenous gas1, chs4, mnn9_chs4 with regards
to extraction of acetylated AXN. For more details see text.

| | % Acetyl-AXN extracted | | | | |
|---|---|---|---|---|---|
| | Bead Mill Passes | | | | |
| Strain | 0 | 1 | 2 | 3 | 4 |
| ML12819 | 38 | 66 | 77 | 79 | 81 |
| ML17010 | 54 | 67 | 76 | 80 | 81 |
| ML17012 | 68 | 81 | 84 | 85 | 82 |
| ML17226 | 64 | 79 | 82 | 81 | 79 |

Strains ML17012 (chs4_del) showed higher % extraction before bead milling and after passes 1-3. Strain ML17010 (gas1_del) showed higher % extraction than ML12819 before bead milling, but not after any subsequent bead mill passes.

### Example 3: Effect of Enzyme Pre-Treatment on Mutant AXN-Production Strains

Strains ML17008 and ML17012 were further pre-treated with different enzymes to check the effect on acetyl-AXN extraction. Thus, strains were pre-treated with a broad specificity protease (Alcalase®, Novozymes) and a R-glucanase, cellulase and xylanase preparation (Filtrase® NL, DSM) according to Ex. 1, followed by acetone extraction as described before. The effect on cell rupture is shown in Table 7.

TABLE 7
effect of enzyme pre-treatment by Alcalase ® (“ALC”) and
a mix of Alcalase ®/Filtrase ®NL (“ALC/BGF”) on
cell rupture of gas1 or chs4_del strain. For more details see text.

Cell rupture [%]

| Strain | ALC | ALC/BGF |
|---|---|---|
| ML12819 | 52 | 75 |
| ML17010 | 29 | 62 |
| ML17013 | 68 | 95 |

Pre-treatment of said enzymes had also an effect on AXN extraction. Even before bead-milling and after pass 1 & 2, strain ML17008 showed higher % extraction (of total intracellular AXN). Use of ML17012 together with mixed ALC/BGF resulted in increased extraction yield of acetyl-AXN, from 27 to 79%. The results are shown in Table 8.

TABLE 8
effect of enzyme pre-treatment on AXN extraction
using enzyme mix of Alcalase ®/Filtrase ®NL (“ALC/BGF”).
For more details see text.

AXN extraction [%]

| Strain | control | ALC/BGF |
|---|---|---|
| ML12819 | 26 | 61 |
| ML17010 | 20 | 49 |
| ML17013 | 27 | 79 |

A comparison of the different strains for their % of AXN extractability without any bead milling after extraction with acetone is shown in Table 9.

TABLE 9
% AXN extractability calculated on total AXN =
100% (“% AXN”) for the different strains with
acetone extraction and no milling. For more details see text.

| Strain | % AXN |
|---|---|
| ML12819 | 34-40 |
| ML17008 | 65-70 |
| ML17012 | 67 |
| ML17010 | 54 |
| ML17228 | 65 |

Thus, acetone extraction of AXN could be increased by about at least 2× with introduction of MNN9 deletion into the host cell.

1. A genetically modified host cell, particularly oleaginous yeast, accumulating intracellular lipophilic substances such as fat-soluble vitamins, carotenoids or polyunsaturated fatty acids (PUFAs), particularly carotenoids, said host cell comprising a mutation in an endogenous gene involved in the N-glycosylation pathway.

2. The modified host cell of claim 1, wherein the activity of the endogenous gene encoding for mannan polymerase complexes subunit MNN9 is reduced or abolished.

3. The modified host cell according to claim 1, furthermore comprising reduced or abolished activity of endogenous genes involved in cell wall integrity.

4. The modified host cell according to claim 1, wherein the oleaginous yeast is Yarrowia, particularly Yarrowia lipolytica.

5. The modified host cell according to claim 1, wherein the MNN9 is selected from a polypeptide with at least about 50%, such as 60, 70, 80, 90, 95, 98, or 100% identity to SEQ ID NO:1.

6. The modified host cell according to claim 1, wherein the mannan polymerase complexes subunit MNN9 is a null mutant, more preferably wherein the mannan polymerase complexes subunit MNN9 is disrupted.

7. The modified host cell according to claim 1, wherein the carotenoid accumulated in the cell is selected from astaxanthin, zeaxanthin, canthaxanthin, β-cryptoxanthin, lutein, lycopene, rhodoxanthin, beta-carotene, alpha-carotene or gamma-carotene.

8. The modified host cell according to claim 1, wherein the accumulation of carotenoid is in the range of at least 1 g/l.

9. Use of the modified host cell according to claim 1 for the production of carotenoids, wherein the percentage of carotenoids is increased by at least about 2 to 10%.

10. A process for production of carotenoids in an oleaginous host cell, comprising:
(a) providing a modified host cell according to claim 1,
(b) cultivating said host cell under conditions that the carotenoids are intracellularly accumulated, and
(c) extraction of the carotenoid from the cells.

11. The process according to claim 10, wherein the carotenoid is selected from astaxanthin, zeaxanthin, canthaxanthin, β-cryptoxanthin, lutein, lycopene, rhodoxanthin, beta-carotene, alpha-carotene or gamma-carotene.

12. The process according to claim 10, wherein the carotenoids are extracted with a composition comprising dichloromethane, acetone, ethyl acetate, isobutyl acetate, octanol, and/or hexane, preferably a non-dichloromethane-comprising composition.

13. The process according to claim 10, wherein before extraction of the carotenoid the cells are submitted to enzymatic lysis.

14. The process according to claim 13, wherein enzymatic lysis comprises addition of proteases, zymolases, chitinases, mannanases, glucanases, alcalases, xylanases, cellulases and/or mixtures thereof, preferably compositions comprising alcalases, xylanases, glucanases and/or cellulases.
